(19) United States
(12) Patent Application Publication
(10) Pub. No.: US 2016/0341712 A1
(43) Pub. Date: Nov. 24, 2016

(54) SYSTEM AND METHOD FOR ANALYZING TISSUE INTRA-OPERATIVELY USING MASS SPECTROMETRY

(71) Applicant: BRIGHAM AND WOMEN'S HOSPITAL, INC., Boston, MA (US)

(72) Inventor: Nathalie AGAR, Newton, MA (US)

(21) Appl. No.: 15/031,375

(22) PCT Filed: Oct. 23, 2014
(86) PCT No.: PCT/US2014/062017

§ 371 Date: Apr. 22, 2016

Related U.S. Application Data
(60) Provisional application No. 61/894,595, filed on Oct. 23, 2013.

(51) Int. Cl. G01N 33/483; G01N 33/574; A61B 90/00; A61B 34/20; A61B 10/02; A61B 17/32

(52) U.S. Cl. CPC G01N 33/4833; A61B 10/02; A61B 17/320068; A61B 90/36; A61B 34/20; A61B 90/37; G01N 33/57496; A61B 2218/007; A61B 2034/2051; A61B 2090/374; A61B 2090/3762; G01N 2800/52

(57) ABSTRACT

A system and method for ample analysis including acquiring a tissue sample, preparing the tissue sample for mass spectrometry imaging, conducting a mass spectrometry procedure on the tissue sample to produce an image, analyzing the image to determine the presence or absence of a biomarker; and generating a report indicating a presence or absence of cancer.

### CROSS-REFERENCE TO RELATED APPLICATIONS

This application claims priority to U.S. Provisional Patent Application No. 61/894,595, filed Oct. 23, 2013, the entire contents of which are incorporated herein by reference.

### BACKGROUND

The invention relates generally to intra-operative diagnostics of sample tissues. More specifically, the invention relates to the use of mass spectrometry for the detection of specific biomarkers.

Cancer presents many highly complex issues in clinical medicine. For example, consider just one of the many different and varied types of cancer, such as breast cancer. As a severely malignant and invasive tumor, breast cancer is a leading cause of death in cancerous women. Surgical removal of a cancerous tumor is usually the initial treatment of breast cancer, either by lumpectomy or mastectomy. Most women have a preference for the less invasive lumpectomy, for example, because of the cosmetic appearance. However, the accurate intra-operative determination of a tumor margin is challenging when planning and performing a breast-conserving surgery.

Normally, breast surgeons remove the tumor along with a few centimeters of surrounding healthy tissues on the basis of preoperative imaging using mammography, ultrasonography, or magnetic resonance imaging (MRI) to ensure the complete resection of cancer. Although accurate tumor size assessment may be available, the lack of real-time imaging in conjunction with surgical procedures relative to these techniques affects the surgery success rate and oftentimes leads to the need for further operations, giving the risk of local recurrence of breast cancer after lumpectomy and leading to a higher incidence of mastectomy. Therefore, the development of a technique allowing fast and in situ diagnosis and accurate characterization of a tumor margin boundary would facilitate a breast surgeon's decision making during lumpectomy.

The intra-operative application of MRI has been newly developed, especially in brain surgery. However, instead of providing real time imaging, this technique still requires the surgery to be interrupted. Ultrasonography has been applied intra-operatively in breast cancer excision, but it is unreliable in detecting nonpalpable tumor or ductal carcinoma in situ lesions. Positron emission tomography (PET), and near-infrared fluorescence (NIRF) optical imaging are two techniques that are being developed for intra-operative tumor assessment. However, the incorporation of radioactive or fluorescent labels presents a disadvantage not only to the patient but also to the operative personnel repeated exposure.

The review of tissue sections by light microscopy remains a cornerstone of tumor diagnostics. In recent decades, monitoring expression of individual proteins using immunohistochemistry and characterizing chromosomal aberrations, point mutations and gene expression with genetic tools has further enhanced diagnostic capabilities. These ancillary tests, however, often require days to perform and results become available long after surgery is completed. For this reason, the microscopic review of tissue biopsies frequently remains the sole source of intraoperative diagnostic information, with many important surgical decisions based on this information. This approach is time consuming, requiring nearly 30 minutes between the moment a tissue is biopsied and the time the pathologist's interpretation is communicated back to the surgeon. Tools that provide immediate feedback to the surgeon could transform the way surgery is performed.

Stereotactic surgical procedures were developed in the early 1900's and were first applied clinically in the 1940's (Kelly, P., Neurosurgery 46:16 (2000)). Initially these procedures were used in neurosurgery and involved affixing an external apparatus to a patient's skull to establish a coordinate system for locating, in a reproducible manner, the exact position of a lesion within the intracranial area. Today, stereotactic procedures have been applied to other tissues and are typically used in conjunction with diagnostic imaging procedures such as CT scans and MRIs to map internal tissues, prior to, or during, surgery (see, e.g., Poza, et al., Appl. Neurophysiol., 48:482-487 (1985); Dorwald, et al. Br. J. Neurosurg. 16:110-118 (2002); Krieger, et al., J. Surg. Oncol. 14:13-25 (1998)).

The development of stereotactic methods and imaging techniques has been accompanied by the development of surgical instruments that allow physicians to perform procedures at sites that were formerly inaccessible. Among the most successful of the instruments that have been developed for neurosurgery are probes designed to ultrasonically ablate tissue. For example, the Cavitron Ultrasonic Surgical Aspirator® (Integra Radionics) uses pulses of ultrasonic energy delivered to a needle-like tip to fragment tissue, which is concurrently irrigated and removed by aspiration. Although probes of this type were initially designed primarily for the surgical resection of tumors, it was subsequently found that the tissue fragments generated by the devices maintain sufficient integrity to be used diagnostically (Richmond, et al., Neurosurg. 13:415-419 (1983); Malhotra, et al., Acta Neurochir. 81:132-134 (1986); Blackie, et al., J. Clin. Pathol. 37:1101-1104 (2008)).

In addition to probes that ablate tissue ultrasonically, probes such as the Nico Myriad™ probe (NICO Corporation) have been designed to perform surgical ablations by mechanically cutting or shaving tissue. One attractive aspect of these “mechanical sampling” probes is that tissue is obtained without the generation of heat.

Despite the advances noted above, the diagnostic use of ultrasonic and mechanical probes has gone largely undeveloped and potential advantages over traditional methods of tissue sampling have often gone unrecognized.

Thus, a need exists for an intra-operative diagnostic solution that provides a surgeon with more information about a tumor margin boundary.

### BRIEF SUMMARY OF THE INVENTION

The present invention overcomes the aforementioned drawbacks by providing a system for utilizing mass spectrometry within the procedure room to provide real time feedback concerning the presence of cancerous cells at the surgery boundary. A hand held sampling probe can be used that allows a surgeon to collect samples intra-operatively from target areas of a surgery site. One exemplary probe is disclosed in U.S. Patent Publication No. 2011/0144476, the entirety of which is incorporated herein by reference.

One aspect of the present invention provides a system for determining a presence of cancer in a tissue sample. The system includes a sampling probe including a tip configured to vibrate in response to ultra-sonic energy to remove the tissue sample, and an aspirating pathway in communication with the tip. A mass spectrometry apparatus is in communication with the sampling probe via the aspiration pathway and configured to receive the tissue sample and analyze the tissue sample using a mass spectrometry process to generate mass spectrometry data. A computer system includes a computer processor having access to a non-transitory, computer-readable storage medium having stored thereon instructions that cause the computer processor to: receive the mass spectrometry data from the mass spectrometry apparatus, analyze the mass spectrometry data to determine a presence of at least one potential biomarker indicating the presence of cancer in the tissue sample, access a database of at least one of biomarker information and biomarker analysis algorithms, analyze the potential biomarker using the at least one of the biomarker information and biomarker analysis algorithms to determine a presence of cancer in the tissue sample, and determine from the presence of cancer in the tissue sample a likelihood of cancer in the tissue sample, and a report generator configured to deliver a report indicating the likelihood of cancer in the tissue sample.

In another aspect, the present invention provides a method for determining a presence of cancerous cells within a subject during a surgical procedure to remove the cancerous cells. The method includes harvesting the cancerous cells, positioning a sampling probe including an aspiration pathway proximate to an analysis site, vibrating a tip of the sampling probe in response to ultrasonic energy to remove a tissue sample, aspirating the tissue sample through the aspiration pathway, providing the tissue sample from the sampling probe to a mass spectrometry system, conducting a mass spectrometry procedure on the tissue sample to produce a spectrographic report, analyzing the spectrographic report to determine a presence of a biomarker indicating a presence of cancer in the tissue sample from the subject, and generating a report indicating a likelihood of cancer existing in the analysis site.

The foregoing and other aspects and advantages of the invention will appear from the following description. In the description, reference is made to the accompanying drawings which form a part hereof, and in which there is shown by way of illustration a preferred embodiment of the invention. Such embodiment does not necessarily represent the full scope of the invention, however, and reference is made therefore to the claims and herein for interpreting the scope of the invention.

### BRIEF DESCRIPTION OF DRAWINGS

The invention will be better understood and features, aspects and advantages other than those set forth above will become apparent when consideration is given to the following detailed description thereof. Such detailed description makes reference to the following drawings.

FIG. 1 shows a schematic of an exemplary system for determining a presence of cancer in a tissue sample within a procedure room.

FIG. 2 illustrates a type of probe that may be adapted for use in the present invention.

FIG. 3 is a drawing of a device that has a hand held base unit and an elongated metal rod.

FIG. 4 is an illustration of a hand held base unit for a probe.

FIG. 5 shows the terminal part of a device that includes an elongated metal rod terminating in an opening through which tissue samples may be aspirated.

FIG. 6 is a profiled spectra taken in a negative ion mode in accordance with the present invention using DESI-MSI.

FIG. 7 is a series of images taken in accordance with the present invention using DESI-MSI.

FIG. 8 is another series of images taken in accordance with the present invention using DESI MSI.

FIG. 9 is an averaged and normalized spectra of ions taken in the negative ion mode in accordance with the present invention using DESI MSI on the samples of Table 1.

FIGS. 10a and 10b illustrate a principal component analysis (PCA) of cases 9 and 14 using the software suite ClinProTools (Bruker Daltonics).

FIG. 11 is another series of images taken in accordance with the present invention using DESI-MSI.

FIG. 12 is another profiled spectra taken in the negative ion mode in accordance with the present invention using DESI-MSI.

FIG. 13 is another series of images taken in accordance with the present invention using DESI-MSI.

FIG. 14 shows negative ion mode DESI-MS mass spectra obtained in a linear ion trap mass spectrometer from m/z 100 to 1000 for samples G23, an oligodendroglioma with the IDH1 R132H mutant (a) and G31, a glioblastoma with wild-type IDH1 (b). Insets show zoom in region m/z 100-200.

FIG. 15 shows negative ion mode DESI-MS mass spectra obtained in a linear ion trap mass spectrometer from m/z 100 to 1000 for tandem mass spectra of m/z 147 detected from sample G42, an oligodendroglioma with the IDH1 R132H mutant (MS2, c; MS3, d) and 2-HG standard (MS2, e; MS3, f).

FIG. 16 shows plots of SNaPshot Mutation profiling of glioblastoma samples G28 and G33, both of which were not immunoreactive with the antibody that recognizes IDH1 R132H. The top panel shows genotyping data obtained with normal male genomic DNA (Promega, Madison, Wis.). The lower panels illustrate IDH1 R132C mutation detection in tumor DNA derived from formalin-fixed paraffin-embedded specimens of glioblastoma samples G28 and G33.

FIG. 17 shows negative ion mode DESI-MS images from sample G30, a glioblastoma with the IDH1 R132H mutation. The panels show the distribution of ions m/z 788.4, m/z 885.5, m/z 281.5 and m/z 147.2 (identified as 2HG). Optical images of R132H IHC and H&E stained tissue sections are shown.

FIG. 18 is a visualization of 2-HG levels over 3D-MRI volume reconstruction for samples A, B, C and D from surgical case 3.

FIG. 19 shows a tandem mass spectrum of m/z 147 detected from sample G31, a glioblastoma with wild-type IDH1.

FIG. 20 shows negative ion mode DESI-MS mass spectra obtained in a LTQ Orbitrap mass spectrometer from m/z 100 to 1000 for samples G42, an oligodendroglioma with the IDH1 R132H mutant (a) and G29, a glioblastoma with wild-type IDH1 (b). Insets show zoom in region m/z 146.90-147.16.

FIG. 21 shows mass spectrometry data indicating detectiion of 2-HG in gliomas using DESI MS.

FIG. 22 shows detecting 2-HG in glioblastoma with IDH1 R132G mutation.

FIGS. 23a show two-dimensional DESI MS ion images of human glioma resection specimen.

FIG. 23b shows a low-magnification light microscopy image of the glioma of FIG. 23a having been H&E stained.

FIG. 23c shows a higher magnification light microscopy image of the portion of the H&E stained glioma of FIG. 23b that is within the light grey box of FIG. 23b.

FIG. 23d shows a higher magnification light microscopy image of the portion of the H&E stained glioma of FIG. 23b that is within the black box of FIG. 23b.

FIG. 24 shows 3D mapping of 2-HG over MRI volume reconstruction for surgical case 10, an oligodendroglioma grade II and corresponding H&E stained tissue sections.

FIG. 25 shows an outline of the standard work flow for brain surgery in the AMIGO suite using current methodologies and the increased sampling that is possible with DESI-MS.

FIG. 25b shows immunohistochemistry using an IDH1 R132H point mutation specific antibody on formalin-fixed and paraffin embedded (FFPE) section from oligoastrocytoma grade II samples (S75), (scale bar, 100 μm).

FIG. 25c shows targeted mutational profiling using SNaPshot analysis on nucleic acids extracted from oligoastrocytoma grade II archival specimens (S75).

FIG. 25d shows high magnification light microscopy images of H&E stained swab (left), smear (middle) and frozen tissue section (right) are shown (scale bar, 200 μm).

FIG. 25e shows negative ion mode DESI mass spectra obtained using an amaZon Speed ion trap from m/z 130 to 165 (Bruker Daltonics, Billerica, Mass., USA) from a swab (left), a smear (middle) and a section (right) for sample S72.

FIG. 25f shows corresponding tandem mass spectra (MS2) of m/z 147.0 (left), 146.9 (middle) and 146.9 (right) detected from sample S72 present a fragmentation pattern that exactly matches that of standard 2-HG.

FIG. 25g shows normalized 2-HG signal is represented with a grey scale as indicated by the scale bar; set from the lowest (light grey) to highest (dark grey) levels detected from this individual case.

FIG. 26 shows images of H&E stained tissues, normalized 2-HG signals, and NIM-DESI mass spectra for case 28.

FIG. 27 shows a negative ion mode DESI mass spectrum from m/z 100 to 1000 for samples G31 and G23.

FIG. 28 shows a negative ion mode DESI mass spectra obtained in a LTQ Orbitrap mass spectrometer from m/z 100 to 1000 for samples G42, an oligodendroglioma with the IDH1 R132H mutant with 2-HG signal at m/z 147.0299 (a) and G29, a glioblastoma with wild-type IDH1 (b). Insets show zoom in region m/z 146.90-147.16.

FIG. 29 shows a normalization of 2-HG signal and estimation of limit of detection.

FIG. 30 shows a graph of normalized 2-HG signal versus tumor cell concentration in a glioma series with IDH1 mutation (see Table 1 for sample details).

FIG. 31 shows two-dimensional DESI MS ion images of human glioma cell xenografts in immunocompromised mice.

FIG. 32 shows two-dimensional DESI MS ion images of human glioma resection specimens.

FIG. 33 shows a 3D mapping of 2-HG over MRI volume reconstruction for surgical case 13, an oligoastrocytoma grade II.

FIG. 34 shows DESI-MSI lipid profiles of surgical samples D40 and D38. Negative ion mode mass spectra from GBM surgical sample D40 (A) and necrotic surgical sample D38 (B). Insets show optical images of the sections stained with H&E after DESI-MSI analysis. In the spectrum, m/z values were detected corresponding to lipid species exclusively detected in one of the two samples. In the spectrum, m/z values were detected corresponding to lipids species having a higher relative abundance in one of the two surgical samples.

FIG. 35 shows histological evaluation and DESI-MSI analyses of surgical sample D43.

FIG. 36 Spectral classification and PCA analysis from data acquired from DESI-MSI analysis of surgical sample D43.

FIG. 37 shows pLSA analysis from DESI-MSI analysis data from surgical sample D43.

FIG. 38 shows label-free 3D molecular imaging of tumor presentation with DESI-MS.

FIG. 39 DESI-MSI analyses of surgical samples D40 and D38. (A) H&E staining and DESI-MSI ion image representing the repartition of ion at m/z values 279.0, 391.3, 437.3 and 491.3 on surgical sample D40. (B) H&E staining and DESI-MSI ion image representing the repartition of ion at m/z values 544.5, 572.7, 626.6 and 650.6 on surgical sample D38.

FIG. 40 shows histological evaluation and DESI-MSI analyses of surgical sample D42.

FIG. 41 shows the spectral classification and PCA analysis from data acquired from DESI-MSI analysis of surgical sample D42. Additional m/z values are present in these two groups and imply that additional species could be specifically detected in GBM or necrosis tissue by DESI MS.

FIG. 42 shows pLSA analysis from DESI-MSI analysis data from surgical sample D42.

FIG. 43 is a flowchart showing a method according to aspects of this disclosure.

FIG. 44 is a flowchart showing a method according to aspects of this disclosure.

Table 1 is a summary of tissues samples used in exemplary experiments.

Table 2 is a detail of an exemplary bio marker.

Table 3 is a detail of another exemplary bio marker.

Table 4 is a detail of another exemplary bio marker.

Table 5 is a detail of another exemplary bio marker.

Table 6 is a detailed description of samples used in an IDH1 study. IHC and DESI results are shown, for both solvent systems used.

Table 7 shows 2-HG levels results for surgical Case 3.

Table 8 shows samples used in IDH1 study. IHC and DESI results are shown.

Table 9 shows classification results for samples from surgical case 9. Results indicate the percent of pixels within each image that were assigned to a given class. Surgical samples used as reference to build the SVM classifier are in boldface (D38 and D40). GBM, glioblastoma.

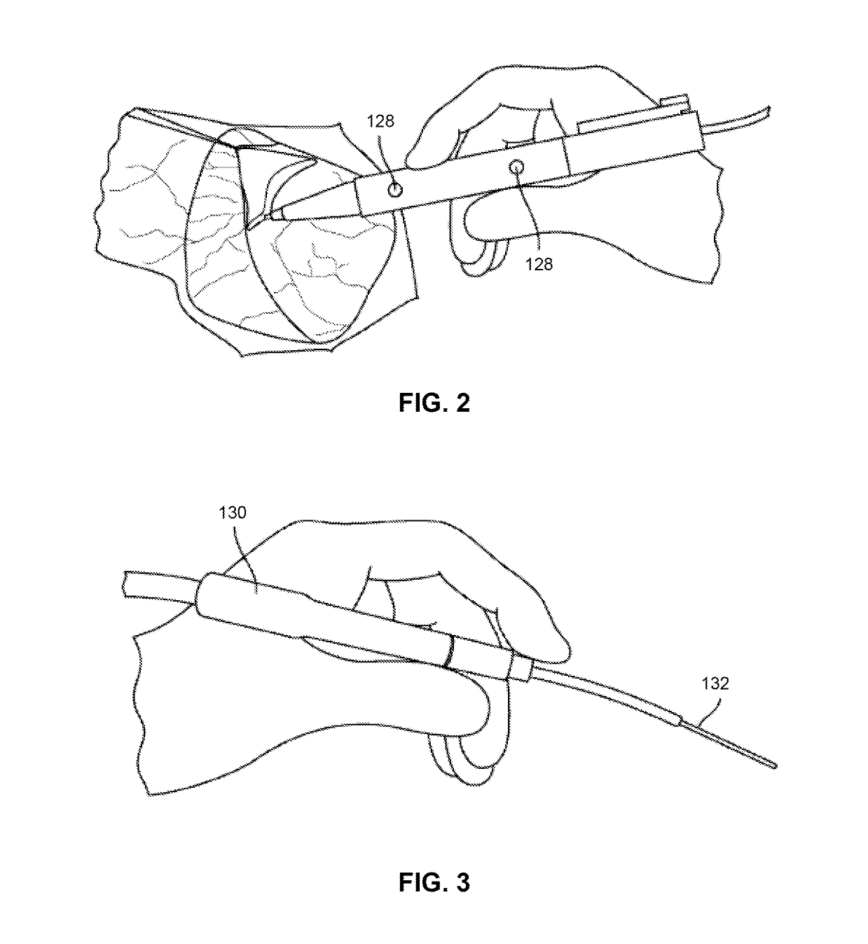

Table 10 shows p-values obtained for the eight peaks from t-tests. The p-values of the Wilcoxon/Kruskal-Wallis (PWKW) test and the Anderson-Darling Test (PAD) indicate a significant difference between the GBM and the necrosis data sets for each m/z value of FIGS. 2B and 2C (≦0.05 and >0.05, respectively). All the average intensity values for the m/z values 279.0, 391.3, 437.3 and 491.3 are also increased in the GBM average mass spectrum (Ave2 values) and the others (m/z values 544.5, 572.7, 626.6 and 650.6), in the necrosis average mass spectrum (Ave1 values). Index, sequence of peak; Mass, m/z; PTTA, p value oft-test (two classes).

While the invention is susceptible to various modifications and alternative forms, specific embodiments thereof have been shown by way of example in the drawings and are herein described in detail. It should be understood, however, that the description herein of specific embodiments is not intended to limit the invention to the particular forms disclosed, but on the contrary, the intention is to cover all modifications, equivalents, and alternatives falling within the spirit and scope of the invention as defined by the appended claims.

### DETAILED DESCRIPTION OF THE INVENTION

As discussed above, routine intra-operative distinction between tumor and normal breast tissue is currently not possible in breast conserving surgery. This limitation affects the success of many surgical procedures. For example, considering just one common cancer surgery, in breast cancer surgery, up to about forty percent (40%) of operations require more than one operative procedure.

Mass spectrometry imaging (MSI) has been applied to investigate the molecular distribution of proteins, lipids and metabolites without the use of labels. In particular, desorption electrospray ionization (DESI) allows direct tissue analysis with little or no sample preparation. Therefore, with the advantage of easy implementation, DESI mass spectrometry imaging (DESI-MSI) has great potential in the application of intra-operative tumor assessment. As described herein, imaging includes spatially encoded information correlated with the surgical site and/or the tissue histology itself. However, not all spectroscopy data in accordance with the present invention needs to be spatially encoded. For example, one or a series of points may be sampled with or without spatial encoding information and delivered to the clinician. Furthermore, when the spectroscopy data is spatially encoded, the spatial encoding may include two or three-dimensional spatial encoding. Thus, the data may be presented in pixels or voxels.

Mass spectrometry offers the possibility for the in-depth analysis of proteins and lipids comprising tissues. Desorption electrospray ionization-mass spectrometry (DESI-MS) is a powerful methodology for characterizing the lipids within tumor specimens. The ionization profile of lipids within tumors can be used for tumor classification and to provide valuable prognostic information such as tumor grade. Because DESI-MS is performed in ambient conditions with minimal pretreatment of the samples, diagnostic information can be provided rapidly within the procedure room. The present invention leverages the ability to quickly acquire such valuable diagnostic information from lipids to use DESI-MS to detect additional molecules of diagnostic value within tumors such as their metabolites.

Recurrent mutations have been described in the genes encoding isocitrate dehydrogenases 1 and 2 (IDH1 and IDH2) in a number of tumor types including gliomas, intrahepatic cholangiocarcinomas, acute myelogenous leukaemias (AML) and chondrosarcomas. These mutant enzymes have the novel property of converting α-ketoglutarate to 2-hydroxyglutarate (2-HG). This oncometabolite has pleiotropic effects impacting DNA methylation patterns, and the activity of prolyl hydroxylase activity. While 2-HG is present in vanishingly small amounts in normal tissues, concentrations of several micromoles per gram of tumor have been reported in tumors with mutations in IDH1 and IDH2. As will be described, the present invention enables the detection of, among other things, 2-hydroxyglutarate using 2-dimensional DESI-MS on a series of gliomas or other tumor types. Additionally, the invention may apply to other surgery situations outside of tumor boundary detection or to the recognition of other biomarkers. Detecting metabolites in tumor tissues with precise spatial distribution and under ambient conditions provides a new paradigm for intraoperative surgical decision-making.

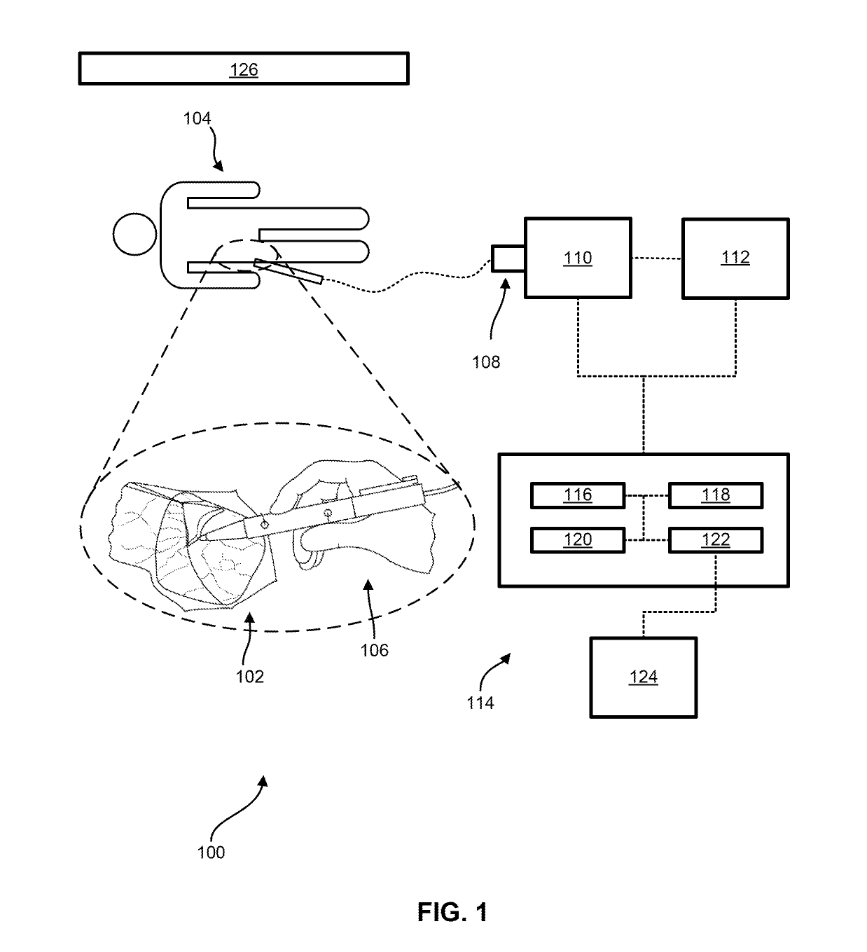

Turning to FIG. 1, a system 100 is provided in accordance with the present invention that is designed to analyze a sample 102 acquired from a subject 104, particularly during an operative procedure, such as may be performed in an operating room. The system 100 may be configured for use with a tool or probe 106 to assist or work in conjunction with other systems for providing the sample 102 to a sample receptacle 108 of the system 100. For example, it is contemplated that the system may be compatible with systems or method or include systems disclosed in co-pending U.S. patent application Ser. No. 13/059,524, which is incorporated herein by reference in its entirety. In some embodiments, the tool or probe can be surgical forceps or other similar apparatus that resects the sample 102 and provides the sample 102 to the sample receptacle 108.

Once a sample is provided to the sample receptacle 108, the sample is processed by a mass-spectrometry system 110. The mass-spectrometry system 110 analyzes the tissue to determine a presence of a biomarker indicating a presence of cancer in the tissue sample. The mass-spectrometry system 110 may be a desorption electrospray ionization apparatus. In any case, the mass-spectrometry system 110 is coupled to a report generator 112 that is configured to deliver a report indicating a likelihood of cancer remaining in the subject based on the analysis and, more particularly, the above-described biomarkers. The report generator 112 may include a printing system to print a physical report or may include a display to display a report, including figures and user-interface components, for example, such as will be described with respect to FIGS. 2-9 and those derived therefrom.

The mass-spectrometry system 110 and/or report generator 112 may include or be connected to a computer system 114. The computer system 114 includes a computer processor connected to a non-transitory, computer-readable storage medium or memory 118 that can store computer programs to control operation of the computer system 114 and, thereby, control operation of or coordinate operation with the mass-spectrometry system 110 and/or report generator 112. Accordingly, the computer system 114 may include any of a variety of user interfaces 120 or communications mechanisms, including a keyboard, mouse, touch screen, monitor, audio or video input or output, and the like. In addition, the computer system 114 may include a variety of input or communications connection 122, including traditional computer-system input/outputs, network communications ports (wired and wireless) that may provide access to wide and local networks and the Internet. By way of the communications connection 122, the computer system 114 may be coupled to a database 124 or other information repository. As will be described, the database 124 may store a variety of information to facilitate data analysis, including data on various biomarkers, such as will be described, and various algorithms or processes that the processor 116 may utilize to analyze information about the sample provided to the receptacle 108 and provide a report through the report generator 112.

Thus, in operation, the system 100 can be utilized within an operating room or any clinical setting that would benefit from accessing tissue information to support a clinical decision to provide real-time feedback to a surgeon or other clinician. In addition to the mass spectrometry results and the feedback regarding any of a variety of biomarkers and/or analysis algorithms, the probe 106 may also be coupled with additional navigation or recording systems, such as disclosed in co-pending U.S. patent application Ser. No. 13/059,524.

That is, the probe 106 may include stereotactic tracking elements or beacons that are linked to imaging components. In one construction, an imaging device 126 such as an MRI, CAT, CT, PET, MRS, or other imaging device is used to create a three-dimensional (3D) anatomical image of the surgery site. The stereotactic tracking elements may then be used to track the probe 106 or the location from which a tissue sample was manually resected within the anatomical image. In this way, the surgeon may track the location within an additional image of where the tissue sample was collected and correlate the report details, such as the spectroscopy images, to the exact location. In this way, the surgeon may use the 3D image as a map and examine various areas of the surgery site for the presence of biomarkers, for example through a report generator 112. Regardless of whether additional imaging or tracking systems are used, the system 100 provides the surgeon with real-time and direct feedback about the operating site. This provides a very powerful tool for real-time feedback during medical procedures. For example, in the case of a cancer resection, the system 100 allows the surgeon or clinician to completely remove the cancerous cells, while maintaining the maximum amount of healthy tissue intact, because, as will be described, the feedback from the system 100 can indicate the presence or absence of cancer cells in real-time.

The report generator 112 is located within the procedure room such that the surgeon can monitor the anatomical image, the probe location, and the spectroscopy data and image for any sampled point within the surgery site in real time. This provides the surgeon with more information about the surgery while he or she can still affect the outcome of the surgery without having to wait for lengthy lab procedures. The report generator 112 may include a visual monitor that includes a color display large enough to be easily read in an procedure room environment. The display may be large enough such that it is easily read to reduce error of interpretation during surgery. The display can provide the anatomical image, the spectroscopy data and images, the stereotactic tracking information, and other information related to the surgery as desired. The Figures show several examples of the type of information which may be displayed on the report generator 112. The report generator 112 can be configured to be mounted within the procedure room.

Thus, using the invention, the surgeon or other clinician can verify the full resection of the cancer while still in the procedure room.

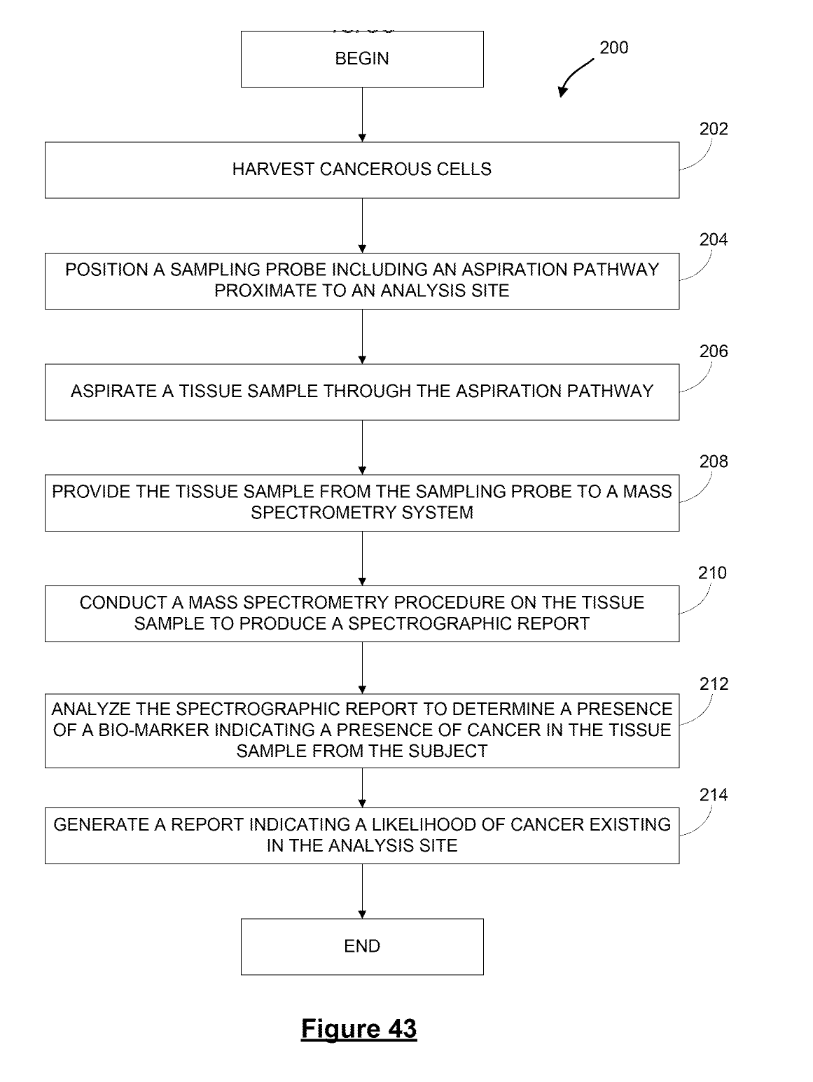

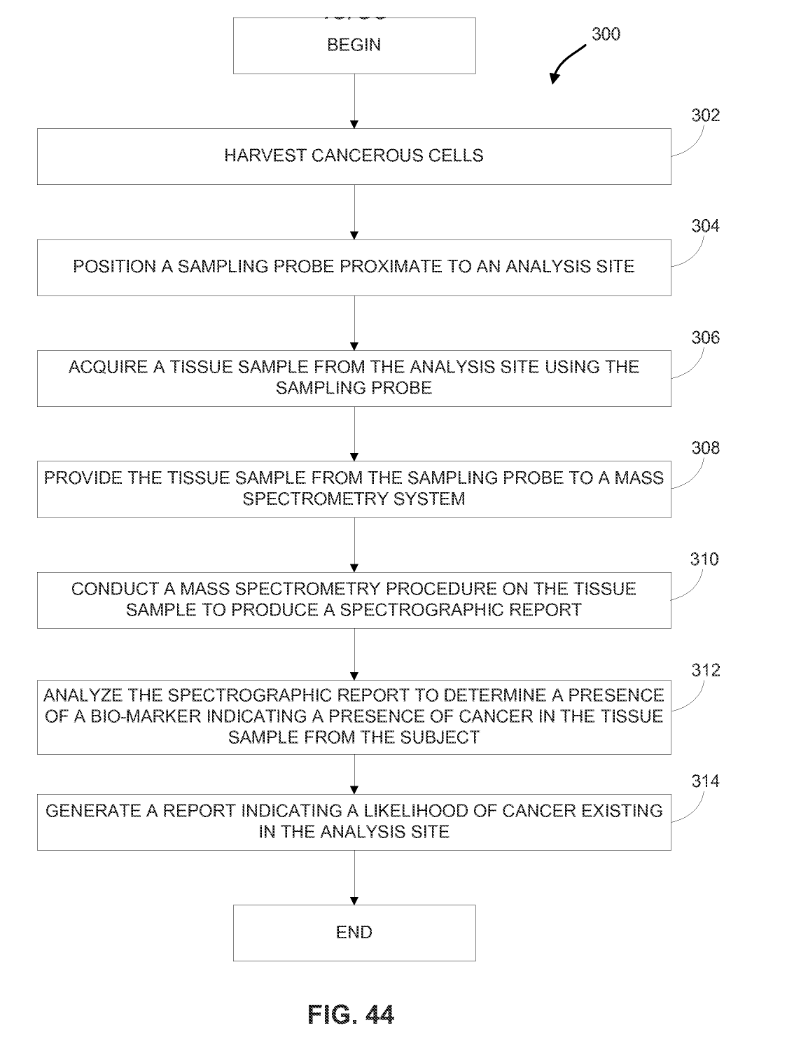

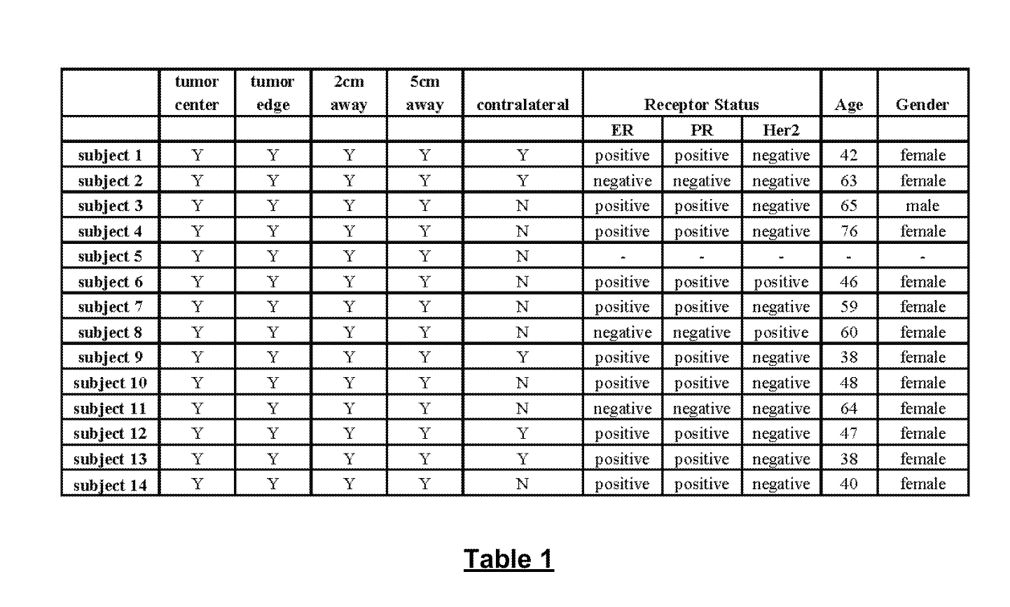

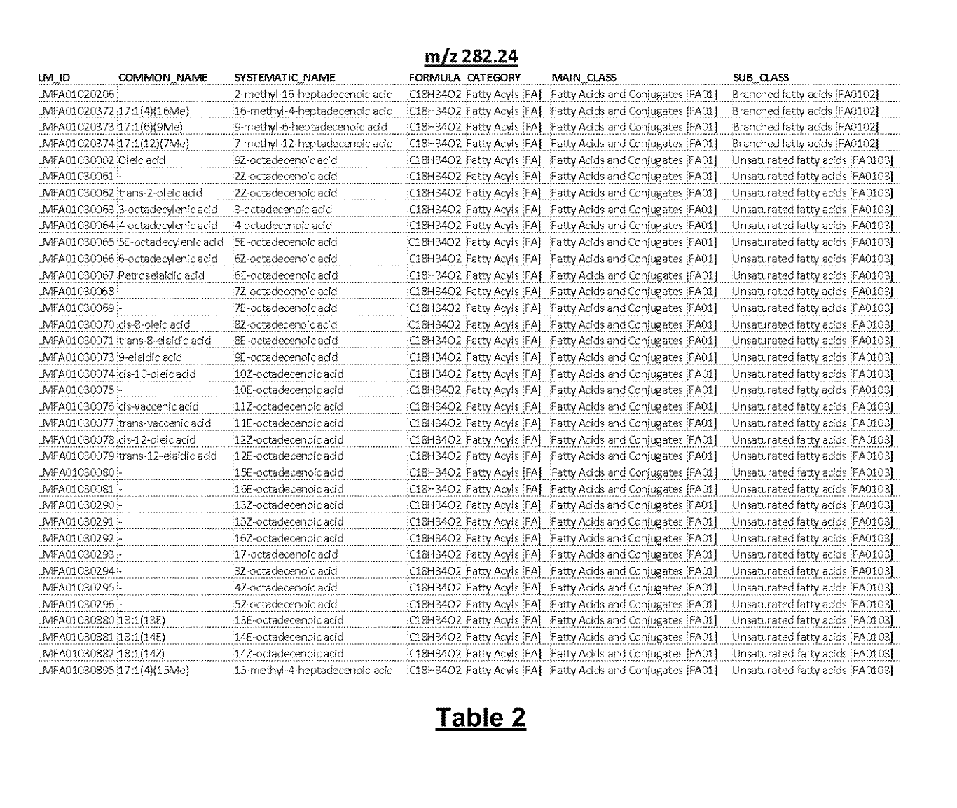

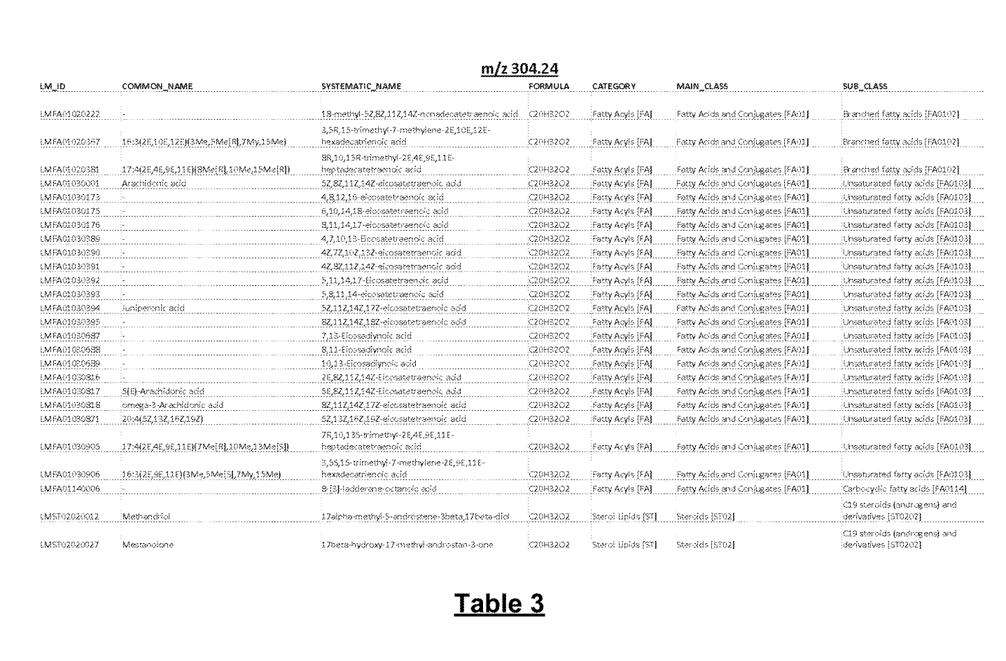

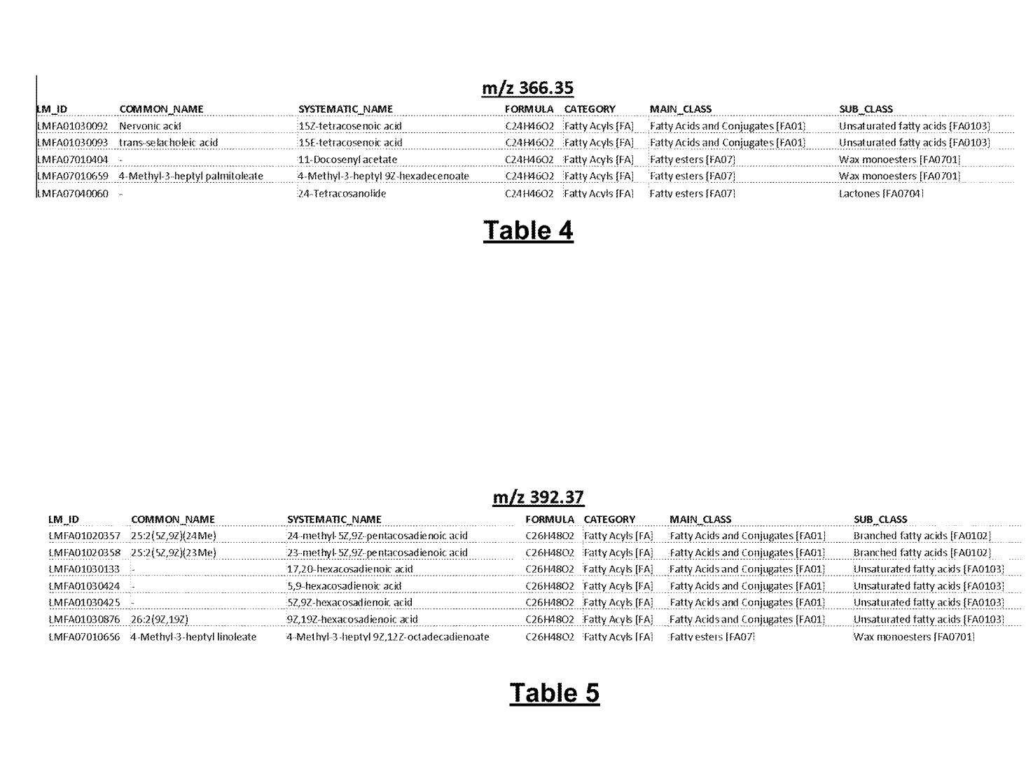

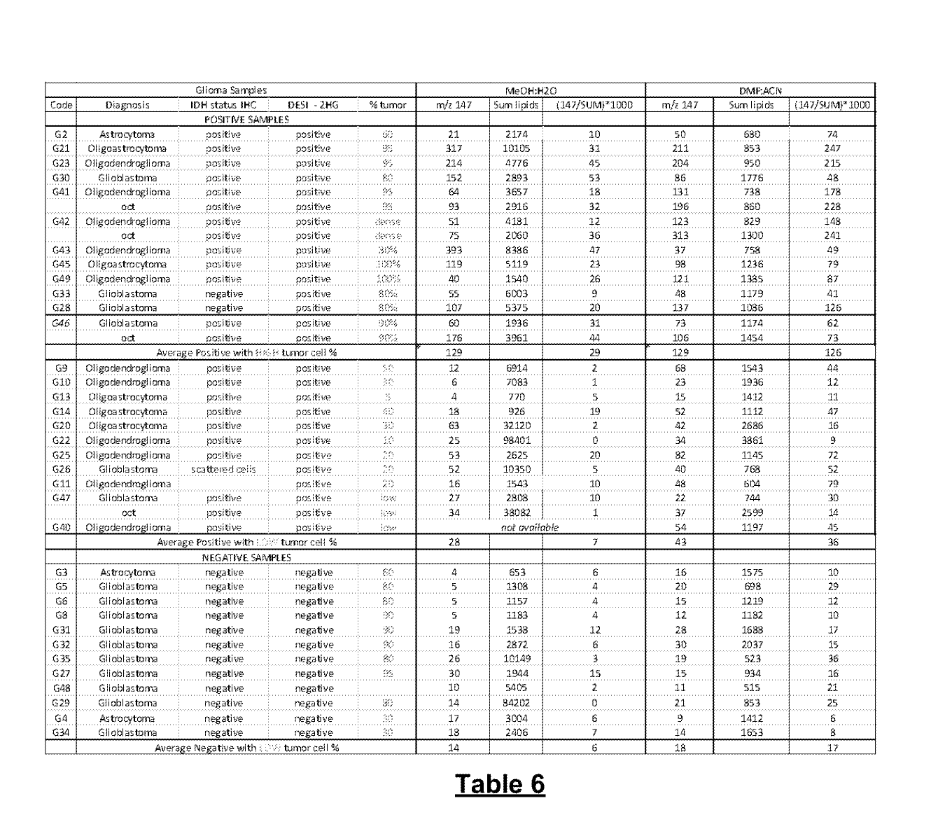

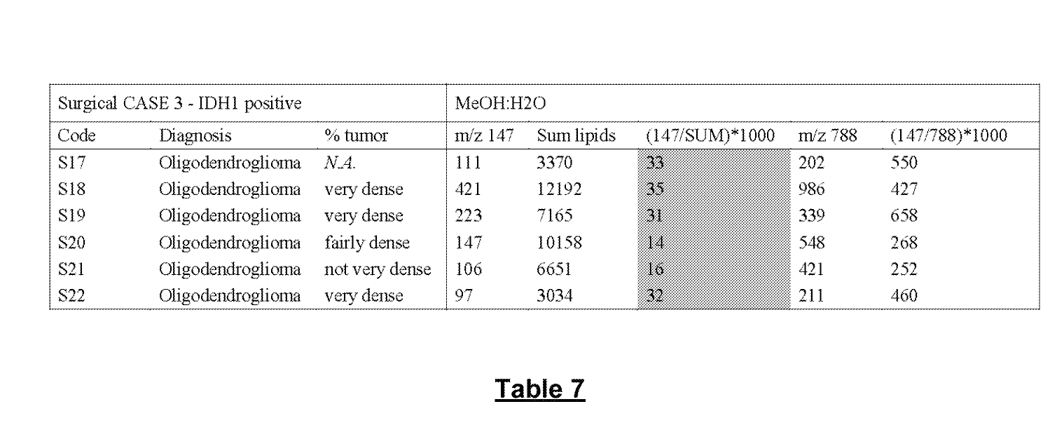

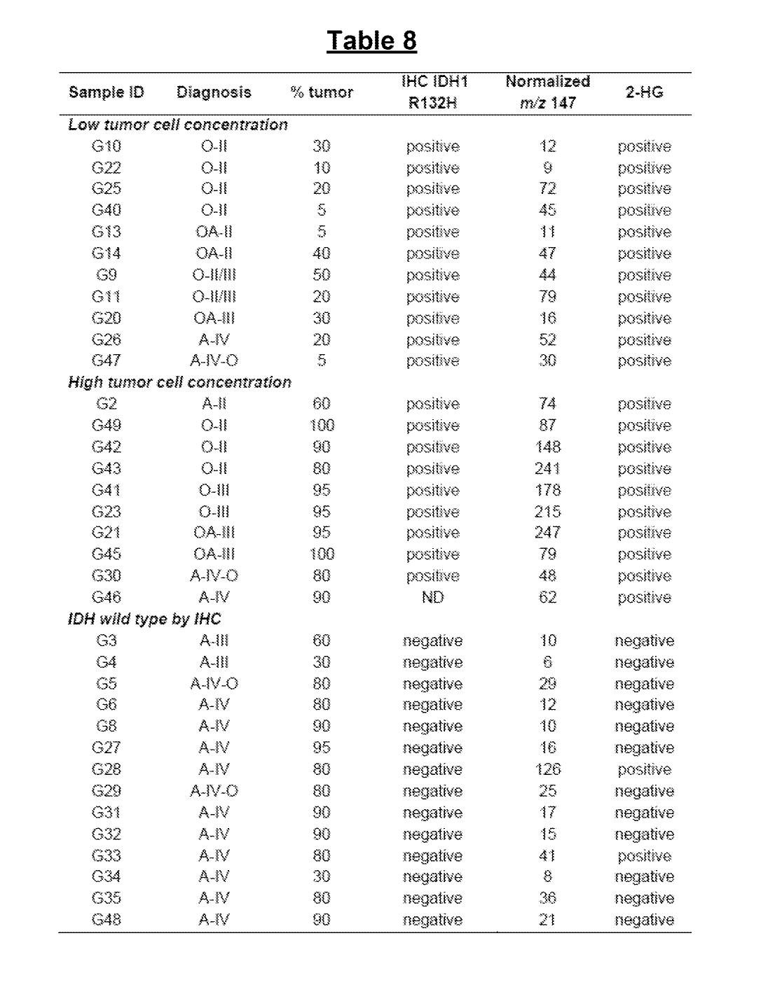

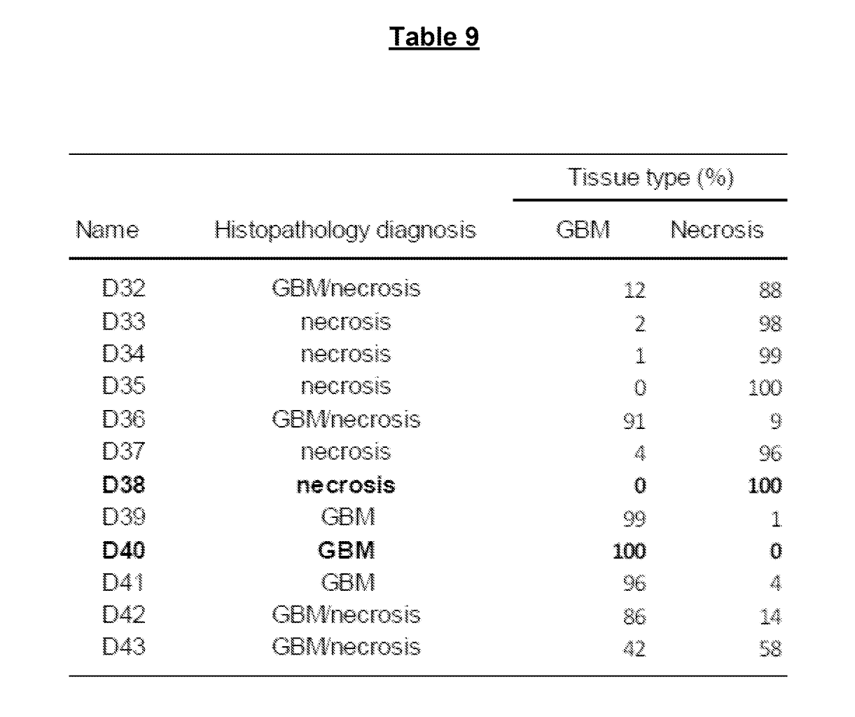

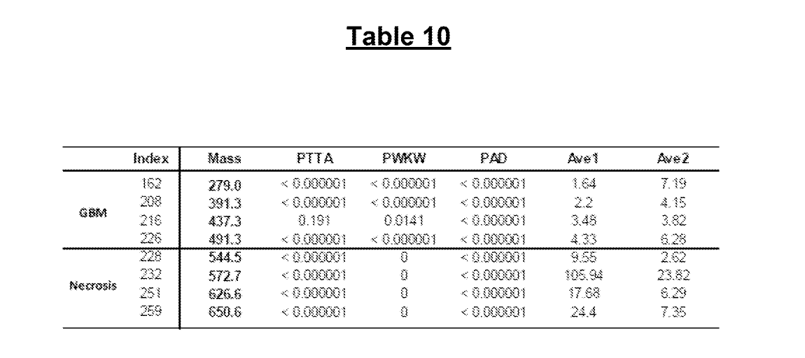

Referring to FIGS. 43 and 44, flowcharts illustrate methods 200, 300 for determining a presence of cancerous cells within a subject during a surgical procedure to remove the cancerous cells using the approach described herein. Additionally or alternatively, the following process may be performed to analyze a margin, for example, of a resected sample of tissue, without requiring the sample to be sent to a pathology lab located remotely and wait for results to be returned after the surgical procedure has ended. Rather, such a sample or margin may be analyzed contemporaneously with the surgical procedure that harvested the sample.

The method 200 may begin at process block 202 where a harvesting of cancerous cells is performed. As one non-limiting example, this may be an interventional or surgical procedure, for example, performed in a procedure room. At process block 204, a sampling probe including an aspiration pathway is positioned proximate to an analysis site. For example, the analysis site may be a location in the subject that was proximate to the harvested cancer cells, such that an in vivo analysis can be performed. As another example, the analysis site may be a portion of a resected or harvested sample, such that an in vitro analysis can be performed. As will be described, in either case, the present disclosure provides a system and method to perform the following analysis to provide a report that can be used to inform further clinical decisions with respect to a surgical or cancer removal procedure. As an alternative, the sampling probe can be a tool that mechanically resects a sample and provides it to a mass spectrometry system.

Next, at process block 206, a tissue sample is aspirated through the aspiration pathway. Subsequently, at process block 208, the tissue sample is provided from the sampling probe to a mass spectrometry system. Next, at process block 210, a mass spectrometry procedure is conducted on the tissue sample to produce a spectrographic report. At the following process block 212, the spectrographic report is analyzed to determine a presence of a biomarker indicating a presence of cancer in the tissue sample from the subject. Finally, at process block 214, the method includes generating a report indicating a likelihood of cancer existing in the analysis site.

The method 300 may begin at process block 302 where a harvesting of cancerous cells is performed. At process block 304, a sampling probe is positioned proximate to an analysis site. At process block 306, a tissue sample is acquired from the analysis site using the sampling probe. At process block 308, the tissue sample is provided from the sampling probe to a mass spectrometry system. At process block 310, a mess spectrometry procedure is conducted on the tissue sample to produce a spectrographic report. At process block 312, the spectrographic report is analyzed to determine the presence of a biomarker indicating a presence of cancer in the tissue sample from the subject. At process block 314, a report is generated indicating a likelihood of cancer existing in the analysis site.

In certain embodiments, the computer processor is further caused to determine a relative abundance of the biomarker. In certain embodiments, the report generator is configured to indicate a higher relative abundance of the biomarker as compared to healthy tissue as indicating cancer in the tissue sample. In certain embodiments, the step of analyzing can include determining a relative abundance of the biomarker. In certain embodiments, the relative abundance of the biomarker is higher in a cancerous tissue sample than a healthy tissue sample.

In certain embodiments, the report can include a chart of a relative abundance of all detected ions. In certain embodiments, the method further include indicating a boundary between cancerous cells and non-cancerous cells using the report.

In certain embodiments, the mass spectrometry apparatus or procedure can include a desorption electrospray ionization. In certain embodiments, the mass spectrometry apparatus or procedure can include operating in a negative ion mode or a positive ion mode.

In certain embodiments, aspirating the tissue sample can include providing irrigating fluid to a tip of the sampling probe through the irrigation channel.

In certain embodiments, the method can further include vibrating a tip of the sampling probe in response to ultrasonic energy to remove the tissue sample.

In certain embodiments, the method can further include conducing an imaging procedure. The method can also include stereotactically tracking a location of the tip of the sampling probe. The method can further include correlating the report to the tracked location of the tip within an image produced by the imaging procedure. The imaging procedure can include, among other things, a magnetic resonance imaging procedure, an ultrasound imaging procedure, and the like.

In certain embodiments, the biomarker includes a lipid. In certain embodiments, the biomarker includes one of m/z 89.1, m/z 281.3, m/z 282.24, m/z 303.3, m/z 304.24, m/z 365.4, m/z 366.35, m/z 391.4, m/z 392.37, m/z 413.4, m/z 445.4, m/z 572.6, m/z 626.8, m/z 656.8, and m/z 682.8.

The following description of the operation and features of the system 100 is divided into five sections. SECTION I discusses an exemplary probe 106 for obtaining the tissue sample and tracking the location of origin of the sample. Section II discusses various details an exemplary methodology of sample acquisition and imaging in accordance with the present invention. SECTION III illustrates a second exemplary methodology of sample acquisition and imaging in accordance with the present invention. SECTION IV illustrates a third exemplary methodology of sample acquisition and imaging in accordance with the present invention. SECTIONS II and III discuss the use of laboratory techniques for secondary analysis of the collected samples. The laboratory analysis was conducted as a way to verify the invention's effectiveness and to verify the effectiveness of the inventive methodology and system. Thus, the discussion of the methods for validation utilize traditional analysis techniques/systems, rather that the system of FIG. 1. Of course, when not reliant upon traditional methods and systems for purposes of validation, the underlying systems and methods can be readily performed, for example, using a system such as described above with respect to FIG. 1. That is to say, going forward, the mass spectrometry analysis that is performed in the procedure room would be sufficient thereby providing the advantages of real-time feedback to the surgeon in the procedure room. SECTION V provides one example of a user of the invention in an operating room setting.

Section I

One example of a probe 106 that integrates a tissue resection device with a stereotactic navigation system and uses the device to collect tissue fragments for diagnostic assays is discloses below. The probe 106 allows tissue sampling locations to be precisely determined. Preliminary results and published articles reporting on the histopathological evaluation of tissue fragments indicate that ultrasonically generated fragments preserve the features required for standard histopathological diagnosis. It is expected that mechanically generated fragments would also preserve these features.

In one aspect, the probe 106 includes a medical device that can be used in collecting tissue samples from biopsy sites in a patient. The device includes a hand held support, also referred to as a hand held base unit, typically made of plastic, metal or rubber with a shape and size that allows it to be easily held and maneuvered in an operator's hand. Typically, these supports will have a rectangular or cylindrical shape and be about 4 to 8 inches in length, although other shapes and sizes are possible. Extending from, and attached to, one end of the hand held support is an elongated metal rod with a proximal end (the end attached to the support) and a distal end (the end furthest from the support). The rod will typically be about 3 to 10 inches long and terminate at its distal end in a tip that either itself vibrates in response to ultrasonic energy or which has a separate component attached to it that vibrates in response to ultrasonic energy. Alternatively the tip may include a sharpened cutting surface that, in response to electrical stimulation, cuts or shaves tissue.

The medical device also includes means for supplying ultrasonic energy to the tip or to the separate vibrational component, preferably at a frequency of 15-100 kHz and, more preferably, at 20-60 kHz. Alternatively the device may be designed to respond to the input of electrical energy by moving in a manner that results in the cutting or shaving of tissue. For example, there may be an electrical motor that causes the tip to rotate in the manner of a drill in response to electrical input.

In addition, the device includes means for supplying irrigating fluid to the distal end of the tip and for aspirating tissue fragments created at the tip as the result of ultrasonic vibrations or due to mechanical cutting or shaving. A preferred method for supplying irrigating fluid is by pumping it from a reservoir through a tubular channel running through the rod and terminating in an opening at the tip. The exact diameter of the channel is not critical to the invention but will typically be between ⅛ and ½ of an inch. The reservoir may contain any pharmaceutically acceptable fluid such as water, saline, Ringer's solution etc. and may be maintained at room temperature or chilled, e.g., to 0-15° C. If desired, the fluid may also include antibiotics to help prevent infection or other drugs.

With respect to aspiration, it is preferred that the metal rod of the device have a hollow core that provides a fluid passageway for tissue fragments. This passageway is open at the tip and extends through or past the hand held support of the device. Sufficient suction is provided, e.g., by means of a medical suction pump, to aspirate material through the opening at the tip in the direction of the hand held support. As with the channel for providing irrigation fluid, the diameter of the passageway for aspiration is not critical but will, in general, be between ⅛ and ½ inch.

The passageway may be connected at its proximal end to a tissue collection container where aspirated fragments are delivered and which, in some embodiments, contains a fluid such as water, saline or Ringer's solution. This fluid may, optionally be chilled, e.g., to 0-10° C., and may include chemicals for fixing tissue samples. In an alternative embodiment, the tissue fragments may be delivered to a container in which they are quick frozen, e.g., in dry ice or liquid nitrogen. The collected sample may alternatively be supplied directly to a mass spectrometry device without the use of a storage container or solution. In the event that a storage container or solution is used, the storage container or solution will typically not require extensive preparation or lab work such that the tissue sample may be collected and supplied to the mass spectrometry device within the procedure room without the need for additional laboratory work or preparation.

The probe 106 provides a system for stereotactically determining the position of the distal end of the rod (i.e., the location where aspirated tissue samples are collected) relative to the tissue being examined (e.g., brain tissue).

The stereotactic system may include a computer that stores information regarding the spatial relationship between the probe (particularly the tip of the probe) and the tissue of the patient being examined. The probe includes means for communicating information to the computer regarding its position. This may be accomplished using, inter alia: a) ultrasound detectors; b) electromagnetic emitters located on the device (preferably on the hand held support of the device) that transmit signals to a separate electromagnetic receiver; c) sound emitters located on the device (preferably on the hand held support) that transmit signals to microphones; d) by optical tracking using infrared energy detectors; or e) other stereotactic tracking devices, as desired. In each instance, signals are communicated to the computer for analysis. The most preferred method for communicating information concerning the position of the device is with electromagnetic emitters.

In one embodiment, the hand held support includes an actuator switch which, when activated, permits the transmission of ultrasonic or mechanical energy to the tip of the rod or to a separate component which vibrates in response to ultrasonic energy. When the switch is not activated ultrasonic energy is not transmitted. Activation of the actuator switch is, preferably, accompanied by the transmission of a signal to the computer to aid in determining the position of the tip of the rod at the time of actuation. In an alternative and more preferable design, the actuator switch is in the form of a foot pedal which, when activated, transmits ultrasonic energy to the tip of the rod. Actuator switches may also be used which, instead of causing rod tips to ultrasonically vibrate, cause the tip to move in a manner that results in the cutting or shaving of tissue.

In another aspect, the probe 106 provides a method of collecting a tissue sample from a biopsy site by inserting the tip of any of the medical devices described above into a patient so that the distal end of the rod is positioned at the site where the biopsy is to be performed. Energy is then transmitted to the tip of the rod to create ultrasonic vibrations at the site and fragment tissue or to cause the cutting or shaving of tissue. Irrigation fluid is then administered at the biopsy site and the fragments are aspirated into a collection container where they are retrieved for histological examination or other diagnostic tests.

The probe 106 may be used in the system 100 for methods of collecting tissue samples that are mapped to a particular biopsy site. The first step in these methods is to establish a three dimensional stereotactic coordinate system for reproducibly identifying positions in the tissue that is to be examined, e.g., a portion of a patient's brain or a tumorous growth. Any of the stereotactic positioning systems described in the various references cited above can be used for this purpose. Next, one or more diagnostic imaging procedures (e.g., a CT or MRI scan) are performed to identify areas in the patient where one or more biopsy samples may be taken. Finally, tissue samples are collected using a medical device that fragments the tissue, collects the fragments that have been generated and records the position of sampling in the stereotactic coordinate system. The information obtained using this procedure will be particularly useful when multiple sites are sampled, for example, to determine how far cancer cells have invaded. Although the methodology can, in principle be applied to any site in a patient's body, it is expected that, initially, it will be most useful for biopsies involving brain tissue or breast tissue.

FIGS. 2-5 show an exemplary probe 106 in the form of a hand held device.

Devices of this type can incorporate a component into the hand held support that will provide a signal that can be used in analyzing its exact position. For example, electromagnetic emitters may be included in the support to provide a signal to a separate receiver, which, in turn, communicates this information to a computer for analysis. A drawing of a probe 106 with electromagnetic emitters 128 is shown in FIG. 2. The use of electromagnetic sensors in place of the electromagnetic emitters 128 or a combination electromagnetic sensor and emitter is contemplated. Other signaling systems that may be used include those that detect ultrasonic signals, sound signals and infrared signals, among others. In certain embodiments, the probe disrupts cellular tissue through longitudinal vibration of a hollow tip at ultrasonic frequencies (24 or 35 kHz). Combining the disruption process with irrigation coming from the annular space surrounding the probe assists in removal of the tissue by aspiration through the center of the handpiece into a waste container. For the purposes of the present invention, the waste container is replaced with a collection container and/or analytical device. FIG. 2 shows two electromagnetic emitters 128 or sensors that have been located on the hand held support of the device for signaling its position.

Referring to FIG. 3, the probe 106 can include a hand held base unit 130 with an elongated metal rod 132 extending from it. In certain embodiments, the elongated metal rod 132 may be curved.

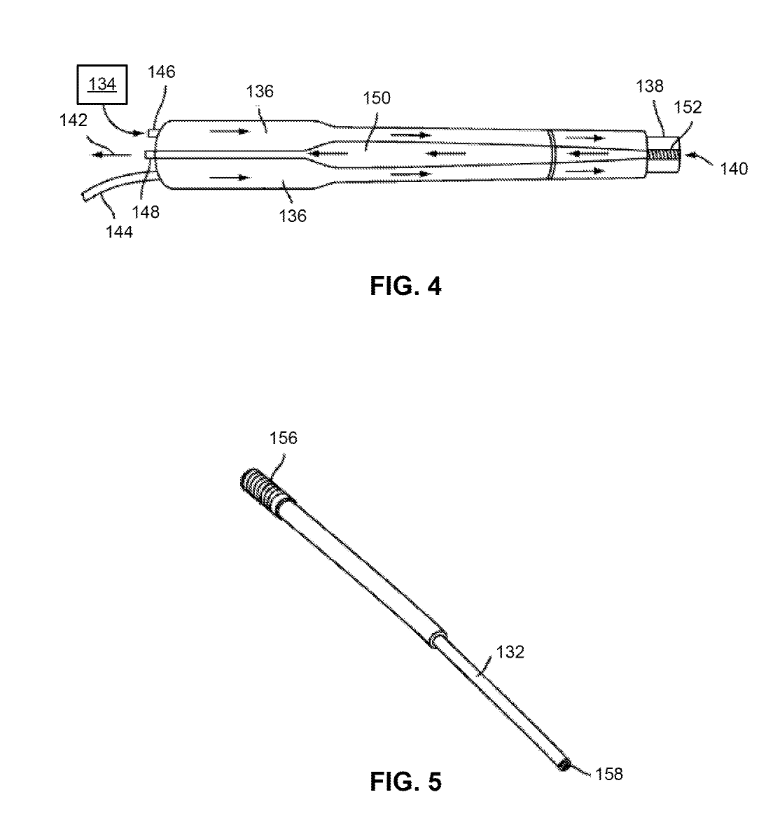

Devices should also include means for irrigating and aspirating tissues after fragmentation. This is illustrated in FIG. 4 which shows the hand held base unit 130 of a device and FIG. 5 which shows the terminal part of a probe that includes an elongated metal rod 132. As shown in FIG. 4, the hand held base unit 130 can include a reservoir 134 containing irrigation fluid that is connected to a port 146 leading into a channel 136. The channel 136 runs to the distal end of the hand-held base unit 130 where fluid exits and flows or sprays onto tissue. The distal end of the hand held base unit 130 includes a coupling region 138 that attaches to the elongated rod 132 (shown in FIG. 5) which vibrates at its tip in response to ultrasonic energy provided by an ultrasonic energy generator and transmitted via a cord 144. The coupling region 138 can include a threaded bore 152. The distal end of the base unit 130 has an opening 140 that leads into a passageway 150 extending from the opening 140 to a port 148 at the opposite end of the base unit 130. Aspirated tissue exits this port 148 in a stream 142 and may be delivered to a collection container. This container may contain fluids such as water or saline to preserve the tissue fragments and may optionally be chilled or contain fluid for fixing tissue. Samples recovered from the collection container may then be examined for histological features characteristic of disease or used in other diagnostic tests. Referring to the embodiment shown in FIG. 5, the elongated rod 132 is designed to attach to the handheld base unit 130 (not shown in FIG. 5) by means of a threaded region 156 at its base that can be screwed into a matching threaded bore (152 in FIG. 4) in the handheld support.

In order for the devices described above to provide information on the location of sampling sites, they should be integrated with existing systems for the stereotactic analysis of spatial arrangements. The first step in using these systems is to establish a three dimensional stereotactic coordinate system for reproducibly identifying positions in the tissue of the patient. This is usually accomplished using an apparatus or electrodes that are placed in fixed positions on the patient as a frame of reference. Diagnostic imaging procedures (e.g., CT scans or MRI scans) may then be performed to provide information concerning the internal tissues of the patient and the spatial relationship of the tissues to the established coordinate system. For example, imaging procedures may be used to provide information on the exact location of a tumor. After imaging, an important step is the registration step which takes place in the OR.

The final step is to use the medical devices described herein to obtain tissue fragments while, at the same time recording the exact position where each sample was collected. The sample from each site is retrieved from the device and diagnostically analyzed. In this way, pathologic differences in a tissue may be determined. For example, different sites from tissue containing a tumorous growth may provide information on areas in need of surgical resection and sections that can be spared. This is particularly important in tissues such as the brain where as much normal tissue as possible must be preserved.

In one exemplary use, brain tumor specimens were collected using both surgical forceps and CUSA, and then mass spectrometry analyses were performed. A validating histopathological analysis showed preservation of histology features required for diagnosis, and the direct mass spectrometry analysis of the tissue specimens using a DESI-LTQ instrument revealed molecular signatures indicative of neoplasia, as compared to specimens biopsied using surgical forceps. This new integrated surgical-sampling probe can enable the differentiation of tumor from non-tumor tissue based on measurements or imaging of the samples.

Section II

Methodology:

Tissues Sample Preparation:

During development of the invention, Applicants obtained sixty-one (61) cancerous breast samples removed via mastectomy from fourteen (14) research subjects from Brigham and Women Hospital. The samples (shown in Table 1) were collected at a tumor center, a tumor edge, 2 cm away from the tumor edge, 5 cm away from the tumor edge, and from a contralateral breast when available. The types of breast cancer were classified based on the status of three most important receptors: estrogen receptor (ER), progesterone receptor (PR) and human epidermal growth factor receptor 2 (Her2). Among the fourteen cases, nine of them have the tumor type ER positive, PR positive and Her2 negative (ER/PR+, Her2−), which is the most commonly found in breast cancer. As to the gender, one male was included.

Without the above-described system fully developed to allow real-time analysis, samples were flash frozen and stored in −80° C. freezer prior to analysis. The tissues were sectioned at 12 μm thickness using Microm HM550 crystat (Mikron Instrument Inc). 20 μm thickness was selected in several cases with fatty tissue. All the samples were mounted on regular glass slides. The slides were dried in a dessicator before analysis.

DESI Mass Spectrometry Imaging:

All the samples were analyzed using AmazonSpeed mass spectrometer (Bruker Daltonics, MA) connected with a commercial DESI source (Prosolia Inc., IN). The stage holding the glass slides mounted with tissue sections moved horizontally at the speed of 200 μm/s and vertically by 200 μm step to generate 2D image. The stage movement was controlled by OminiSpray 2D (Prosolia Inc., IN). A nondestructive solvent containing 50% acetonitrile and 50% dimethylformamide was used. A flow rate of 1 μL/min was selected for the solvent spray. The spectra were acquired within the mass range m/z 50-1100 with Bruker software Hystar (Bruker Daltonics, MA). In order to display 2D image, FireFly (Prosolia Inc., IN) was used to convert the data to be compatible with Biomap. All the images obtained from Biomap were displayed with the same intensity scale in each figure.

Histological Staining:

Standard hematoxylin and eosin staining (H&E Staining) was performed on the same tissue section after DESI MS imaging as well as serial sections to visualize tissue morphological information. Glass coverslips were used to cover slides with toluene in between as mounting medium. All the reagents used for H&E staining were purchased from Sigma (Sigma-Aldrich, St. Louis, Mo.). The optical tissue images were scanned using Axio Imager M1 microscope (Zeiss, Chester, Va.) at 40× magnification. The morphology of tissue sections was evaluated on the Mirax Digital Slide Desktop Server system.

Results:

Lipid Profiling in Breast Cancer Tissues Using DESI-MSI in Negative Ion Mode:

As discussed above, tissue samples from a total of fourteen research subjects with various ages were analyzed using DESI-MS imaging. All the samples were analyzed in a negative ion mode. The spectra were collected within the range of m/z 50-1100. Therefore, the negatively charged ions from lipids and metabolites were acquired. To validate day-to-day reproducibility, mouse brain sections were tested in exactly the same condition at the beginning of the day before acquiring breast cancer data.

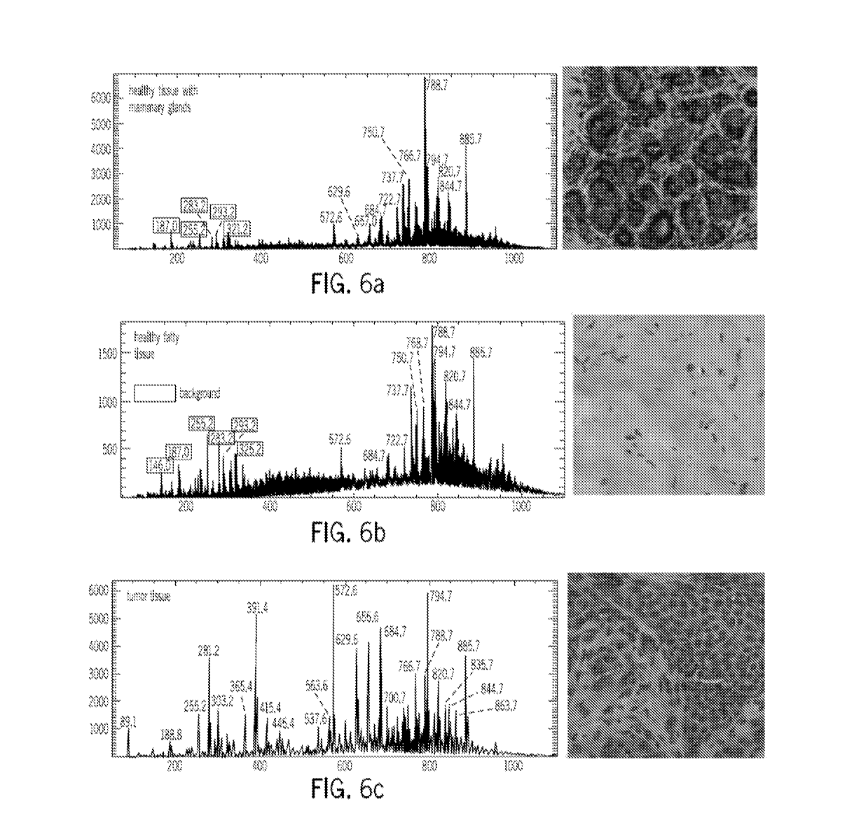

The representative profiled spectra from breast cancerous and healthy tissue sections are shown in FIG. 6 with corresponding optical images after histological staining DESI-MS analysis followed by standard H&E staining was performed on the same tissue sections. The nondestructive spray solvent containing 50:50 ACN/DMF was used in the experiment and the tissue integrity was preserved after DESI-MS, allowing the subsequent histological analysis. The feasibility of evaluating these H&E stained tissues was approved by a breast pathologist. In healthy tissue from mammary glands and normal fatty tissue, similar lipid ion species and relative abundance (e.g. PS18:0/18:1 with m/z 788.7 and PI18:0/20:4 with m/z 885.7) were observed (FIGS. 6a and 1b), whereas the signals in fatty tissue were less intense. The dominant ions from healthy tissues were within the mass range m/z 700-1000. It can be concluded that these lipids came from noncancerous cells and higher intensities were obtained from mammary glands only because of the high cell density. However, distinct lipid species and intensities were observed in the profiled spectrum from breast cancer tissue, especially in low mass region (FIG. 6c). Distinctive fatty acid ions were detected in low mass range <m/z 400 and lipids around m/z 600 were more abundant in cancerous tissues. In contrast, only background peaks were observed in low mass range <m/z 500 in healthy tissue in FIGS. 6a and 1b.

Based on the profiled spectra, significantly distinct lipids were detected from breast cancer and normal cells. Distinctive peak patterns in low mass region were observed in tumor tissue. However, the tissues from tumor edge, depending on cancer cell concentration, gave varying relative abundance in low mass range of the profiled spectra.

DESI-MSI of Breast Cancer Tissues in Negative Ion Mode:

DESI-MSI was performed on the breast cancer samples to display two-dimensional images correlating the lipid intensities with spatial distributions. Chemical information combining with tissue morphology is able to confirm the differentiation of tumor and healthy tissue based on molecular images from DESI-MSI.

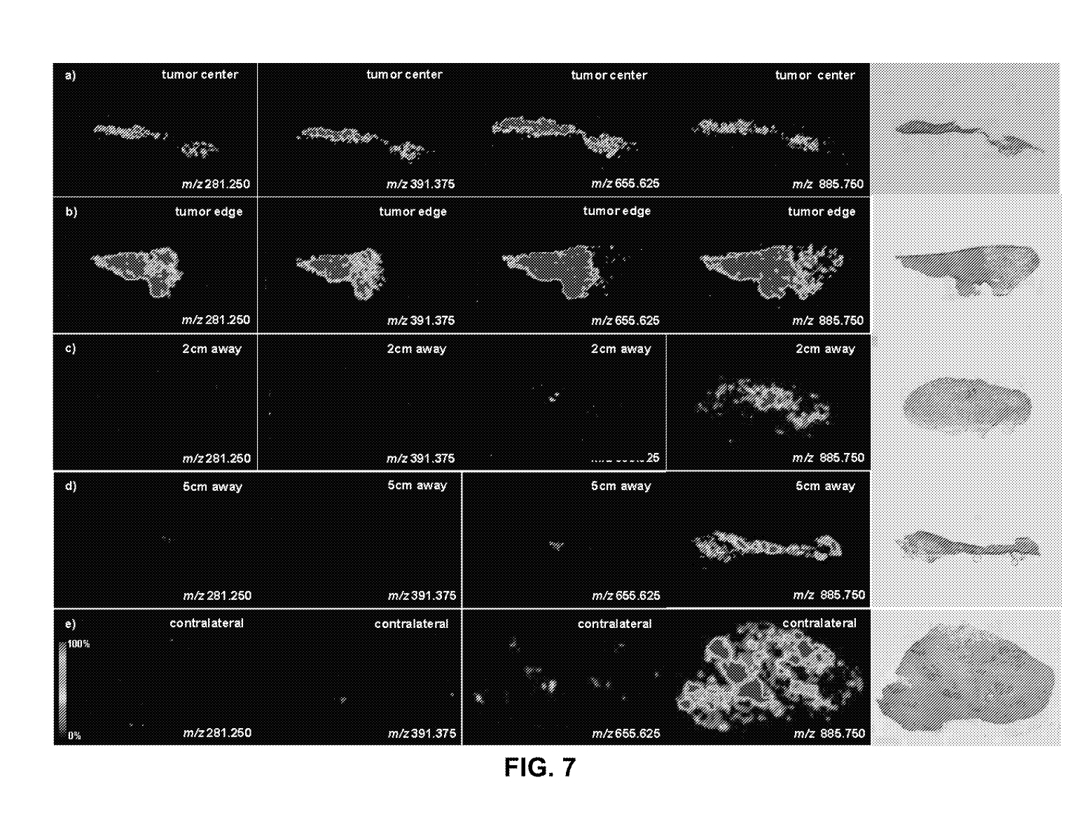

FIG. 7 includes the DESI-MSI images from samples of a tumor center, a tumor edge, 2 cm away and 5 cm away from the tumor edge, and a contralateral breast of research subject #9 respectively. Four ions, m/z 281.250 (oleic acid), m/z 391.375, m/z 655.625 and m/z 885.750 (PI18:0/20:4), were selected as representatives. All these images were plotted with the same grey scale. The lipid PI18:0/20:4, present in both healthy and cancerous cells, was used as a control to state successful ion detection. Evidently, PI18:0/20:4 is more abundant in the areas with mammary glands and tumor. The DESI-MS images from healthy tissues (2 cm away, 5 cm away from tumor and contralateral side) were highly consistent with mammary gland distributions stained by H&E staining of the same sections. However, distinct images were observed for ions with m/z 281.250, m/z 391.375 and m/z 655.625. These lipids were very abundant in the tumor center, where there was high tumor cell density (FIG. 7a), whereas these lipids were absent or weak in healthy tissues (FIGS. 7c, 2d and 2e). Interestingly, in the tissue section from the tumor edge the tumor margin was significantly delineated by the ion images of m/z 281.250, m/z 391.375 and m/z 655.625, which agreed well with the one demonstrated by the histological staining of the same section. The ion with m/z 655.625 was still present although very weak in normal cells.

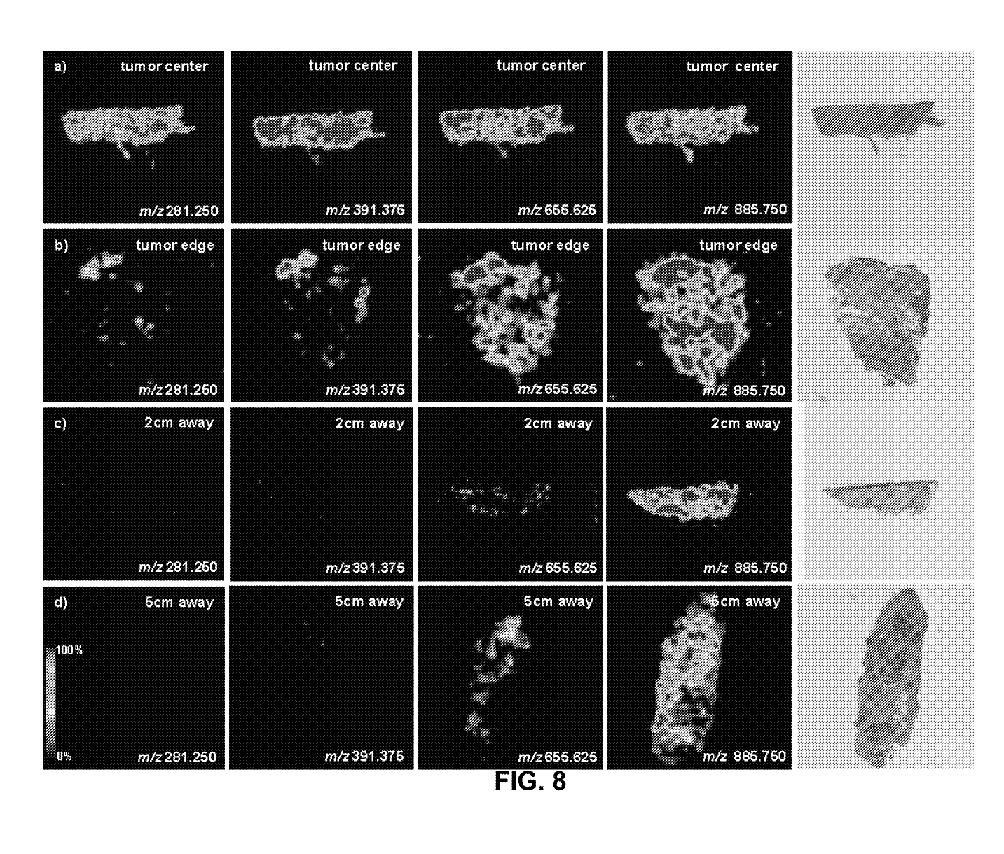

Another example from research subject #14 is shown in FIG. 8. Similarly, the ions with m/z 281.250 and m/z 391.375 were abundant in the tumor center (FIG. 8a), but absent in healthy tissues from 2 cm away and 5 cm away from the tumor (FIGS. 7c and 2d). The ion with m/z 655.625 was less intense but still observed in normal tissues with similar distributions as mammary glands in normal tissues. Interestingly, the DESI MSI of these ions were distinct in the tumor edge. In contrast with the ion with m/z 655.625, m/z 281.250 and m/z 391.375 were abundant only on the edge of tissue.

Tumor and normal tissues were able to be distinguished unambiguously based on single molecular image of certain lipid obtained from DESI-MSI. Overall 12 out of 14 cases demonstrated striking difference for ion images with m/z 281.250 and m/z 391.375 between tumor and healthy tissues. The use of nondestructive solvent with 50/50 ACN/DMF allows the subsequent histopathological evaluation on the same section as the tissue integrity was retained. The tissues from the tumor edge revealed distinctive molecular images but consistent with the tumor cell distributions evaluated by breast pathologist, allowing the delineation of tumor margin. The results establish the possibility of incorporating DESI-MSI intra-operatively for rapid diagnosis of breast cancer tissue.

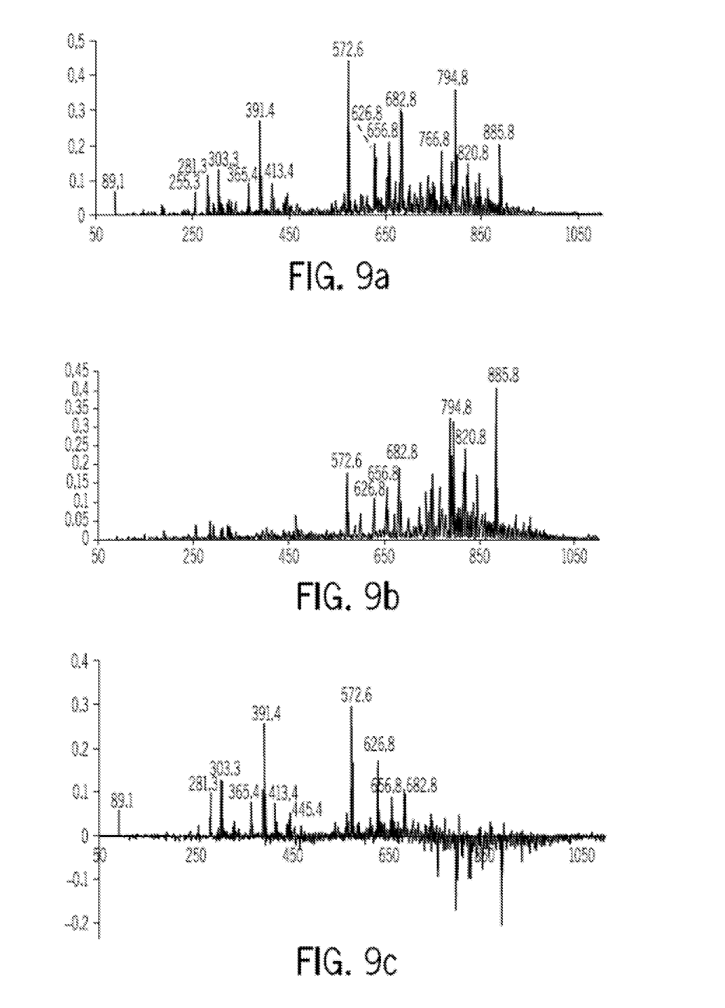

A typical spectrum to represent unique peaks only from tumor cells can be obtained by subtracting the ions coming from normal cells from the ions coming from tumor as shown in FIG. 9. The average of 13 and 14 spectra from the tumor and normal tissues respectively are displayed in FIGS. 9a and 9b with the subtracted spectrum shown in FIG. 9c. The ion intensities were normalized before the subtraction. While the lipid abundance was decreased dramatically in the mass range >m/z 700 after subtraction with some even having negative intensity e.g. m/z 885.8, the representative peaks from tumor were significant in the subtracted spectrum, especially in low mass region. This distinctive subtracted spectrum can be used in the statistical analysis in the future to guide the intra-operative identification of tumor tissue.

Principal Component Analysis:

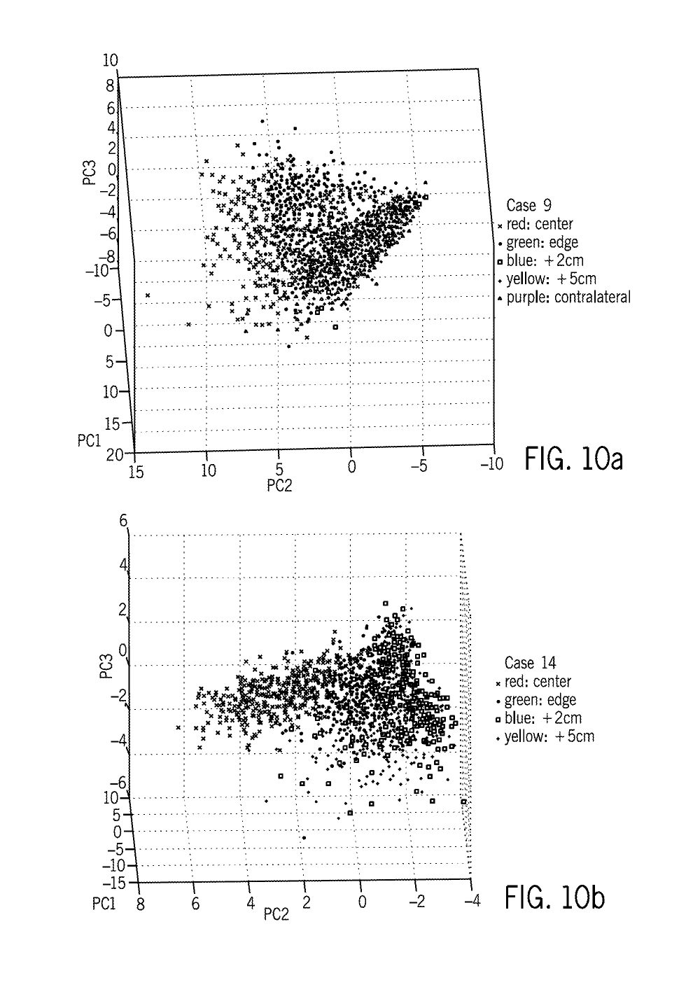

Although the tumor tissue can be differentiated from healthy tissue simply according to single molecular image from DESI-MSI, principal component analysis (PCA) was conducted for more accurate evaluation using ClinProTool. The statistical analysis of data from research subject #9 and #14 were shown in FIGS. 10a and 10b, respectively. In particular, FIG. 10a shows case 9 samples representing normal signatures such as contralateral breast, 5 cm and 2 cm away, clustered together, while the tumor edge and tumor samples derive from the normal cluster. Individual points each represent a spectrum (pixel) from the samples, and the samples harboring tumor derive from normal in a gradient suggesting an infiltrating edge or heterogenous composition of the tissue. FIG. 10b shows case 14 spectra from the tumor edge clustered between normal and cancerous sample. Separation of the spectra from the tumor and normal tissue was observed in both cases. In FIG. 10a, the spectra from tumor edge of research subject #9 were mostly clustered with spectra from tumor, whereas those from research subject #14 in FIG. 10b the tumor edge spectra were clustered between normal and cancerous sample. A combine analysis of both cases shows an overlap between tumor and tumor edge of both cases, and a clustering of 2 cm away, 5 cm away, and contralateral.

Abnormal Observation of Oleic Acid:

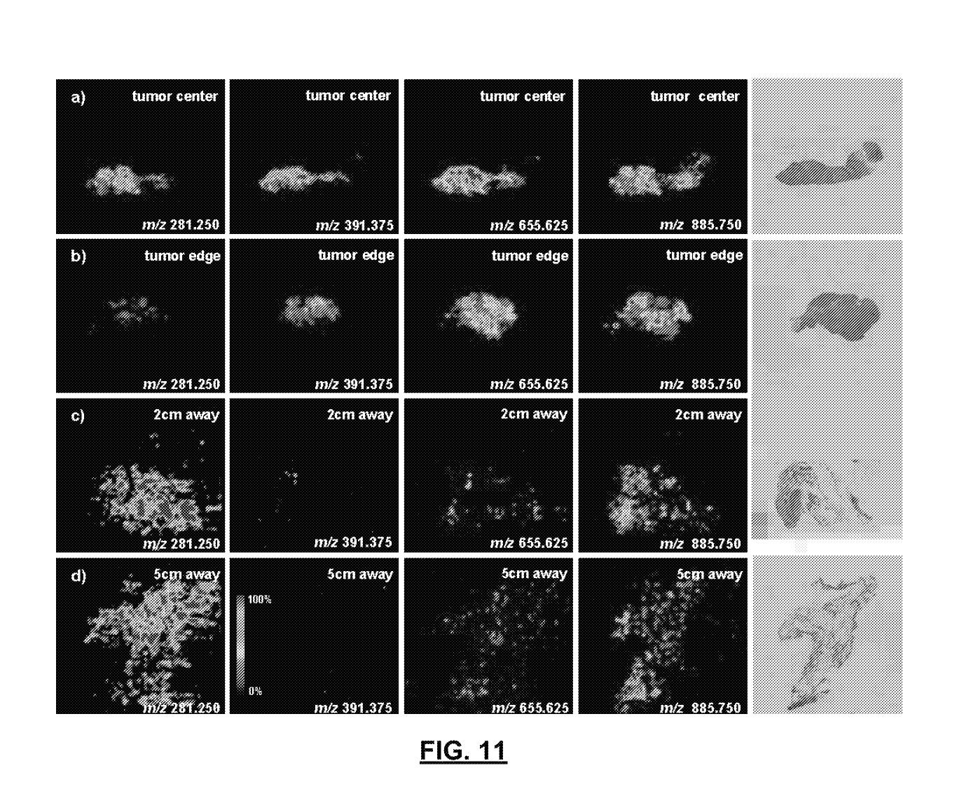

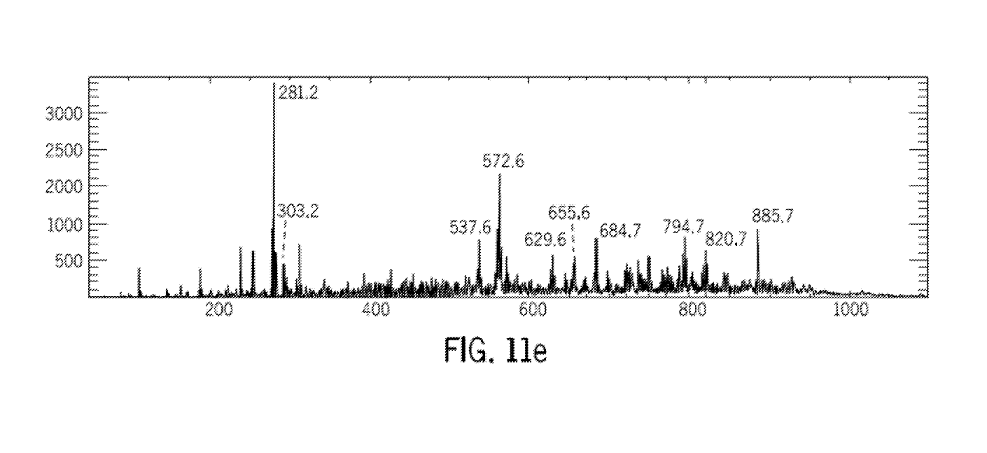

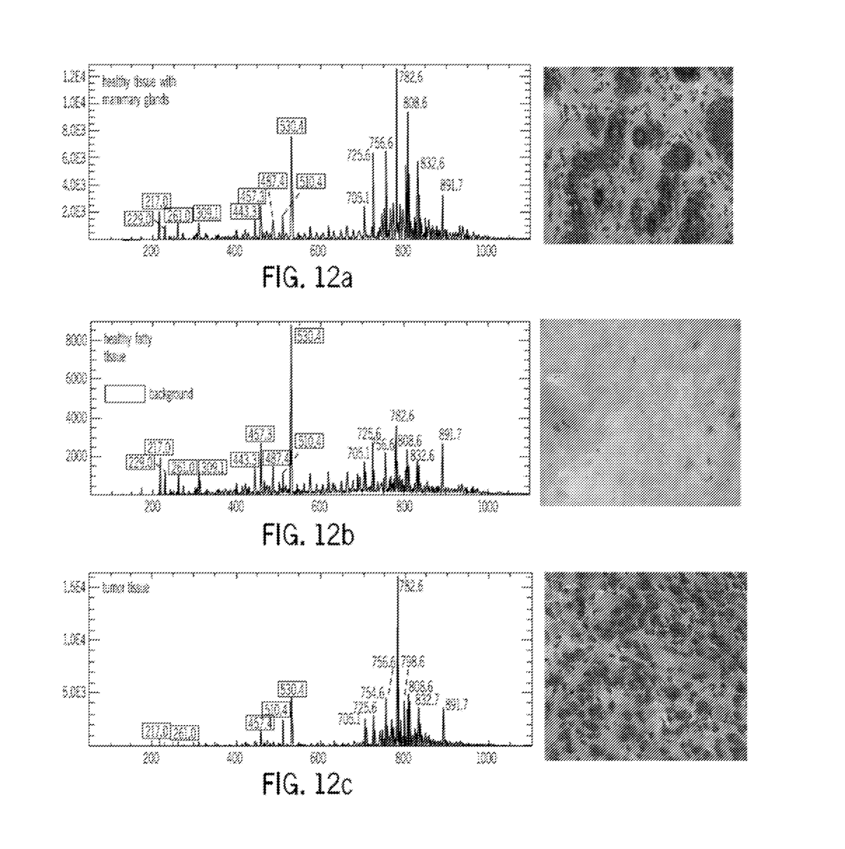

An interesting phenomenon was observed in research subject #5 that oleic acid signals (m/z 281.2) in normal tissues were increased dramatically (FIGS. 11c and 6d) compared with the tumor center and the tumor edge (FIGS. 11a and 6b), while the ions with m/z 391.375 and m/z 655.625 remained with low intensity. The dominance of oleic acid in the profiled spectrum of normal tissue is obviously visualized in FIG. 11e. Serial sections were analyzed repeatedly using DESI-MSI and similar results were obtained.

Lipid Analysis of Breast Cancer Tissues in Positive Ion Mode:

The tissue sections from normal and tumor samples were also analyzed using DESI-MSI in positive mode. The representative spectra are shown in FIG. 12. The same lipid species were observed in both tumor and normal tissues, mostly PC and SM lipids. Similar to the negative ion mode, the healthy tissue with mammary glands gave more abundant signals compared to the normal fatty tissue (FIGS. 12a and 7b). However, in the profiled spectrum from the tumor tissue, the relative abundance of m/z 782.6 to other ions was dramatically changed (FIG. 12c). The ion images obtained by DESI-MSI failed to exhibit the discrimination between tumor and mammary glands in normal tissues with similar cell density. However, the incorporation of unique lipid relative abundance in the tumor is able to improve the confidence of detecting cancer tissue based on MS analysis.

Discussion:

A mass spectrometry based methodology is demonstrated here to distinguish breast cancerous and noncancerous tissue in order to potentially facilitate breast surgeon's decision making intra-operatively. Samples from 14 research subjects acquired at various locations of breast with tumor were investigated. The application of DESI-MSI enables the differentiation of the tumor from normal tissues and determination of a tumor boundary based on molecular images.

Compared with positive ion mode, the lipid spectra obtained from negative ion mode gives more unique information. In the profiled spectrum from negative ion mode, distinctive fatty acids and lipids were identified in breast cancer tissues. About 85% of the samples showed a significant increase of ion abundance in the low mass region (<m/z 700) in tumor samples, while most ions in high mass range (e.g. m/z 885.7) exist in normal cells as well. A “tumor” spectrum can be obtained by subtracting the ions coming from normal tissue, which represents the unique ions from cancer and facilitates tumor tissue diagnosis using mass spectrometry. In 2D images from DESI-MSI, the distinction of cancer and healthy tissue can be directly visualized. The tumor margin was able to be delineated even based on single molecular image validating the DESI-MS based diagnosis of breast cancer. Statistical analysis was performed to confirm the classification of tumor and normal tissues.

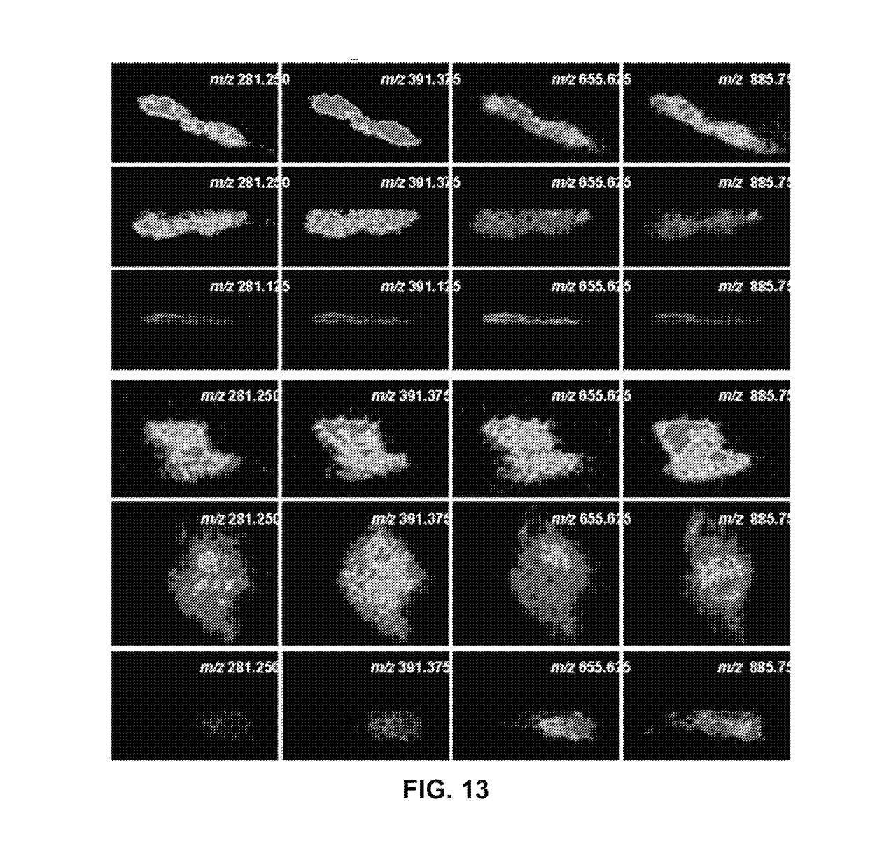

It is known that the lipids in breast samples degrade quickly during defrosting. In the exemplary experiments discussed above, although the samples were transferred carefully from −80° C. freezer to −20° C. cryostat for sectioning, the dramatic decrease of lipid signals were observed in DESI-MSI when the tissues were resectioned. The comparison of the tissues from the same sample but sectioned and analyzed at different days is shown in FIG. 13, where the top row and fourth row were analyzed on a first date, the second row and fifth row were analyzed on a second date 11 days after the first date, and the third row and bottom row were analyzed on a third date 115 days after the first date. Obviously, the lipid ions were much less abundant on the third date. Therefore, in order to obtain reliable lipid information, it is important to retain the samples fresh before analysis.

Tables 2-5 show details of a number of biomarkers that may be useful for identifying tumor margins or boundaries. These biomarkers were uncovered during Applicants' study of DESI-MSI analysis. Further, the following biomarkers were uncovered in the negative ion mode:

| MARKER | CHEMICAL FORMULA |
|---|---|
| m/z 89.1 | TBD |
| m/z 281.3 | C18H34O2 |
| m/z 303.3 | C20H32O2 |
| m/z 365.4 | C24H46O2 |
| m/z 391.4 | C26H48O2 |
| m/z 413.4 | . . . |
| m/z 445.4 | C30H53O2⁻ |
| m/z 572.6 | . . . |
| m/z 626.8 | . . . |
| m/z 656.8 | . . . |
| m/z 682.8 | . . . |

The markers represented above and in Tables 2-5 are examples only. Other markers may exist and would be detected by the inventive system and method. In addition, all chemical formulas, names, identifications, and classifications are exemplary and form no boundary about the invention.

Section II

Identification of 2-Hydroxyglutarate by DESI-MS

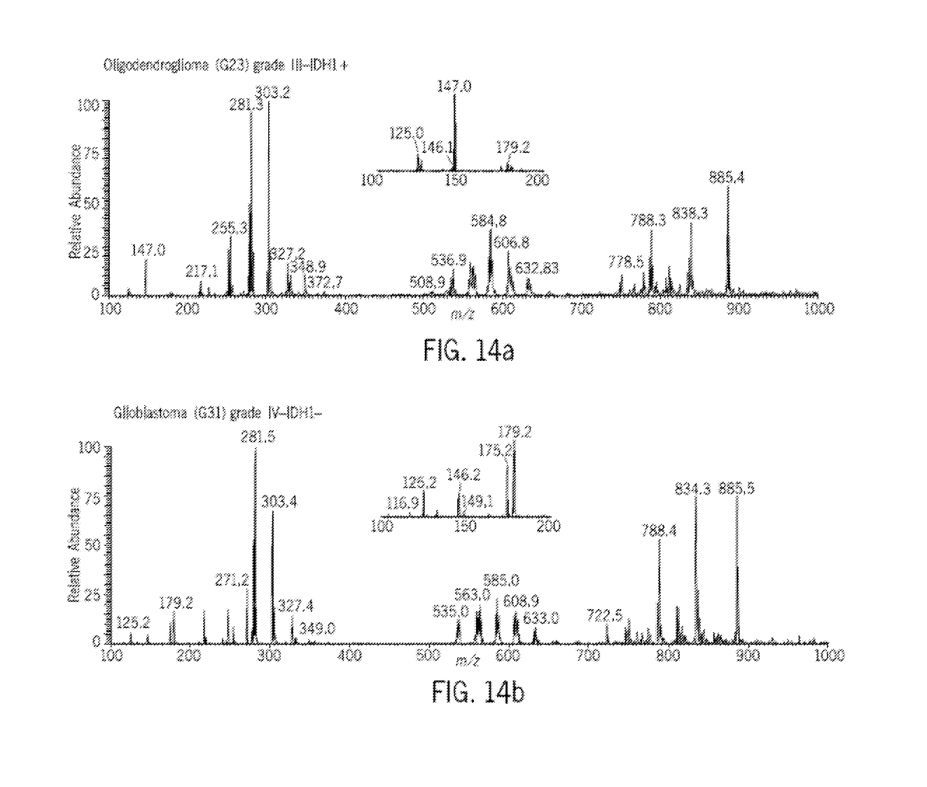

To determine if 2-hydroxygluterate (2-HG) could be detected from glioma tissue sections by DESI-MS, the negative ion mode mass spectra were first collected from two glioma samples: an oligodendroglioma with mutated IDH1 (encoding the amino acid change R132H) and a glioblastoma with wild-type IDH1. 2-HG is a small organic acid containing two carboxylic acid functional groups in its structure. In the negative ion mode, in its deprotonated form, 2-HG should be detected at an m/z of approximately 147. Together with the rich diagnostic lipid information commonly observed from gliomas by DESI-MS in the mass range m/z 200-1000, an intense peak was detected at m/z 147.2 in an IDH1 mutated sample (FIG. 14a), but not in an IDH1 wild-type sample (FIG. 14). A much less intense peak at approximately noise levels was observed at m/z 147.1 for the IDH1 wild-type sample.

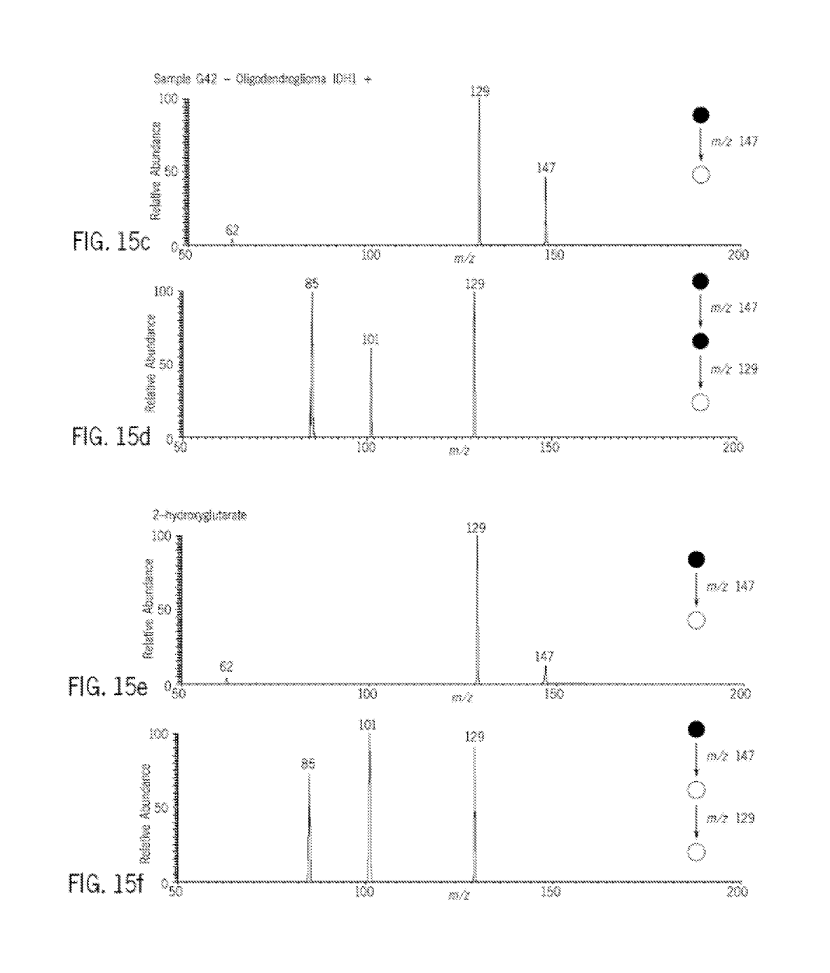

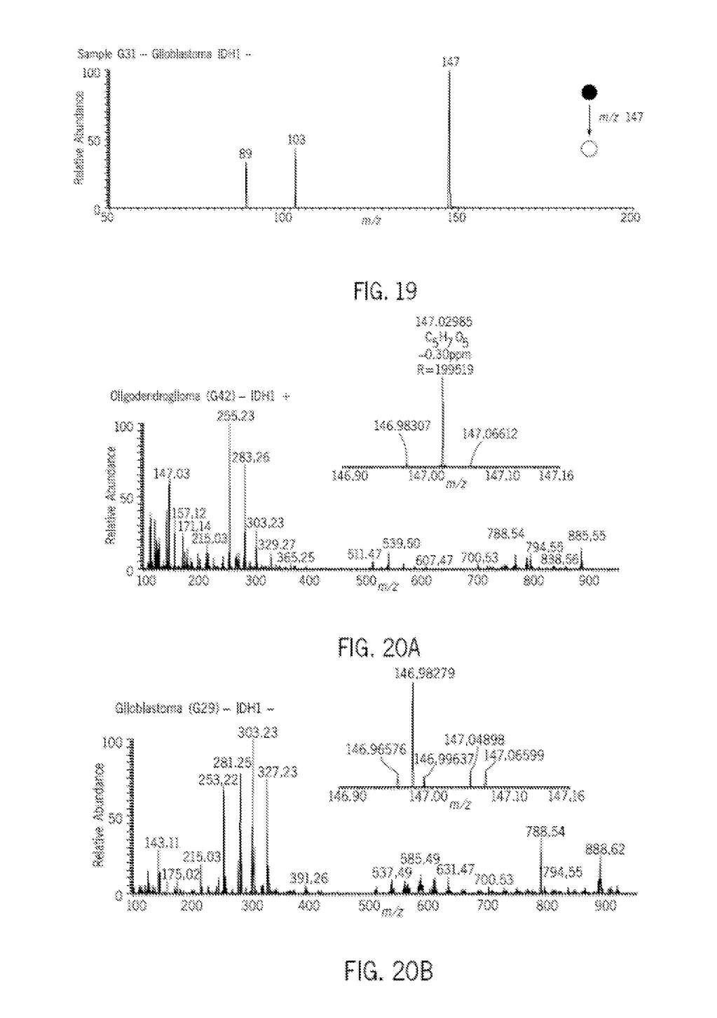

Tandem MS analysis (MS²) with a linear ion trap mass spectrometer was used to characterize the peaks at m/z 147. Tandem MS analysis of m/z 147 (the less intense peak noise levels) from a glioblastoma sample with wild-type IDH1 revealed main fragment ions at m/z 89 and m/z 103 (FIG. 19). However, in an oligodendroglioma with the IDH1 R132H mutation, the main fragment ion generated from m/z 147 was m/z 129, which corresponds to a neutral loss of a water molecule from 2-HG (FIG. 15c). Further characterization of m/z 129 with an additional round of tandem MS analysis (MS³) yielded two additional fragment ions at m/z 101 and m/z 85, corresponding to neutral losses of CO and CO₂, respectively (FIG. 15d). Identical MS² and MS³ results were obtained when purified L-α-hydroxyglutaric acid was subjected to tandem MS experiments (FIG. 15e,f).

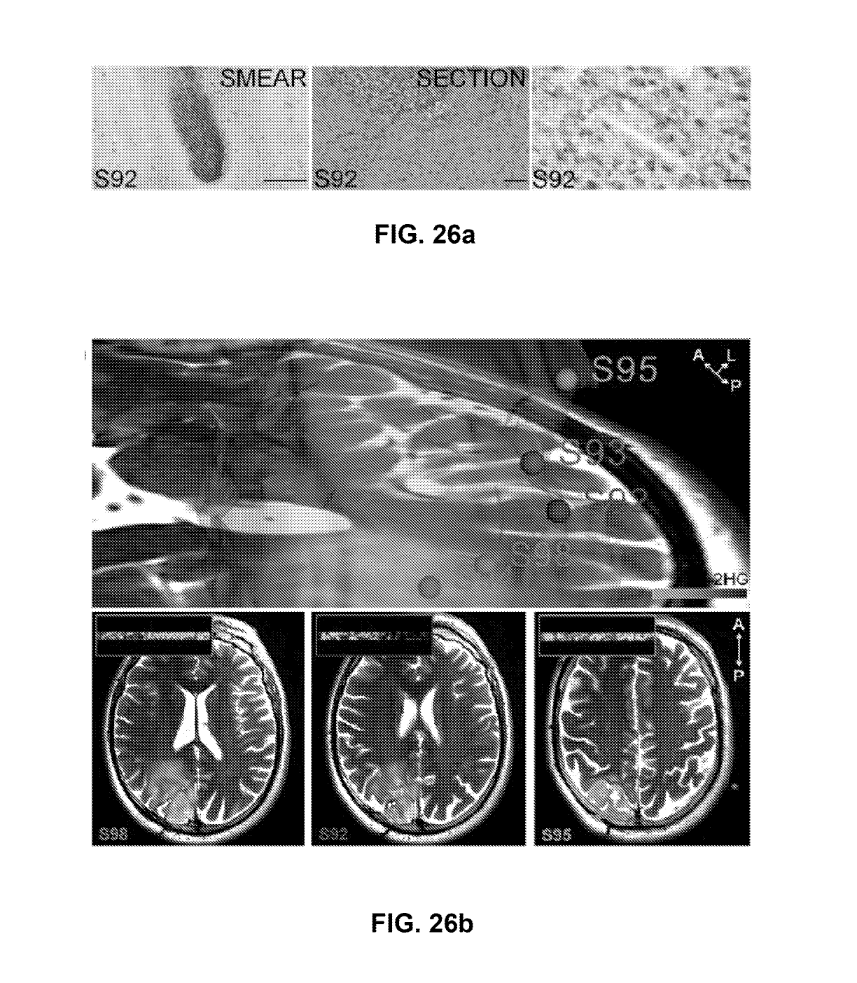

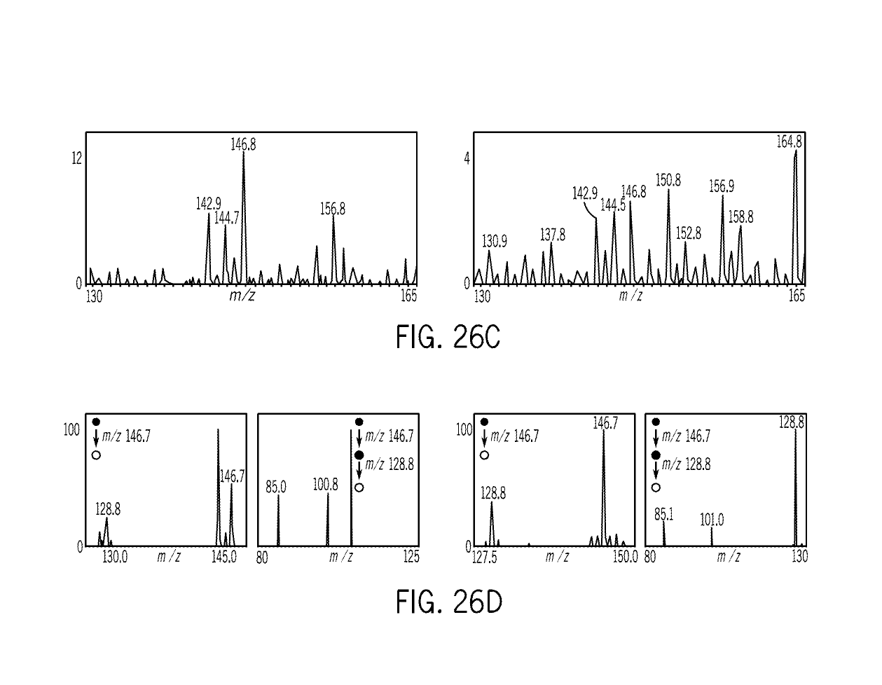

The peaks were further characterized using a high-resolution LTQ Orbitrap mass spectrometer. DESI-MS mass spectra from an IDH1 R132H mutant sample showed a prominent peak at m/z 147.02985, with a mass resolution of ˜200,000 in the negative ion mode (FIG. 26). This matches the 2-HG molecular formula (C₅H₇O₅) with a very low mass error of 0.3 ppm. Tandem MS of the standard 2-HG at m/z 147.02982 using high resolution MS confirmed the main fragment at m/z 129.01953 which corresponds to neutral loss of water (C₅H₅O₄, 1.7 ppm mass error), and MS³ fragments m/z 101.02455 (C₄H₅O₃, 1.32 ppm mass error) and m/z 85.02966 (C₄H₅O₂, 1.88 ppm mass error) that correspond to further neutral losses of CO and CO₂ from m/z 129, respectively (data not shown). In all, these results confirm the ability to reliably detect 2-HG with DESI-MS.

2-Hydroxyglutarate Levels by DESI-MS in Gliomas Correlates with Mutational Status

The levels of 2-HG were next monitored using DESI-MS in a panel of 35 human glioma resection specimens. These samples included primary and recurrent oligodendrogliomas, oligoastrocytomas and astrocytomas of different grades, including Grade IV glioblastoma samples (Table 6). The presence of the R132H mutation in IDH1 was determined by immunohistochemistry using a previously validated antibody that selective recognizes the R132H mutant epitope and not the wild-type epitope from IDH1 (Table 6). 2-HG levels were measured using a linear ion trap LTQ DESI-MS rapidly, directly from frozen tissue sections, and without any sample preparation. The 2-HG signal at m/z 147 was normalized to the levels of the forty most abundant lipid species detected from the glioma samples. This allowed the relative levels of 2-HG to be determined from each sample. Levels of 2-HG in R132 mutant IDH1 tumors ranged from 5 to 35 μmol per gram of tumor. Nearly all of the tumors that lacked the R132H mutation had over 100-fold less 2-HG (Table 6).

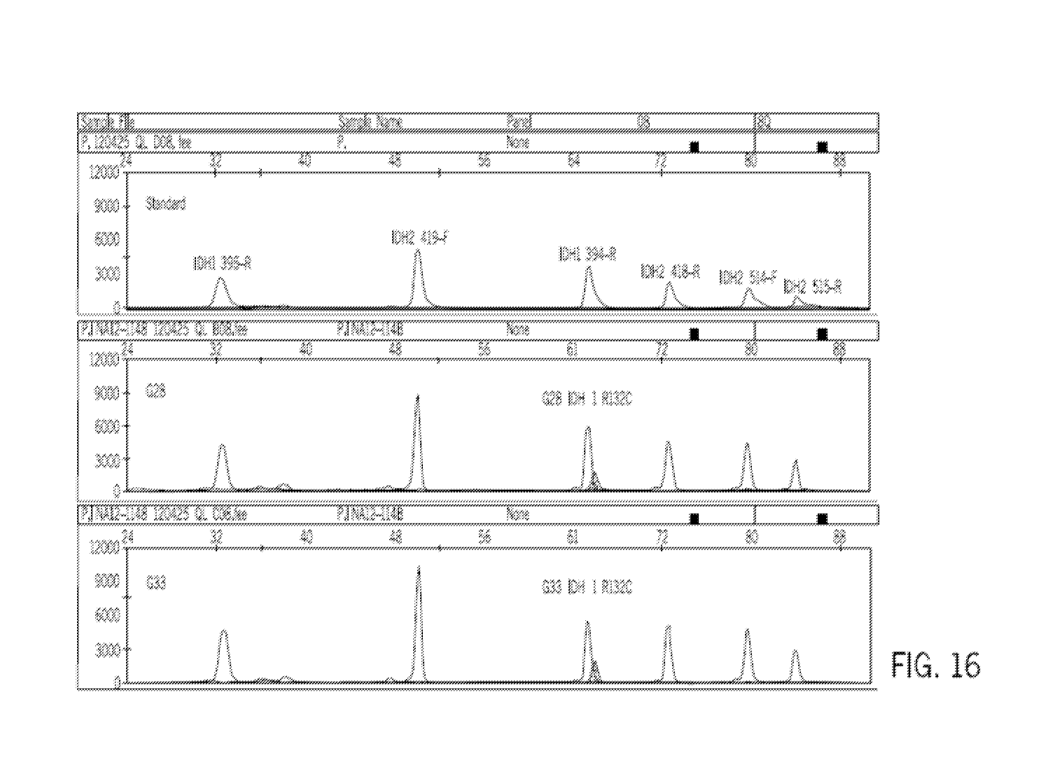

Interestingly, however, two samples (G28 and G33) that did not react with the R132H mutant-detecting antibody demonstrated a significant peak at m/z 147 (data not shown). The presence of 2-HG was confirmed by both tandem MS and high mass resolution experiments (data not shown). These findings suggested that the samples possessed alternate mutations in IDH1 or IDH2 that would generate the onco-metabolite, 2-HG. To address this possibility, targeting sequencing was performed for all of the major mutations in IDH1 and IDH2 that have been described in gliomas. This analysis revealed that both of these samples harbored a less common but previously described mutation in IDH1 that leads to amino acid change R132C (FIG. 16). While this mutant enzyme generates 2-HG, it is not recognized by the antibody that reacts with the IDH1 R132H mutant. These results provide a notable example of how detection of 2-HG with DESI-MS allows rapid and accurate determination of IDH1 status in human gliomas, independent of the underlying genetic mutation in IDH1 and with very high sensitivity and specificity.

Two-Dimensional DESI-MS Imaging of Glioma Sections

To further validate the ability to identify IDH1 mutant tumor tissue, a recently developed method for studying the spatial distribution of molecules across a tissue section was pursued, namely, two-dimensional (2D)-DESI-MS ion imaging. Because DESI-MS imaging does not destroy a sample as it is being analyzed, the same tissue section can be stained with H&E. Thus, a user can overlay the quantitative spatial information that 2D-DESI-MS provides onto the optical image of the tissue section. This facilitates correlating molecular signals such as 2-HG levels or tumor lipid levels with the underlying histopathology.

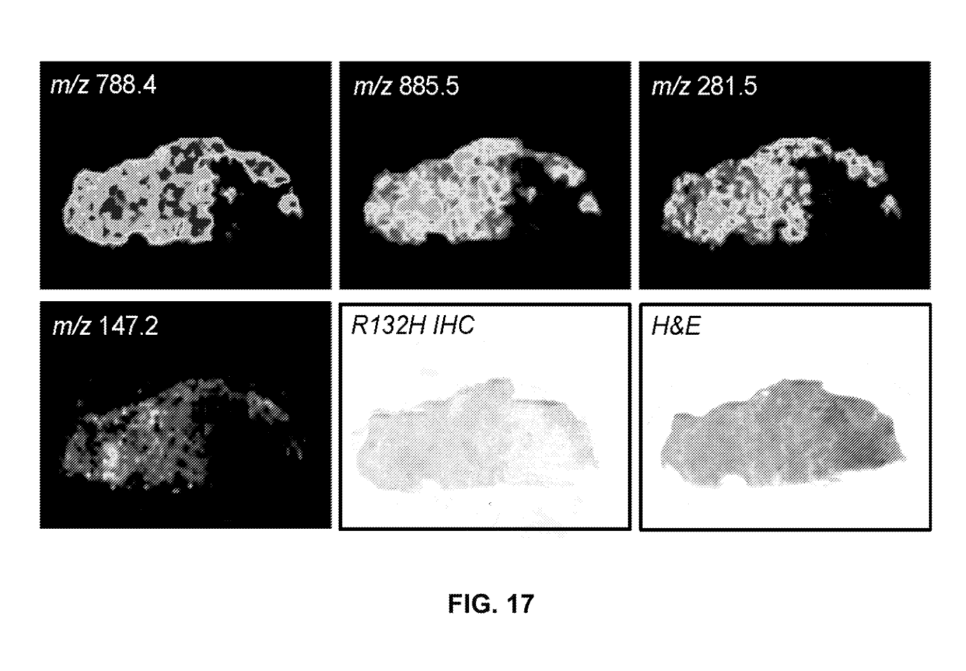

As an example, 2D-DESI-MS was used to evaluate the distribution of 2-HG and other diagnostic lipid species in a number of the glioma specimens which were previously characterized in Table 6. DESI-MS ion images of a densely cellular glioblastoma with wild-type IDH1, showed characteristic lipid species but m/z 147 was not detected (data not shown). In contrast, in a densely cellular glioblastoma with mutant IDH1, accumulation of 2-HG (m/z 147) was observed in the region with high tumor cell concentration and was essentially absent in an abutting region containing only hemorrhage. Lipid species that are characteristic for glioblastoma (m/z 788.4, m/z 885.5 and m/z 281.5) fully overlapped with the distribution of 2-HG (FIG. 17). Similar borders between IDH1 mutant tumor cells and regions of non-neoplastic brain tissue were observed in other samples (data not shown). These results provide a clear visual demonstration that DESI-MS can rapidly discriminate tumor cells with mutations in IDH1 from tissues without mutations in IDH1.

Tumor Margin Delineation for IDH1 Mutated Surgical Cases Using 2-HG Levels

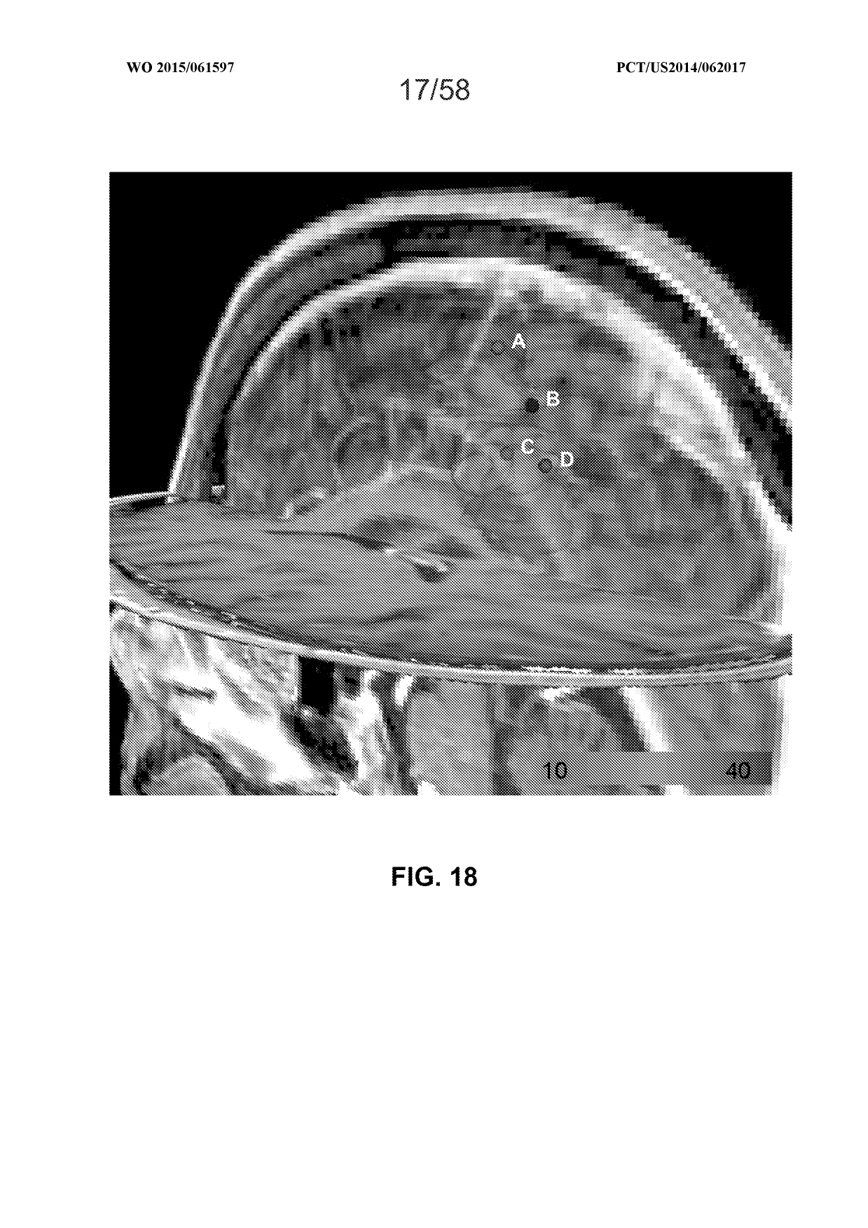

Above, with 2D-DESI-MS imaging, a visual demonstration that IDH1 mutant tumor tissues can be discriminated from normal tissues was provided. Intraoperatively, this ability could help detect glioma margins, i.e. where glioma cells interface with non-neoplastic brain tissue. Integrating the 2-HG information derived from DESI-MS with a patient's radiological imaging data would greatly empower a surgeon's intraoperative decision making To integrate these two forms of information, samples were collected from five neurosurgical resections of IDH1 mutant tumors performed with 3D mapping and registration in the Advanced Multimodality Image Guided Operating (AMIGO) Suite. This advanced surgical and interventional environment at Brigham and Women's Hospital is a part of the National Center for Image-Guided Therapy. The five cases included Grade II and III oligodendroglioma and oligoastrocytoma. The presence of the IDH1 R132H mutation was demonstrated in each case by immunohistochemistry. Tumor cell concentration was determined by a microscopic visual review of the H&E stained sections and of the IDH1 R132H immunostained sections. Strong 2-HG signals were identified in samples from the center of the tumor mass that were comprised of dense tumor (FIG. 18, example from case #3, Table 7). Biopsies from the margins of the radiographic mass contained low concentrations of infiltrating glioma cells as determined by H&E and IDH1 R132H stains. In those samples low to negligible levels of 2-HG were detected (FIG. 18, example from case #3). As the level of 2-HG indicates the tumor cell concentration in the total tumor volume, this methodology could be very valuable for detecting tumor margins during surgical interventions.

### Discussion:

In this report, it has been demonstrated that 2-hydroxyglutarate (2-HG) can be detected in glioma tissues using 2-dimensional DESI-MS. By monitoring 2-HG levels in tumor samples, at the time of surgery, this approach can provide rapid diagnostic information and actionable feedback.

Frozen tissue samples can be readily analyzed with the platform described herein. Fulfilling the true promise of this approach will, however, require the eventual development of surgical tools that allow DESI-analysis directly from tissue sampled by the neurosurgeon from the tumor resection bed. Nonetheless, with this current study a new paradigm for clinical diagnostics can be proposed. It has been previously demonstrated that many tumor types can be discriminated based on their lipid profile. Here, using gliomas with IDH1 mutations as an example, it has been shown that a single metabolite analyzed in the procedure room can rapidly provide highly relevant information: tumor classification (i.e. 2-HG expressing CNS tumors are nearly always gliomas), genotype information (i.e. 2-HG expressing tumors carry mutations in IDH1 or IDH2), prognostic information (i.e. 2-HG expressing tumors have a more favorable outcome) as well as intraoperative guidance for discriminating tumor from normal brain tissue. Presumably the approach described here should be applicable for the resection of all 2-HG producing tumors including chondrosarcoma and cholangiocarcinoma. Because ˜70-80% of grade II and grade III gliomas as well as the majority of secondary glioblastomas contain IDH mutations, monitoring 2-HG with DESI-MS could be useful for many neurosurgical interventions.

Other metabolites such as succinate and fumarate, which accumulate in specific tumor types, may similarly prove to be valuable markers using DESI-MS approaches. As metabolomic discovery efforts intensify, the cadre of useful metabolite markers and signatures is expected to expand significantly. This will undoubtedly increase the breadth and potential of MS diagnostics.

Two-dimensional DESI-MS analysis provides excellent spatial resolution without damaging the tissue, which can subsequently be stained with H&E dyes and visualized by standard light microscopy. Because the analyzed tissue remains intact, correlating the amount of metabolite with its originating source (i.e., stroma, blood vessel, tumor or normal non-neoplastic tissue) is now possible and practical. In addition, monitoring metabolite profiles simultaneously with lipid profiles (and their lipid-based tumor classifiers) as was done in this study will add to the diagnostic specificity and expand our understanding of tumor cell heterogeneity at a precise molecular level. Moreover, three-dimensional MRI mapping allows a correlation between radiologic imaging features and abundance of metabolites. Intraoperatively, in advanced multimodality image guided operating facilities, a surgeon could review visual information of the resection field and DESI-MS information about metabolite abundance and tumor classifiers all in the context of pre-operative and intra-operative radiological landmarks. Fluidly integrating all of this information, in a rapid timeframe, should significantly enhance a surgeon's capacity to achieve optimal tumor resection and would provide the foundation for surgery guided by metabolite-imaging mass spectrometry.

### Materials and Methods:

Tissue Samples

The tissue samples used in this study were obtained from the BWH Neurooncology Program Biorepository collection as previously described. They were obtained and analyzed under Institutional Review Board protocols approved at BWH. Informed written consent was obtained by neurosurgeons at BWH. The samples were sectioned for DESI-MS analysis as previously described. The tumors were classified in accordance with the WHO classification system. Resections of brain tumor lesions were performed using neuronavigation, with stereotactic mapping and spatial registering of biopsies performed as previously described. 3D-reconstruction of the tumor from MRI imaging data was achieved with 3-dimensional Slicer software package.

Histopathology and Immunohistochemistry

In addition to banked snap frozen samples, all cases had tissue samples that were formalin-fixed and paraffin embedded. Sections of FFPE tissue were stained with an anti-isocitrate dehydrogenase 1 (IDH1)-R132H antibody (clone HMab-1 from EMD Millipore) as previously described. Tissues were sectioned and immunostained as previously described. Hematoxylin and eosin (H&E) stained serial tissue sections were scanned using Mirax Micro 4SL telepathology system from Zeiss to generate digital optical images. Tumor content was evaluated by a trained neuropathologist (S. Santagata) through examination of H&E stained tissue sections and IDH1 R132H stained sections.

Identification and Quantification of 2-Hydroxyglutarate by DESI-MS

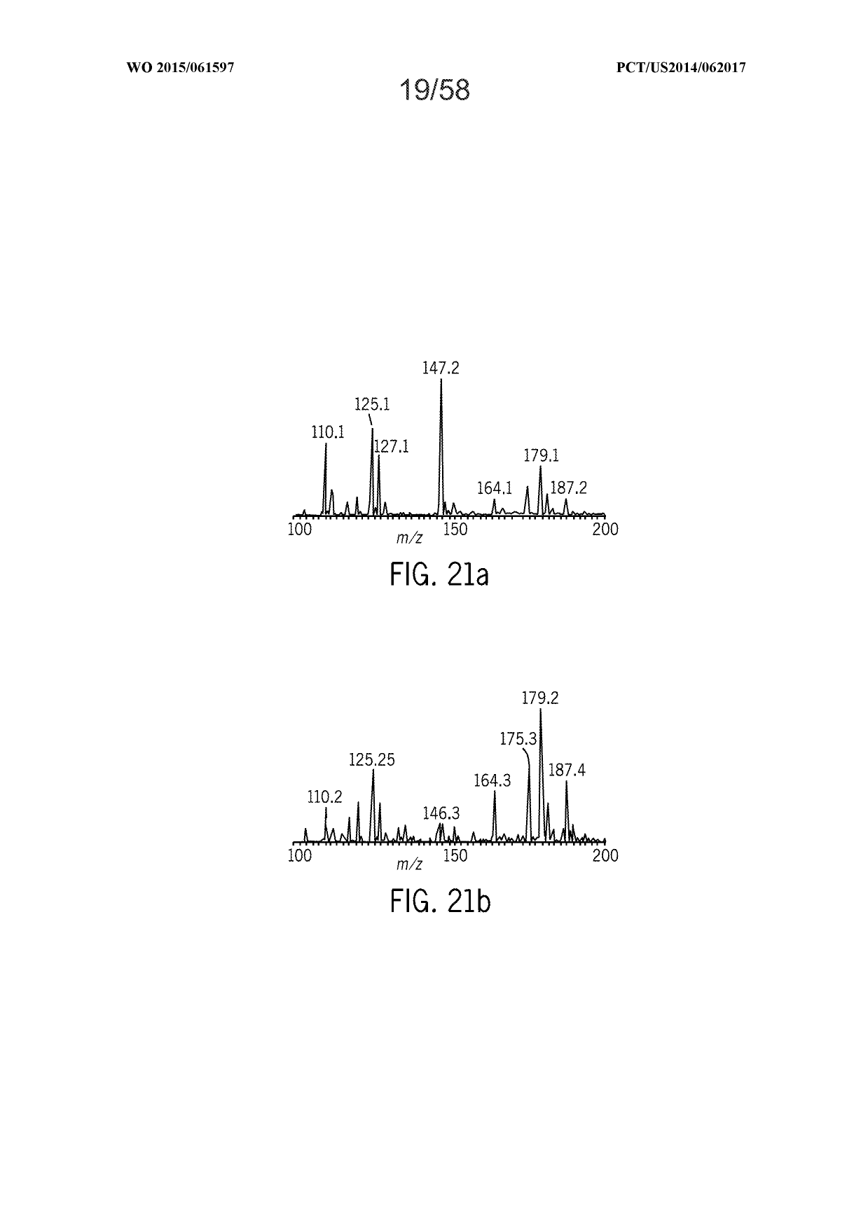

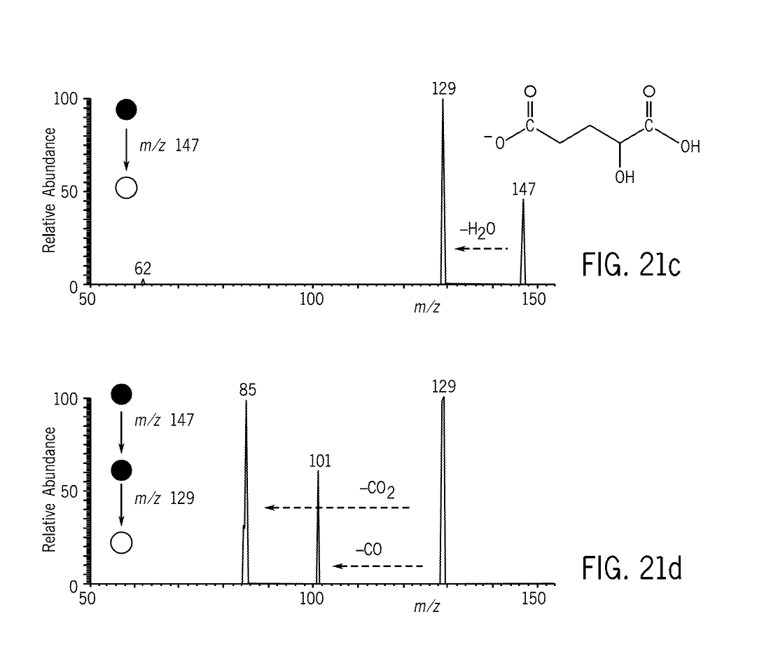

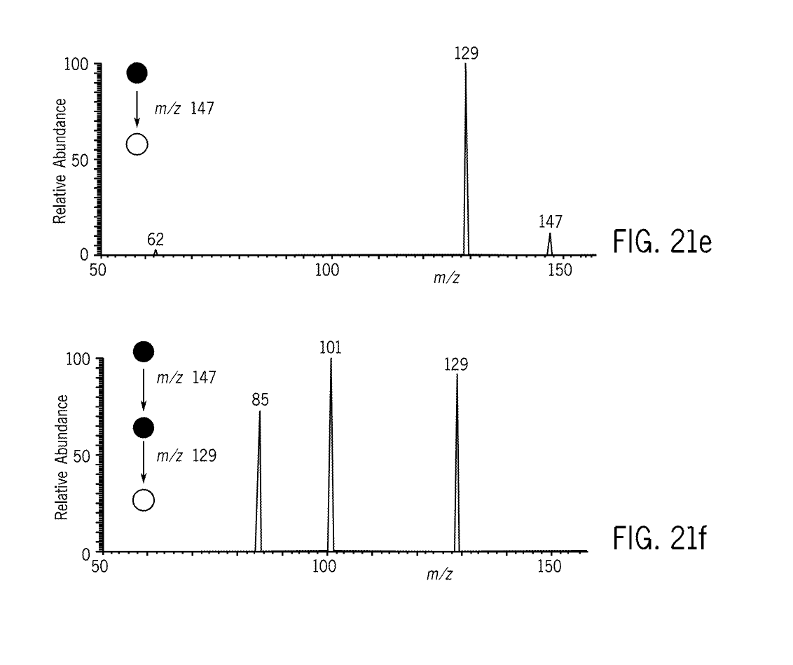

To determine if 2-HG could be detected directly from glioma tissue sections by DESI-MS, human glioma samples were tentatively analyzed in the negative ion mode using MeOH:H₂O (1:1) and ACN:DMF (1:1) as solvent systems from m/z 100-1100. A description of the samples used in this study is shown in Table 6. The IDH1 status of the specimens was initially evaluated by IHC of a piece of FFPE tissue. For stereotactic cases, all biopsies were less than 0.4 cm and these specimens were divided in two (one portion was frozen for DESI-MS studies and the other was processed for FFPE; the latter was used for IDH1 IHC). Experiments were initially performed using an LTQ linear ion trap mass spectrometer (Thermo Fisher Scientific, San Jose, Calif., USA). Negative ion mode DESI-MS mass spectra of samples G23, and G31 are shown in FIG. 21, using MeOH:H₂O (1:1) as the solvent system. Tandem MS analysis was used for identification of the molecules species at m/z 147.2 using the linear ion trap mass spectrometer. Further characterization was performed by MS³. The standard compound, L-α-Hydroxyglutaric acid disodium salt was purchased from Sigma-Aldrich Inc., Milwaukee, Wis. and was subjected to tandem MS experiments under the same conditions. Confirmation experiments were performed using a high-resolution LTQ Orbitrap mass spectrometer (Thermo Fisher Scientific, San Jose, Calif., USA). Thirty-five human gliomas samples were analyzed including oligodendrogliomas, astrocytomas, and oligoastrocytomas of different grades and varying tumor cell concentrations using a linear ion trap LTQ mass spectrometer. Note that as tissue analysis by DESI-MS is performed without sample preparation but directly on tissue section, standard quantification of 2-HG as commonly performed with time consuming HPLC-MS protocols is not possible. One means by which relative levels of a certain molecule can be calculated is by normalizing its signal to a reference signal or set of signals obtained from the sample. In this study, the total abundance of 2-HG signal at m/z 147 was normalized to the sum of total abundance of the forty most abundant lipid species detected from the glioma samples by DESI-MS. As a small contribution of background signal at the same m/z 147 was present in DESI mass spectra, MS² was performed for all samples in order to confirm the presence of 2-HG. This was especially important in some IDH1 mutant samples with low tumor cell concentrations and therefore much lower abundances of 2-HG in DESI mass spectrum. If the MS² and MS³ fragmentation pattern matched that of authentic 2-HG, the sample was determined to be IDH1 mutated. Discrepancies in the fragmentation pattern or absence of detectable levels of m/z 147 were interpreted as IDH wild-type by MS analysis. Results for DESI-MS analysis were obtained using two solvent systems. Note that while the solvent system DMF:ACN (1:1) favored relative abundances of low m/z ions when compared to MeOH:H₂O, similar trends in 2-HG were observed for both solvents. Interestingly, the ratio of m/z 147 to the sum of lipid species correlated with the tumor cell concentration determined for the sample by histopathological evaluation of serial tissue section, providing a direct measure of the 2-HG levels in tissue. Most samples that were negative for IDH1 mutation as determined by IHC did not present 2-HG in the DESI-MS mass spectra, even if the sample presented high tumor cell concentration, as confirmed by tandem MS analysis (with the exception of two samples as noted in the text).

In DESI-MS analysis, a tissue section of ˜12 μm in thickness is examined in a pixel by pixel fashion, with a sampling area of 200×200 μm² for each mass spectra acquired. A rough estimation of the total amount of 2-HG/pixel can be made by first estimating the mass of a 10 mm×6 mm human brain tissue section of 12 μm thickness to be ˜0.5 mg. Each 200×200 μm² pixel therefore contains a mass of 3.3×10⁻⁴ mg. From literature values, it can be then estimated that each pixel being sampled by DESI-MS spray in R132 mutant IDH1 tumors has between 2 and 12 pmol of 2-HG. Therefore, it is expected that the concentration of 2-HG/pixel in wild-type IDH1 tumors would not be within the detectable levels for DESI-MS analysis. To address this, the limit of detection of 2-HG was estimated by depositing different concentrations of standard 2-HG solutions onto mouse brain tissue, followed by DESI-MS analysis under the same experimental conditions that human glioma samples were analyzed. As observed, while a linear relationship between 2-HG concentration and total abundance of m/z 147 was not observed (R²=0.69), a somewhat linear relationship was achieved between 2-HG concentration and total abundance of m/z 147 normalized to the sum of total abundance of the forty most abundant lipid species detected (R²=0.94) from the mouse brain tissue by DESI-MS. These results indicate that the value of m/z 147 abundance normalized to the lipid signals provides an indication of the concentration of 2-HG in tissues. The limit of detection was roughly estimated to be approximately 3 μmol 2-HG/gram of tissue, which is lower than the reported levels of 2-HG in R132 mutant IDH1 tumors.

One of the challenges in the analysis was to determine IDH1 status by DESI-MS detection of 2-HG in samples with low tumor cell concentration from full mass spectral data. For these samples, low detectable values of m/z 147 could be initially assumed as an indication of IDH negative mutation. Nevertheless, MS² and MS³ of m/z 147 enabled IDH+ status confirmation for these samples, despite the low tumor cell concentration. DESI-MS imaging was performed for a few of the samples analyzed to evaluate the distribution of 2-HG and other diagnostic lipid species compared to tumor cell distribution in tissue

Section IV

For many intraoperative decisions, surgeons depend on frozen section pathology, a technique developed over 150 years ago. Technical innovations that permit rapid molecular characterization of tissue samples at the time of surgery are needed and in most cases, during the surgical procedure. Here, using desorption electrospray ionization mass spectrometry (DESI MS), the tumor metabolite 2-hydroxyglutarate (2-HG) was rapidly detected from tissue sections of surgically-resected gliomas, under ambient conditions and without complex or time consuming preparation. With DESI MS, IDH1-mutant tumors were identified with both high sensitivity and specificity within minutes, immediately providing critical diagnostic, prognostic and predictive information. Imaging tissue sections with DESI MS shows that the 2-HG signal overlaps with areas of tumor and that 2-HG levels correlate with tumor content, thereby indicating tumor margins. Mapping the 2-HG signal onto three-dimensional MRI reconstructions of tumors allows the integration of molecular and radiologic information for enhanced clinical decision-making. The methodology and its deployment in the operating room were also validated—a mass spectrometer has been installed in an Advanced Multimodality Image Guided Operating (AMIGO) suite and the molecular analysis of surgical tissue during brain surgery has been demonstrated. This work indicates that metabolite-imaging mass spectrometry could transform many aspects of surgical care.

Introduction:

The review of tissue sections by light microscopy remains a cornerstone of tumor diagnostics. In recent decades, monitoring expression of individual proteins using immunohistochemistry and characterizing chromosomal aberrations, point mutations and gene expression with genetic tools has further enhanced diagnostic capabilities and this capability is demonstrated here. These ancillary tests, however, often require days to weeks to perform and the results become available long after surgery is completed. For this reason, the microscopic review of tissue biopsies frequently remains the sole source of intraoperative diagnostic information, with many important surgical decisions such as the extent of tumor resection based on this information. This approach is time consuming, requiring nearly 30 minutes between the moment a tissue is biopsied and the time the pathologist's interpretation is communicated back to the surgeon. Even after the report of the final pathologic diagnosis is issued days later, a lot of diagnostic, prognostic and predictive information is left undiscovered and unexamined within the tissue. Tools that provide more immediate feedback to the surgeon and the pathologist and that also rapidly extract detailed molecular information could transform the management of care for cancer patients.

Mass spectrometry offers the possibility for the in-depth analysis of the proteins and lipids that comprise tissues. It has been shown that desorption electrospray ionization mass spectrometry (DESI MS) is a powerful methodology for characterizing lipids within tumor specimens. The intensity profile of lipids ionized from within tumors can be used for classifying tumors and for providing valuable prognostic information such as tumor subtype and grade. Because DESI MS is performed in ambient conditions with minimal pretreatment of the samples, there is the potential to provide diagnostic information rapidly within the procedure room. The ability to quickly acquire such valuable diagnostic information from lipids prompted us to determine whether DESI MS could be used to detect additional molecules of diagnostic value within tumors such as their metabolites.

Recently, recurrent mutations have been described in the genes encoding isocitrate dehydrogenases 1 and 2 (IDH1 and IDH2) in a number of tumor types including gliomas, intrahepatic cholangiocarcinomas, acute myelogenous leukemias (AML) and chondrosarcomas 15. These mutant enzymes have the novel property of converting α-ketoglutarate to 2-hydroxyglutarate (2-HG). This oncometabolite has pleiotropic effects on DNA methylation patterns, on the activity of prolyl hydroxylase and on cellular differentiation and growth. While 2-HG is present in vanishingly small amounts in normal tissues, concentrations are extremely high in tumors with mutations in IDH1 and IDH2—several micromoles per gram of tumor have been reported in tumors. Several groups have reported that 2-HG can be detected by magnetic resonance spectroscopy and imaging hence providing a non-invasive imaging approach for evaluating patients. While such imaging approaches may provide information to plan surgery and to follow the response to chemotherapeutics, applying them to guide decision-making during an operation is currently impractical.

The ability to detect 2-HG intraoperatively would be particularly useful because infiltrating gliomas such as IDH1 mutant gliomas are difficult to visualize with conventional means which contributes to the high prevalence of suboptimal surgical resection. The more residual tumor is left, the shorter the patient survival for both low and high grade gliomas. Detecting infiltrating glioma cells by microscopic review is challenging on well-prepared H&E stained permanent sections, and even more so on H&E stained frozen sections which frequently harbor processing artifacts. Thus, 2-HG detection could help to define surgical margins thereby allowing for more complete resection and for longer survival. Moreover, directing patients toward appropriate clinical trials for targeted therapeutics would be facilitated by more rapid molecular categorization of tumors.

Here, it is shown that 2-HG can be rapidly detected from glioma samples using DESI MS—under ambient conditions, without complex tissue preparation and during surgery allowing rapid molecular characterization and providing information that is unattainable by standard histopathology techniques. The first implementation of mass spectrometry within an operating room for the molecular characterization of tissue as part of an image-guided therapy program is also presented. The findings were cross-validated using standard pathology techniques. Measuring specific metabolites in tumor tissues with precise spatial distribution and under ambient conditions provides a new paradigm for intraoperative surgical decision-making, rapid diagnosis, and patient care management.

Results:

Identification of 2-Hydroxyglutarate with DESI MS:

Referring to FIG. 21, negative ion mode DESI mass spectra are shown which were obtained using a linear ion trap mass spectrometer from m/z 100 to 200 for samples G23, an oligodendroglioma with the IDH1 R132H mutant (a), and G31, a glioblastoma with wild-type IDH1 (b). Tandem mass spectra of m/z 147 detected from sample G42, an oligodendroglioma with the IDH1 R132H mutant (MS2, (c); MS3, (d)) and from a 2-HG standard (MS2, (e); MS3, (f)).

To determine the conditions for detecting 2-hydroxyglutarate (2-HG) from glioma frozen tissue sections by DESI MS, the negative ion mode mass spectra were first recorded from two glioma samples: an oligodendroglioma with mutated IDH1 (encoding the amino acid change R132H) and a glioblastoma with wild-type IDH1. 2-HG is a small organic acid containing two carboxylic acid functional groups in its structure. In the negative ion mode, the deprotonated form of 2-HG should be detected at an m/z of 147.03 (C5H7O5-). Together with the rich diagnostic lipid information commonly observed from gliomas by DESI MS in the mass range m/z 100-1000, a significant peak was detected at m/z 147 in an IDH1 mutated sample (FIG. 21a), but not in an IDH1 wild-type sample (FIG. 21b).

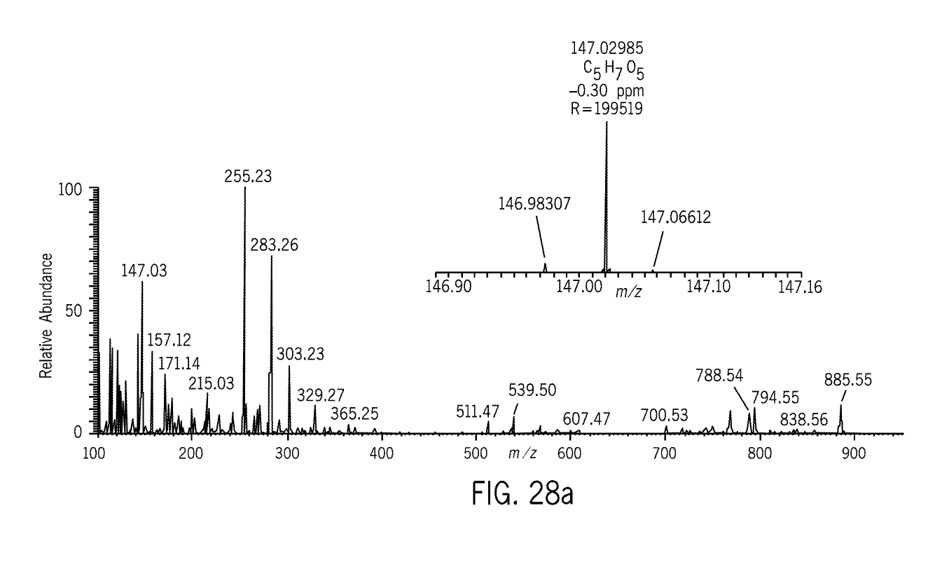

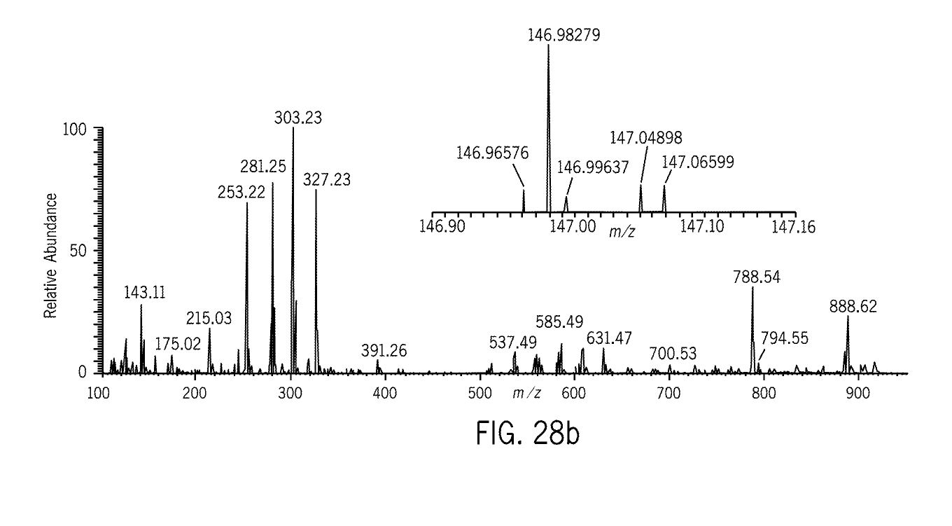

Tandem MS analysis (MS²) with a linear ion trap mass spectrometer was used to characterize the signal at m/z 147 (FIG. 21c-f). In an oligodendroglioma with the IDH1 R132H mutation, the main fragment ion generated from m/z 147 was m/z 129, which corresponds to loss of a water molecule from 2-HG (FIG. 21c). Further characterization of m/z 129 with an additional round of MS analysis (MS³) yielded two additional fragment ions at m/z 101 and m/z 85, corresponding to neutral losses of CO and CO₂, respectively (FIG. 21d). Identical MS2 and MS3 fragmentation patterns were obtained when purified L-α-hydroxyglutaric acid was subjected to tandem MS experiments (FIG. 21e,f). The peaks were further characterized using a high-resolution and high-mass accuracy LTQ Orbitrap mass spectrometer (FIG. 28). DESI mass spectra from an IDH1 R132H mutant sample showed a prominent peak at m/z 147.0299 in the negative ion mode, which matched the molecular formula of the deprotonated form of 2-HG (C₅H₇O₅⁻) with a mass accuracy of 0.3 ppm. In all, these results confirm the ability to reliably and rapidly detect 2-HG from human glioma tissue sections with DESI MS.

2-HG Levels Correlate with Mutational Status and Tumor Cell Content:

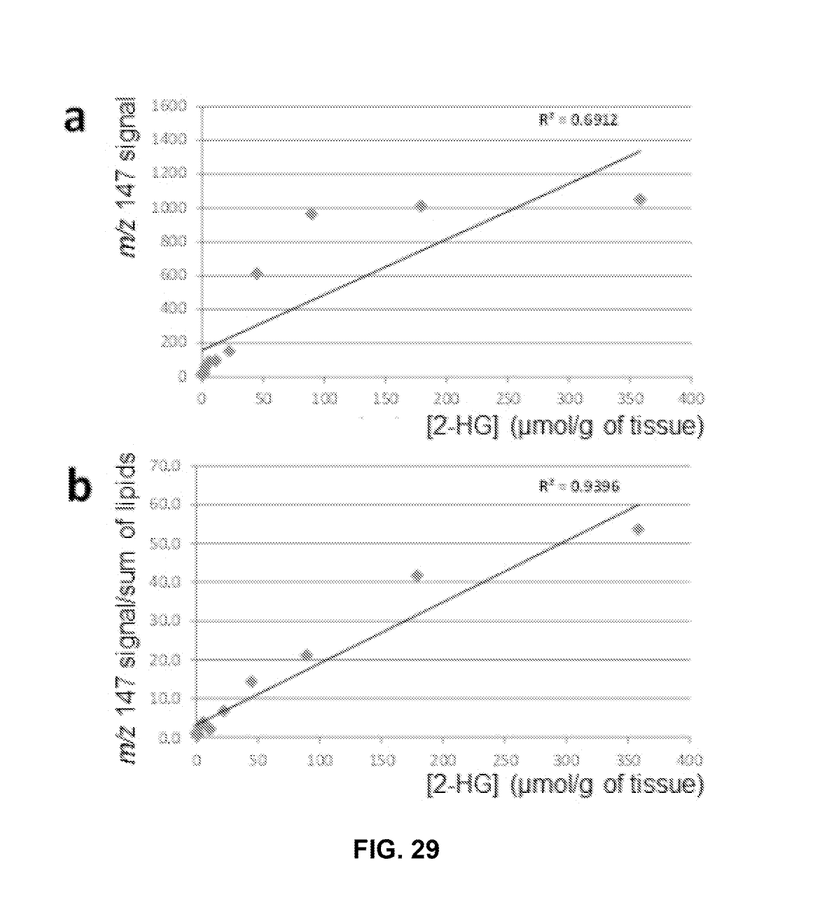

The levels of 2-HG were next monitored using DESI MS in a panel of 35 human glioma specimens (Table 8) including primary and recurrent oligodendrogliomas, oligoastrocytomas and astrocytomas of different grades. The samples were first characterized using a clinically validated antibody that selectively recognizes the R132H mutant epitope and not the wild-type epitope from IDH1 (Table 8). 21 of the 35 samples had the R132H mutation. 2-HG levels in these samples were then measured directly from frozen tissue sections using a linear ion trap LTQ DESI. In some samples, a peak at m/z 147 was detected and assigned to 2-HG by tandem MS (MS²) analysis, thereby providing strong independent evidence that these samples were mutated for one of the IDH genes. To account for the variability in desorption and ionization efficiency throughout the tissue and between samples, 2-HG signal was normalized to the combined intensity of the forty most abundant lipid species that were detected during each data acquisition (see, Table 8 and materials and methods for more details on normalization). In all of the 21 samples with the IDH1 R132H mutation, 2-HG was clearly detected with a limit of detection estimated to be on the order of 3 μmol 2-HG/g of tissue (FIG. 29), which is below the lowest concentration of 2-HG in tissue in IDH1 mutant human gliomas as measured by HPLC-MS analysis. In DESI-MS analysis, a tissue section of ˜12 μm in thickness is examined on a pixel by pixel basis, with sampling area of 200×200 μm2 for each mass spectra acquired. An estimation of the total amount of 2-HG/pixel can be made by first estimating the mass of a 10 mm×6 mm human brain tissue section of 12 μm thickness to be ˜0.5 mg. The limit of detection of 2-HG was estimated by depositing different concentrations of standard 2-HG solutions onto mouse brain tissue, followed by DESI MS analysis under the same experimental conditions as for the human glioma samples analysis. Referring to FIG. 29a, a correlation factor of (R²=0.69) was determined when directly plotting the m/z 147 signal versus the known 2-HG concentration, but referring to FIG. 29b, a somewhat linear relationship was observed between the m/z 147 normalized to the sum of the forty most abundant lipid species and the known 2-HG concentration (R²=0.94) from the mouse brain tissue by DESI MSI. Note that the correlation in both plots is significantly improved if the concentration range is limited to 100 μmol/g. The limit of detection was estimated to be 3 μmol 2-HG/gram of tissue.

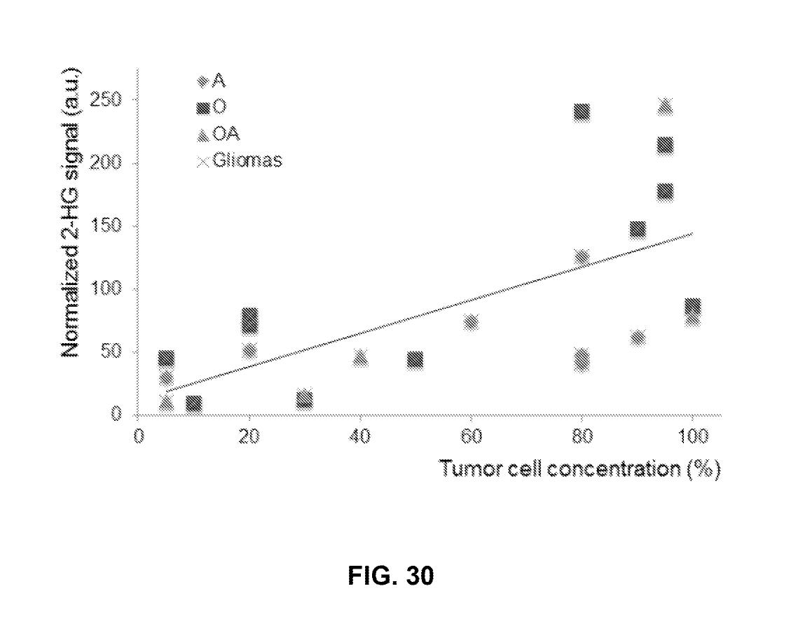

A correlation (R²=0.42) was also observed between the concentration of tumor cells and the intensity of the 2-HG signal—samples with low concentrations of tumor cells (<50%) had lower 2-HG levels while samples with high concentrations of tumor cells (>50%) had higher 2-HG levels (FIG. 30). All gliomas are represented by an X, oligodendrogliomas are represented by a square (O), oligoastrocytomas by a triangle (OA), and astrocytomas by a diamond (A). The astrocytoma series is comprised of all glioblastoma except for one astrocytoma grade II (G2) with 60% tumor cell concentration. The correlation factor for the trendline (R²) is 0.42. Although the sample set of high density tumors (≧80% tumor cells) is relatively small, it was noted that GBMs with mutant IDH1 generally had lower levels of 2-HG than oligodendrogliomas (FIG. 30).

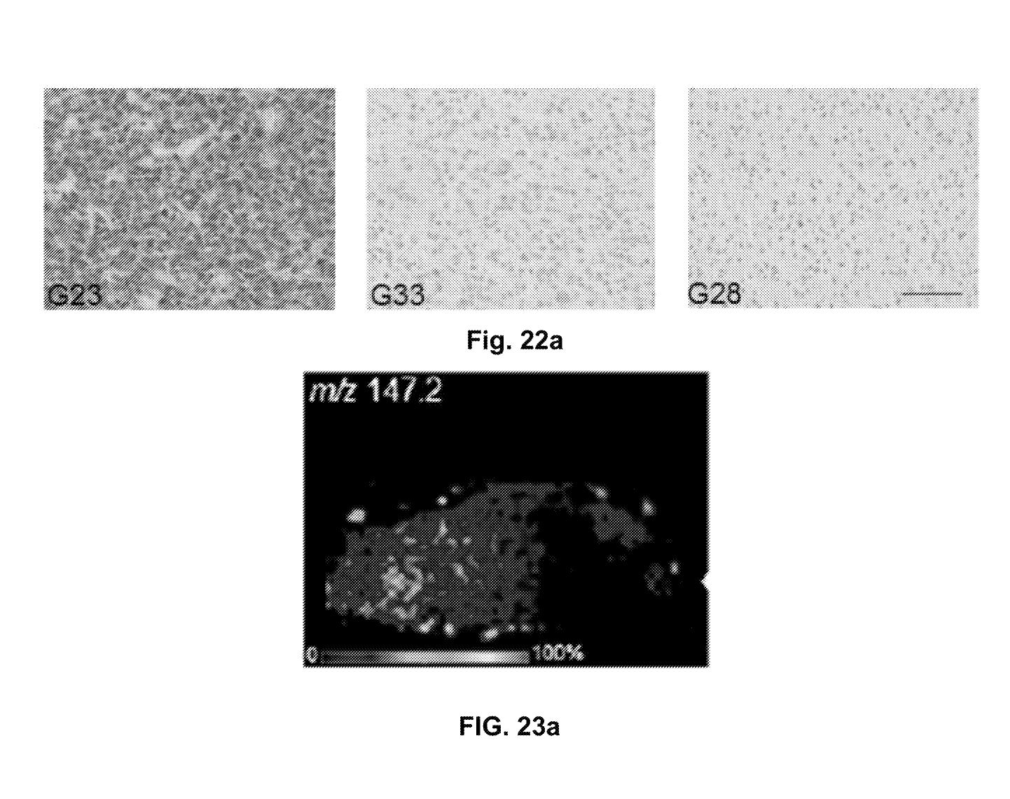

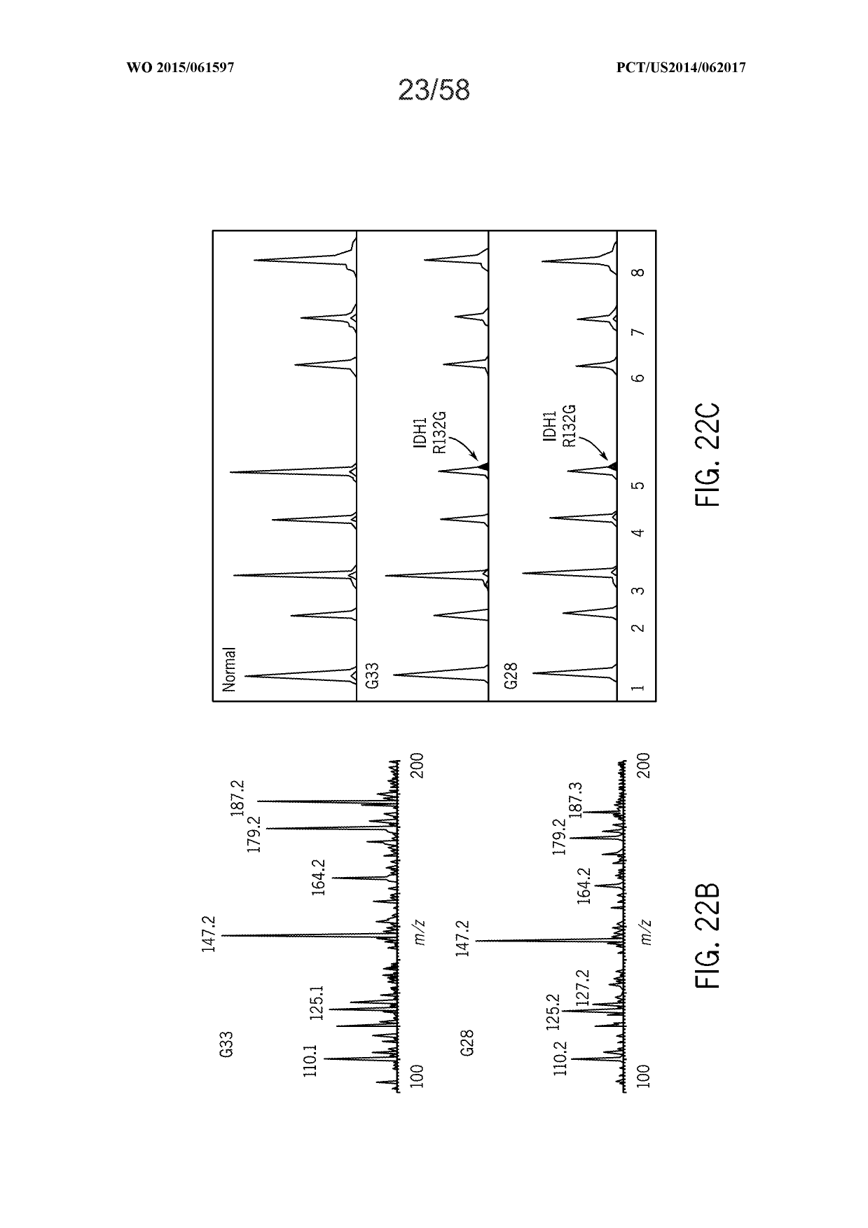

Interestingly, in two of the samples (G33 and G28) that were negative for the IDH1 R132H mutation by immunohistochemical staining (FIG. 22a), 2-HG signal was detected (FIG. 22b). The signal from both samples was confirmed to be from 2-HG by tandem MS analysis (MS2). Because other mutations in IDH1 or IDH2 can lead to 2-HG accumulation, targeted sequencing was performed for all of the major mutations in IDH1 and IDH2 that have been described in gliomas. This analysis revealed that both samples G33 and G28 harbored a less common but previously described IDH1 mutation that leads to substitution of amino acid arginine with glycine at position 132 (R132G) (FIG. 22c). These results provide a clear example of how detecting 2-HG with DESI MS allows rapid and accurate determination of IDH1 status in human gliomas. While the diagnostic antibody only recognizes one of the many IDH1 mutants, DESI MS captures the presence of 2-HG independent of the underlying genetic mutation in IDH1. Notably, the results show that DESI MS can detect 2-HG with very high sensitivity and specificity—2-HG signal was detected in all cases with mutant IDH1 (even when the tumor concentration was as low as 5%) and 2-HG signal was not detected in any of the cases with wild type IDH1.

Referring to FIG. 22a immunohistochemistry is shown using an IDH1 R132H point mutation specific antibody on formalin-fixed and paraffin embedded (FFPE) sections from glioma samples (G33 and G28), (scale bar, 100 μm). Referring to FIG. 22b, negative ion mode DESI mass spectra are shown which were obtained using a linear ion trap mass spectrometer for samples G33 and G28 that are negative for IDH1 R132H mutant immunohistochemistry. Referring to FIG. 22c, targeted mutational profiling was performed using SNaPshot analysis on nucleic acids extracted from GBM archival specimens (G33 and G28) run in parallel with a normal genomic DNA control, as indicated. The arrows point to the IDH1 R132G (c.394C>G) mutant allele identified in both tumor samples. The assayed loci were as follows: (1) KRAS 35; (2) EGFR 2236_50del R; (3) PTEN 517; (4) TP53 733; (5) IDH1 394; (6) PIK3CA 3139; (7) NOTCH1 4724 and (8) NOTCH1 4802.

2D DESI MS Imaging of 2-HG in Glioma Sections Delineates Tumor Margins:

To further validate DESI MS as a tool for monitoring 2-HG levels, two-dimensional (2D) DESI MS imaging was used to study the spatial distribution of molecules across a tissue section. DESI MS imaging has recently been shown not to destroy a sample as it is being analyzed when an histologically compatible solvent system is used. This relative preservation allows the same tissue section to be stained with H&E following DESI MS data acquisition and the spatial molecular information derived from DESI MS can then be overlaid onto the optical image of the tissue. As such, this approach provides a powerful way to correlate 2-HG levels with histopathology and, importantly, to validate the DESI MS observations.

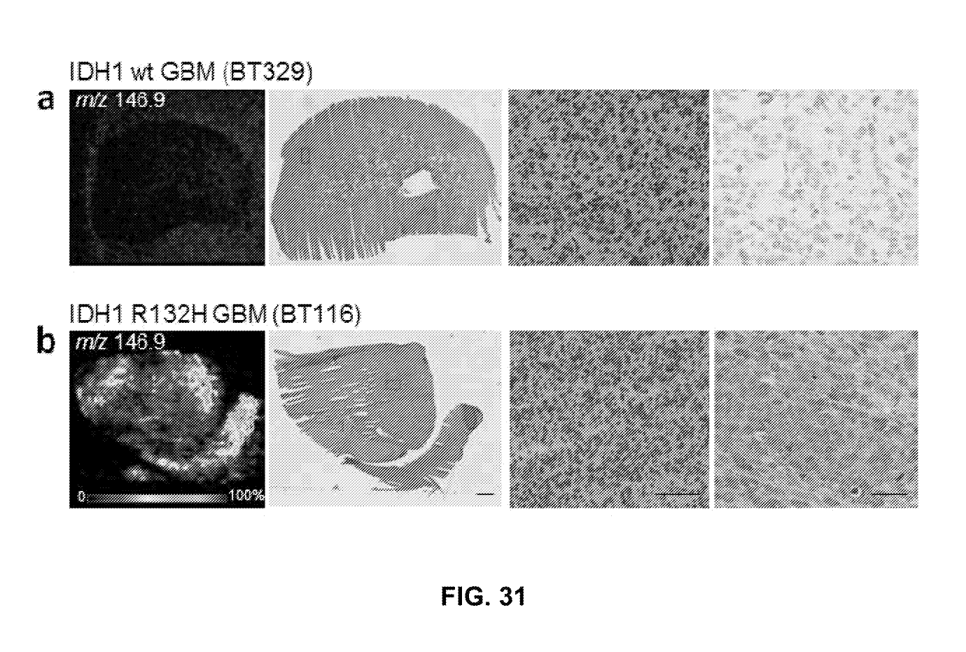

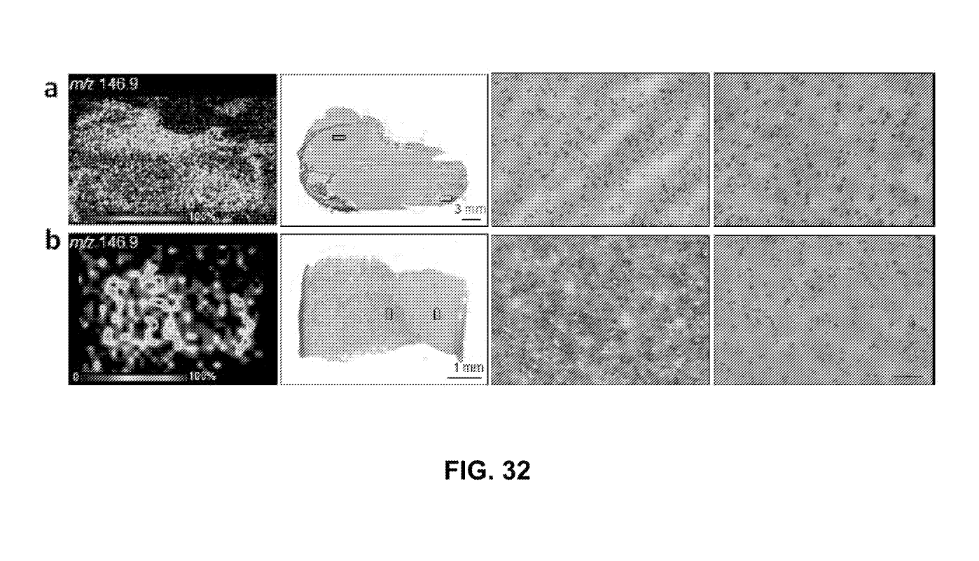

As a control, 2D DESI MS data was acquired from frozen sections of human glioblastoma orthotopic xenograft models that had been implanted into the brains of immunocompromised mice (FIG. 31). FIG. 31a shows negative ion mode two dimensional DESI MS images of human glioblastoma xenograft (BT329) that has wild-type IDH1. FIG. 31b shows negative ion mode two-dimensional DESI-MS images of human glioblastoma xenograft (BT116) that has an IDH1 R132H mutation. The left panel is an ion map demonstrating the relative signal intensity of peak m/z 146.9, which was confirmed to be 2-HG by tandem MS analysis (MS2 and MS3). Relative signal intensity (0-100%) is plotted for each specimen using a grey scale. Low magnification and high magnification light microscopy images of H&E stained sections are shown, and IDH1 R132H point mutation specific antibody staining is shown on the far-right panel (scale bar, 100 μm). A signal for 2-HG was not detected from xenografts of a glioblastoma cell line (BT329) that has wild-type IDH1 (FIG. 31a). Strikingly, however, a strong signal for 2-HG was found throughout the tissue section of the mouse brain that was diffusely infiltrated by a glioblastoma xenograft (BT116) that has the IDH1 R132H mutation (FIG. 31b), as was similarly observed in an IDH1 R132H mutated oligodendroglioma xenograft model by liquid extraction surface analysis (LESA) nano ESI-MS imaging.

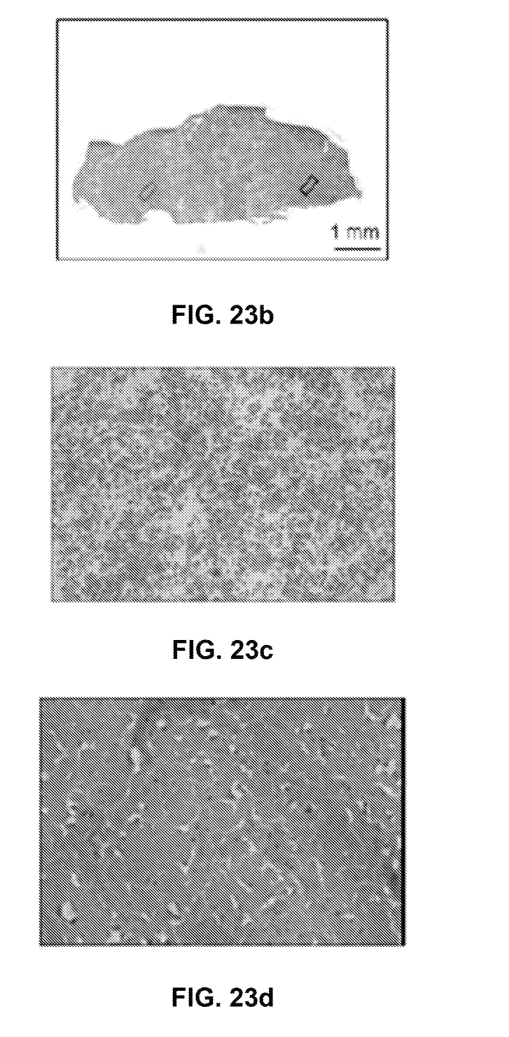

Tissue sections of human glioma specimens that had been surgically resected were next studied. Using 2D DESI MS with both an LTQ Ion Trap (Thermo Fisher Scientific, San Jose, Calif., USA) and an amaZon Speed ion trap (Bruker Daltonics, Billerica, Mass., USA), accumulation of 2-HG within a densely cellular glioblastoma with mutated IDH1 was observed (FIG. 23a-d). Negative ion mode two-dimensional DESI MS images from glioma resection specimens with IDH1 mutations. G30 (A-IV-O). FIG. 23a is an ion map demonstrating the relative signal intensity of peaks at m/z 146.7-147.2, which were each confirmed to be 2-HG by tandem MS analysis (MS²). Mass spectrometry data for sample G30 was acquired using an LTQ Ion Trap (Thermo Fisher Scientific, San Jose, Calif., USA). Relative signal intensity (0-100%) is plotted for each specimen using a grey scale. Low magnification light microscopy images of H&E stained sections show the tissue outline (FIG. 23b). The grey boxed area (the box on the left) indicates a region of higher tumor cell concentration. FIG. 23c is a magnified image of the tissue located at or near this box. Black boxed area (the box on the right) indicates blood. FIG. 23d is a magnified image of the tissue located at or near this box. Scale bars as indicated or 100 μm in FIGS. 23c and 23d. 2-HG was absent in an area of hemorrhage abutting the tumor (FIG. 23a).

In tissue specimens from two additional glioma resections, areas that contained regions of tumor were identified as well as regions of brain with only scattered infiltrating glioma cells—i.e. within the margin on the tumor. Referring to FIG. 32, negative ion mode two-dimensional DESI MS images were acquired from glioma resection specimens with IDH1 mutations. FIG. 32a shows S55 (OA-II) and FIG. 32b shows D31 (A-III). The left panel is an ion map demonstrating the relative signal intensity of peaks at m/z 146.7-147.2, which were each confirmed to be 2-HG by tandem MS analysis (MS2). Mass spectrometry data were acquired using an amaZon Speed ion trap (Bruker Daltonics, Billerica, Mass., USA). Relative signal intensity (0-100%) is plotted for each specimen using a grey scale. Low magnification light microscopy images of H&E stained sections show the tissue outline. The boxed area in the lower right of the second column of FIG. 32a and the boxed area in the middle of the image in the second column of FIG. 32b indicate regions of higher tumor cell concentration. The third column of FIGS. 32a and 32b are magnified images of tissues located within these boxes. The boxed area in the upper left of the image in the second column of FIG. 32a and the boxed area in the right of the image in the second column of FIG. 32b indicate rare infiltrating tumor cells. The right-most column of FIGS. 32a and 32b are high magnification images of tissues located within these boxes. Scale bars as indicated or 100 μm in the panels in the two right-most columns. DESI MS revealed strong 2-HG signals in the cellular portions of these samples but weaker signals in the portions of brain with scattered infiltrating tumor cells (FIG. 32a,b). By validating the DESI MS results directly with tissue histopathology, it was shown that monitoring 2-HG levels with DESI MS can help to readily discriminate tissue with dense tumor from tissue with only scattered tumor cells. Such discriminatory capacity can help define tumor margins.

3D Mapping of 2-HG onto MRI Tumor Reconstructions:

MRI information is critical for planning neurosurgical procedures. During the surgery, neuronavigation systems allow the neurosurgeon to register the position of surgical instruments with pre-operative plans (i.e. confirming where the tools are relative to the imaging findings). Surgeons can therefore digitally mark the site of a biopsy relative to the tumor in the MRI. Two IDH1 mutated gliomas were resected in this manner, using three-dimensional (3D) mapping, marking the positions of multiple biopsies in each case. In both cases, the 2-HG content of each stereotactic specimen was measured and normalized to its lipid signals (see materials and methods for details). This information was then correlated with the tumor cell content of each stereotactic specimen, as determined by review of both H&E and immunostains for IDH1 R132H.

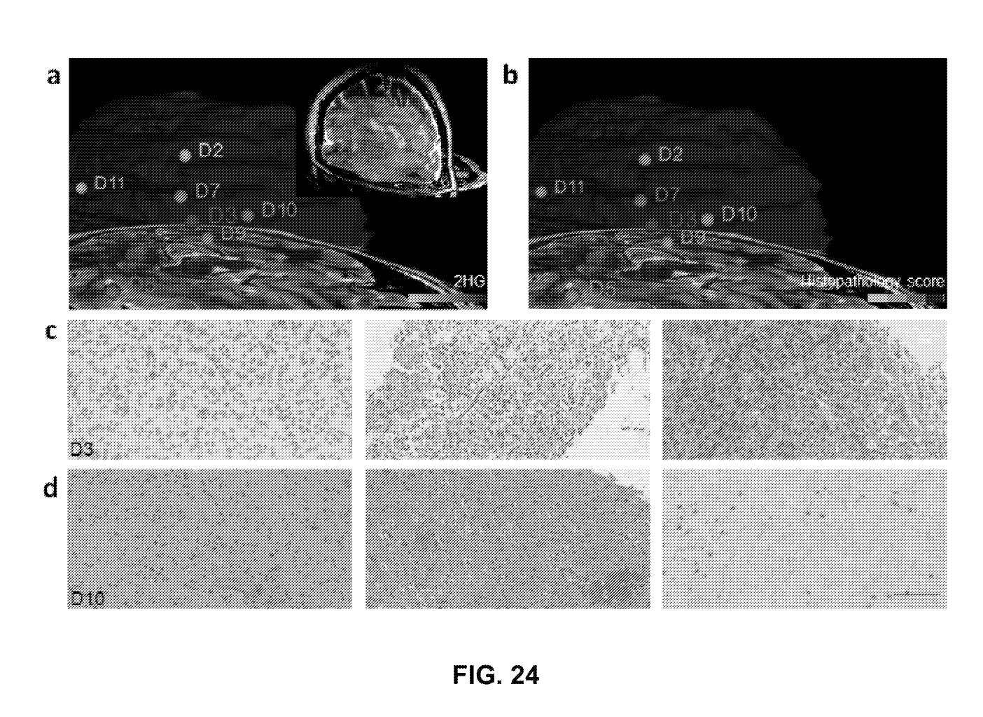

FIG. 24a shows normalized 2-HG signals that are represented with a grey scale as indicated by the scale bar; set from the lowest (lightest grey) to highest (darkest grey) levels detected from this individual case. Mass spectrometry data was acquired using a DESI LTQ instrument. Stereotactic positions were digitally registered to the pre-operative MRI using neuronavigation (BrainLab system) in a standard operating room. The inset shows the segmented tumor in light grey as it relates to brain anatomy. FIG. 24b shows histopathology scoring of tumor cell concentrations determined from reviewing of H&E stained tissue sections corresponding to samples analyzed by mass spectrometry. The scale is divided into four discrete binned grey scales corresponding (from left to right) to normal brain, low (1-29%), medium (30-59%), and high (60-100%) tumor cell concentrations. FIG. 23c shows high magnification microscopy images of H&E stained sections of sample D3 representing high tumor cell concentration. The image from the first panel is from the MS-analyzed frozen section, the middle panel is from the corresponding formalin fixed tissue section, and the last panel is from immunohistochemistry for IDH1 R132H mutant (fixed tissue). FIG. 23d shows high magnification microscopy images of H&E stained sections of sample D10 representing infiltrating tumor cells. The image from the first panel is from the MS-analyzed frozen section, the middle panel is from the corresponding formalin fixed tissue section, and the last panel is from immunohistochemistry for IDH1 R132H mutant (fixed tissue) (scale bar, 100 μm).

In the resection of an oligodendroglioma (FIG. 24), strong 2-HG signals were identified in the sample (D3) taken from the center of the tumor mass (FIG. 24a). This sample was comprised of dense tumor (FIG. 24b,c). Biopsies from the margins of the radiographic mass (e.g. D10, FIG. 24b,d) contained low concentrations of infiltrating glioma cells (FIG. 24d). In such samples, low levels of 2-HG were detected (FIG. 24a). Consistent with findings on a large panel of glioma specimens (FIG. 29 and Table 8), these stereotactic samples demonstrate that the normalized level of 2-HG correlates with the tumor cell concentration and can help define samples that are at the infiltrating border of the tumor.

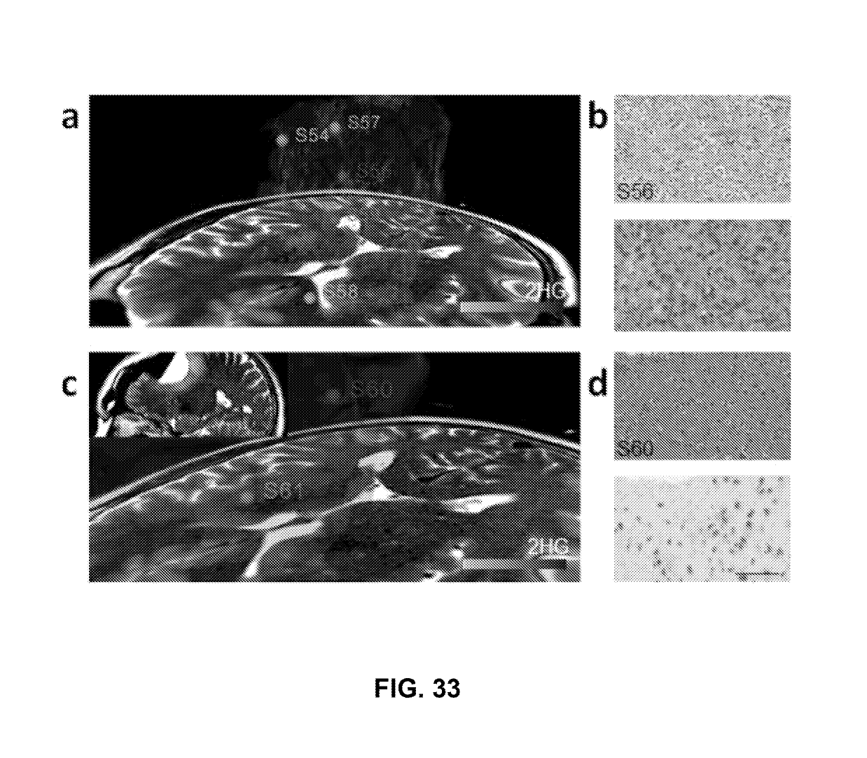

A second surgical resection was performed (FIG. 33) in the Advanced Multimodality Image Guided Operating (AMIGO) suite at Brigham and Women's Hospital that is a part of the National Center for Image-Guided Therapy. In this advanced surgical and interventional environment, MRI can be performed during the operation to see if additional tumor remains in situ. This residual tumor can then be resected before the procedure is completed.

FIG. 33a shows normalized 2-HG signal is represented with a warm grey scale as indicated by the scale bar; set from the lowest to highest levels detected from this individual case. Mass spectrometry data was acquired on a DESI Amazon Speed instrument. Stereotactic positions were digitally registered to the pre-operative MRI using neuronavigation (BrainLab system) in the AMIGO suite. FIG. 33b shows high magnification microscopy images of an H&E stained section of formalin fixed paraffin embedded tissue from sample S56 showing high tumor cell concentration (upper panel), and of immunohistochemistry for IDH1 R132H mutant (lower panel). FIG. 33c shows 2-HG over tumor volume reconstruction from the T2-weighted intraoperative MRI. The inset shows the residual lesion. FIG. 33d shows high magnification microscopy images of H&E stained sections of formalin fixed paraffin embedded tissue from sample S60 showing the presence of residual tumor cells (upper panel), and of immunohistochemistry for IDH1 R132H mutant (lower panel) (scale bar, 100 μm).

An oligoastrocytoma was resected in this second case. The location of multiple biopsy pieces were digitally registered to the pre-operative MRI and 2-HG levels were measured in each of them (FIG. 33a). The highest levels of 2-HG were detected in specimens that were taken from the center of the tumor mass and that proved to be densely cellular tumor (FIG. 33b). An intraoperative MRI of the patient's brain was taken once it appeared that the entire tumor had been removed (i.e. following an apparent gross total resection). The T2-weighted intraoperative image revealed a region that was of concern for residual tumor and surgery for more complete resection was continued based on the MRI finding (FIG. 33c). Because the areas that were concerning for residual tumor were close (just anterior) to the premotor cortex, they were carefully sampled to preserve the patient's motor function. Two additional specimens were digitally registered to the intraoperative MR image, samples S60 and S61. An equivocal 2-HG signal was detected from one sample (S61) but robust 2-HG signal was detected from the other (S60) (FIG. 33c). Microscopic review of the H&E and IDH1 R132H immunostained sections revealed only scattered tumor cells in sample S61 (<5% tumor nuclei by H&E frozen section analysis), but numerous tumor cells in sample S60 (approximately 20% tumor by H&E frozen section analysis) (FIG. 33d). This clinical example demonstrates a scenario where surveying the resection cavity with DESI MS could eventually identify areas of residual tumor without interrupting surgery for intraoperative MRI.

Real-Time Intraoperative Detection of 2-HG:

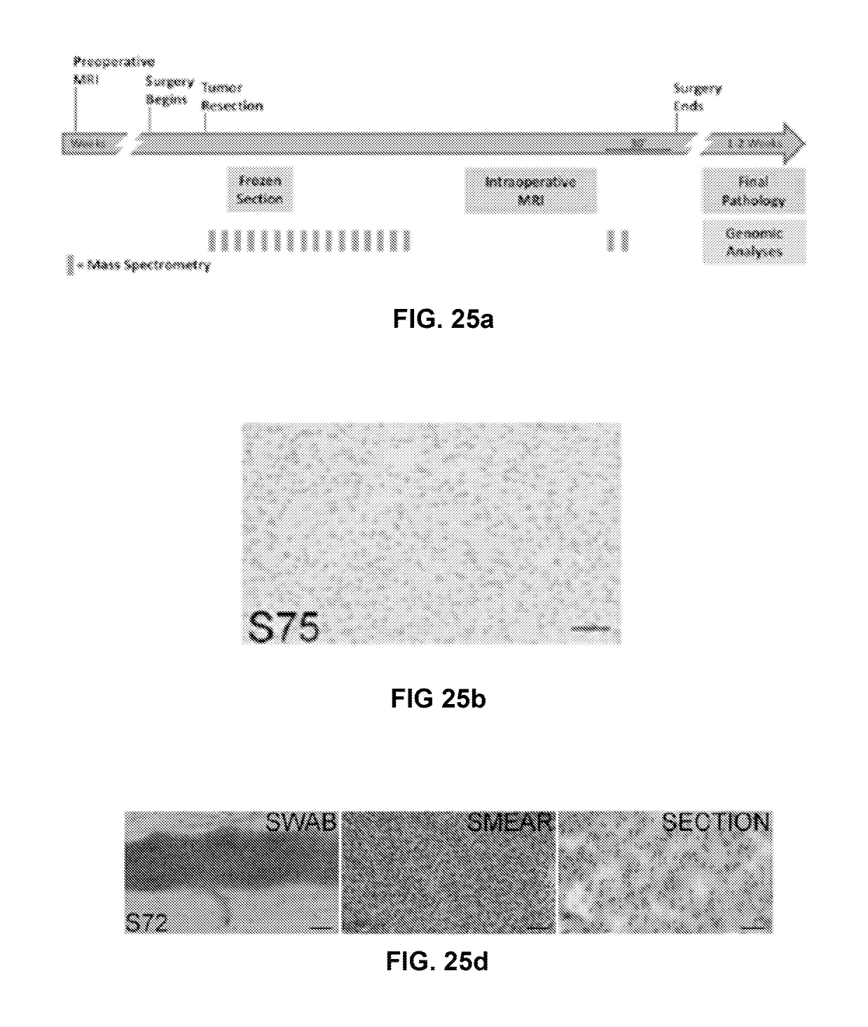

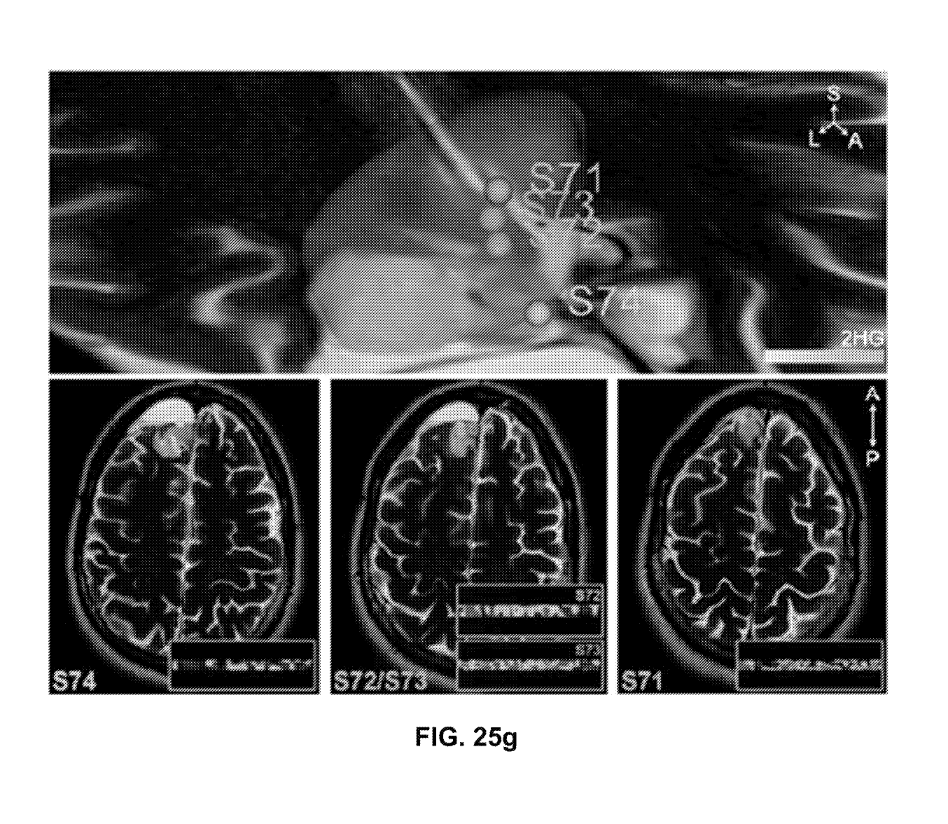

Successfully implementing DESI MS in the operative setting requires that we demonstrate the feasibility of immediately detecting 2-HG in the operating room from tissue biopsies. In FIG. 25a we outline the standard work flow for brain surgery in the AMIGO suite using current methodologies and the increased sampling that could possible with DESI MS. Time course and work flow of patient care associated with a typical 5-hour neurosurgery in the AMIGO, MRI-equipped, operative suite at Brigham and Women's Hospital. Intraoperative mass spectrometry allows for significant advances in the frequency of intraoperative tissue sampling as well as improvements in time from tissue sampling to availability of tissue analysis that can influence intraoperative surgical decision making. The schematic shows standard-of-care practices including pre- and post-operative tests (including pre-operative planning MRI, permanent surgical tumor pathology analysis, and genomic analysis of intraoperative tumor tissue samples). Also demonstrated is the intra-operative (ie surgical) workflow, including intraoperative MRI, frozen sectioning and mass spectrometry tissue analysis. All intraoperative time periods are drawn to scale according to the time required for each test. Currently, on a research basis, intraoperative mass spectrometry analysis is typically completed within 2 minutes, while frozen section analysis is completed in 20-30 minutes and intraoperative MRI requires at least 60 minutes. The time course of each intraoperative analytical measurement is measured from the time that the tissue sample is taken from the brain of the patient (or the time that the patient is readied for MRI scanning) until information from the test can returned to the surgeon to help guide the remainder of the surgery. The mass spectrometry analysis time points denote an example of the timing and frequency of representative sampling periods during an operation. Mass spectrometry time periods (hashmarked grey rectangles) connote that mass spectrometry is not yet standard of care and is a research test. To test our ability to measure 2-HG in this setting, we installed a complete DESI MS system in the AMIGO suite and monitored 2-HG levels from multiple biopsies as they were resected from two patients.

In one case, a patient had had an oligoastrocytoma (WHO grade II) resected six years earlier. Upon recurrence of the tumor, the patient was re-operated on in our AMIGO suite. Interestingly, subsequent IDH1 molecular testing showed that the tumor lacked the R132H mutation by IHC testing (FIG. 25b) but had an R132C mutation as detected by targeted sequencing (FIG. 25c). Referring to FIG. 25c, the arrow points to the IDH1 R132G (c.394C>G) mutant allele. The assayed loci were as follows: (1) KRAS 35; (2) EGFR 2236_50del R; (3) PTEN 517; (4) TP53 733; (5) IDH1 394; (6) PIK3CA 3139; (7) NOTCH1 4724 and (8) NOTCH1 4802. This information was unknown at the time of surgery. The tumor biopsies were sampled in two ways—by applying miniscule amounts of biopsy material to a standard glass slide either with a swab (the ones used for swab cultures) or by smearing a tiny tissue fragment between two glass slides (i.e. a standard smear preparation) (FIG. 25d). Within minutes, from both preparations, a peak was clearly detected that corresponded to 2-HG (m/z 147.0) (e.g. data from sample S72 is shown in FIG. 25e). Detection of 2-HG was immediately confirmed with tandem MS (FIG. 25f). After the operation, in a lab outside of the AMIGO suite, tissue sections of the remaining portion of each biopsy were analyzed with DESI MS imaging (as we had done in the validation of our methodology that is presented above) and again the presence of 2-HG in the biopsies was confirmed (FIG. 25d-f). By plotting the relative 2-HG concentration of the digitally registered samples onto the pre-operative MRI, detection of 2-HG from samples taken from the center of the tumor (S73) as well as those taken from along the tumor edge (S71 and S74) was confirmed (FIG. 25g). Stereotactic positions were digitally registered to the pre-operative MRI using neuronavigation (BrainLab system) in a standard operating room. The 3D tumor volume is shown (upper panel). Classification results of samples S74, S72, S73 and S71 are further visualized on axial sections (lower panels).

In a second case, a patient had an anaplastic oligoastrocytoma (WHO grade III) resected three years earlier. Upon recurrence of the tumor, the patient was operated on a second time, this time in the AMIGO suite. For this case, smear preparations of the biopsies (FIG. 26a) were made and 2-HG was again clearly detected in multiple biopsies from various regions of the tumor (FIG. 26b,c-left), which was confirmed by tandem MS (FIG. 26d-left). Analysis of the final pathology samples days later showed that this sample reacted with the IDH1 R123H mutation specific antibody—a multiple steps/hours immunohistochemistry assay (FIG. 26a, right). Again, after the operation in the lab outside of the AMIGO suite, the remaining portion of each biopsy was analyzed with DESI MS imaging and presence of 2-HG in the surgical biopsies was confirmed (FIG. 26a-middle, c-right, d-right).

FIG. 26a shows high magnification light microscopy images of H&E stained smear (left) and frozen tissue section (middle) of sample S92 are shown (scale bar, 200 μm). Immunohistochemistry (right) using an IDH1 R132H point mutation specific antibody on formalin-fixed and paraffin embedded (FFPE) section from an oligoastrocytoma grade III sample (S92), (scale bar, 20 μm). FIG. 26b shows normalized 2-HG signal for samples of case 28, an oligoastrocytoma grade III represented with a grey scale as indicated by the scale bar; set from the lowest (lightest grey) to highest (darkest grey) levels detected from samples for this individual case. Stereotactic positions were digitally registered to the pre-operative MRI using neuronavigation (BrainLab system) in a standard operating room. The 3D tumor volume is shown (upper panel). Classification results of samples S98, S92 and S95 are further visualized on axial sections (lower panels). Insets show negative ion mode two dimensional DESI MS images of 2-HG peak for smears of samples S98, S92 and S95. FIG. 26c shows negative ion mode DESI mass spectra obtained using an amaZon Speed ion trap from m/z 130 to 165 (Bruker Daltonics, Billerica, Mass., USA) from a smear (left) and a section (right) for sample S92. FIG. 26d shows corresponding tandem mass spectra (MS2 and MS3) of m/z 146.7 and 128.8 (smear, left) and (section, right) detected from sample S92 present a fragmentation pattern that exactly matches that of standard 2-HG.

Discussion:

It has previously been demonstrated that many tumor types can be discriminated based on their lipid profile. Here, using gliomas with IDH1 mutations as an example, it is shown that a single metabolite—that can be and was monitored during surgery with ambient mass spectrometry (MS) techniques—can rapidly provide highly relevant information: tumor classification (i.e. 2-HG expressing CNS tumors are nearly always gliomas), genotype information (i.e. 2-HG expressing tumors carry mutations in IDH1 or IDH2), and prognostic information (i.e. 2-HG expressing tumors have a more favorable outcome)—all with excellent sensitivity and specificity.

Because 70-80% of grade II and grade III gliomas as well as the majority of secondary glioblastomas contain IDH1 or IDH2 mutations, monitoring 2-HG with intraoperative MS could conceivably become routinely used for surgeries of primary brain tumors—first to classify the tumor and then, if 2-HG is present, to guide optimal resection. Presumably, the approach described here could be applicable for the resection of all 2-HG producing tumors including chondrosarcoma and cholangiocarcinoma.

Unlike more time-consuming HPLC MS approaches that are standard for quantifying 2-HG, ambient mass spectrometry techniques enable rapid data acquisition and are therefore compatible with the rigorous time constraints of surgery. Because of this, the approach described in this work was shown to provide the intraoperative guidance needed to guide the iterative process of optimizing a resection—discriminating tumor from normal brain tissue—a distinction that is of utmost importance in neurosurgery for improving patient outcomes (increased survival and decreased morbidity). One note, the spatial resolution of DESI MS is approximately 200 μm, which is ample for evaluating surgical biopsies which are often two millimeters or more in size.

While MRI is an important intraoperative tool it does have limitations. MRI is an indirect measure of the presence of a tumor; it does not definitively reveal the type of tumor that is being operated on and can sometimes not discriminate tumor from reactive adjacent tissue; each intraoperative MRI scan requires 1 hour or longer to perform and interpret; MRI is not an iterative process (i.e. generally only one scan can be performed during a procedure); and the surgeon needs to extrapolate what is learned from the MRI to judge how much more tissue needs to be removed (without being able to ask specifically and directly whether the exact tissue area in question in the surgical field is truly tumor tissue). Importantly, performing an MRI is a major interruption to the surgical procedure because the patient's cranium needs to be temporarily closed, the patient is wrapped to prevent movement in the MRI, the operating room must be cleared of all surgical instruments, nearly all personnel must ‘scrub out’ and leave the operating room, and then a team including radiologists and the surgical team has to interpret the results. For much of this, the anesthetized patient is isolated from the clinical team within the MRI scanner. Moreover, each operating room that contains an MRI machine costs over $10 million, so these intraoperative MRIs are found in only the most advanced operating rooms in the world and thereby access to these important technologies is somewhat restricted for many surgeons and patients alike. It is clear how characterizing 2-HG producing tumor tissue with DESI MS could play an important role in neurosurgery.

Other metabolites such as succinate and fumarate, which accumulate in specific tumor types, may similarly prove to be valuable metabolite markers for guiding surgery with MS approaches. As metabolomic discovery efforts intensify, the cadre of useful metabolite markers will expand significantly. This will undoubtedly increase the breadth of applications and the diagnostic utility of MS-based approaches which could utilize DESI technologies or other ambient ionization methods. Fluidly assessing molecular information, in a rapid timeframe, should allow more accurate determination of tumor margins with molecular cues (i.e. “molecular margins”), enhancing the likelihood of achieving optimal tumor resection. The low tissue requirements for our methods also raise the possibility of detection in fine-needle aspirations, core-needle biopsies, or bone marrow biopsies of a wide range of tumors types in both surgical and non-surgical settings, and some preliminary data supporting this claim are available.

Beyond the pragmatic advantages that is described, DESI MS is promising as a research tool. Two-dimensional DESI MS analysis provides adequate spatial resolution without damaging the tissue, which can subsequently be stained with H&E and visualized by standard light microscopy. Because the analyzed tissue remains intact, correlating the amount of metabolite with its originating source (i.e. stroma, blood vessel, tumor or normal non-neoplastic tissue) is possible and practical. By permitting the integration of molecular and histologic information, DESI MS can now allow us to address previously enigmatic research questions, thereby validating concepts about tumor growth and heterogeneity that are difficult to address with standard tools.

Three-dimensional tumor mapping studies hold similar promise. The information derived with DESI MS, MRI and histology, can be integrated, compared and cross-validated. This rigorous approach will help us better understand the clinical and research tools that we use as well as to shed light on tumor growth patterns and pathobiology in situ, directly in the human brain. To date, surgery remains the first and most important treatment modality for patients suffering from brain tumors. Because of the potential that is described here, metabolite-imaging mass spectrometry is a new tool with broad and powerful clinical and research applications that could transform the surgical care of patients with brain and other solid tumors.

Materials and Methods:

Tissue Samples

The tissue samples used in this study were obtained from the BWH/DFCI Neurooncology Program Biorepository collection as previously described or from stereotactic surgical cases as described in FIGS. 25 and 26. All samples were obtained and analyzed under Institutional Review Board protocols approved at BWH and DFCI. Informed written consent was obtained by neurosurgeons at BWH. The samples were sectioned for DESI MS analysis as previously described. Tumors were re-reviewed and classified in accordance with the WHO classification system by board-certified neuropathologists (SS, KLL). Resections of brain tumor lesions were performed using neuronavigation, with stereotactic mapping and spatial registering of biopsies performed as previously described. 3D-reconstruction of the tumor from MRI imaging data was achieved with 3-dimensional Slicer software package.

GBM xenografts BT116 and BT329 were derived from surgical resection material acquired from patients undergoing neurosurgery at the Brigham and Women's Hospital on an Institutional Review Board approved protocol. Briefly, tumor resection samples were enzymatically and mechanically dissociated using the MACS Brain Tumor Dissociation Kit (Miltenyi Biotech, Cambridge, Mass.) to generate single cell suspensions. Intracranial xenografts were generated by injecting 100,000 cells in the right striatum of SCID mice (IcrTac:ICRPrkdcscid; Charles River Labs, Wilmington, Mass.) and aged under standard conditions until onset of neurological symptoms. Euthanized xenografts were perfused by intra-cardiac injection of 4% paraformaldehyde and processed by standard methods for paraffin embedding.

Histopathology and Immunohistochemistry

In addition to banked snap frozen samples, all cases had tissue samples that were formalin-fixed and paraffin embedded (FFPE). Sections of FFPE tissue were stained with an anti-isocitrate dehydrogenase 1 (IDH1)-R132H antibody (clone HMab-1 from EMD Millipore) as previously described. Tissues were sectioned and immunostained as previously described. Hematoxylin and eosin (H&E) stained serial tissue sections were scanned using Mirax Micro 4SL telepathology system from Zeiss to generate digital optical images. Tumor content was evaluated by board-certified neuropathologists (S. Santagata and K. L. Ligon) through examination of H&E stained tissue sections and IDH1 R132H stained sections.

Identification of 2-Hydroxyglutarate by DESI MS

The IDH1 status of each specimen was initially evaluated by IHC of a piece of FFPE tissue. For stereotactic cases, all biopsies were less than 0.4 cm and these specimens were divided into two (one portion was frozen for DESI MS studies and the other was processed for FFPE; the latter was used for IDH1 IHC).

To determine if 2-HG could be detected directly from glioma tissue sections by DESI MS, human glioma samples were analyzed by DESI MS in the negative ion mode using either an LTQ Ion Trap (Thermo Fisher Scientific, San Jose, Calif., USA) or an amaZon speed ion trap (Bruker Daltonics, Billerica, Mass.). The solvent used in these experiments consisted of either MeOH:H2O (1:1) or ACN:DMF (1:1) with a mass from m/z 100-1100. All experiments involving the amaZon speed ion trap were carried out using a 5 kV spray voltage, 130 psi nebulizing gas (N2) and a flow rate of 0.7 μL/min.

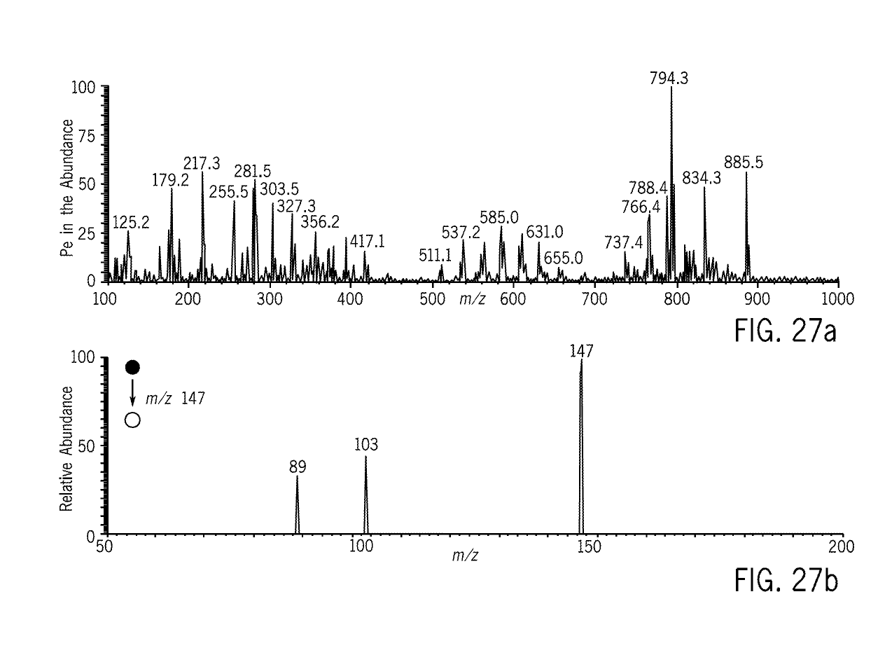

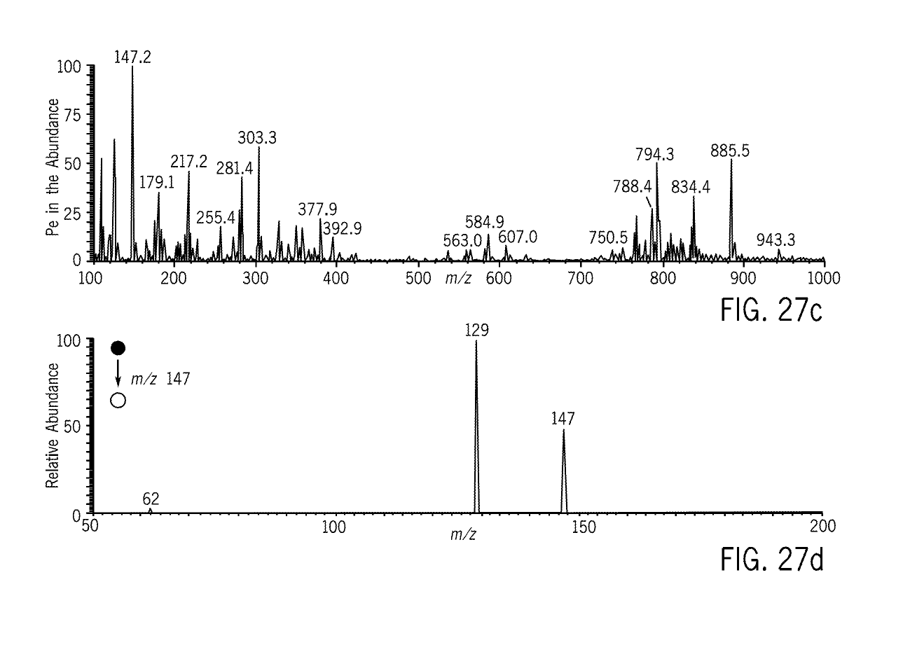

A description of the samples used in this initial testing stage of this study (analyzed with the LTQ Ion Trap) is shown in Table 8. Negative ion mode DESI MS mass spectra of samples G23, and G31 are shown in FIG. 27, using MeOH:H2O (1:1) as the solvent system. FIG. 27a shows the negative ion mode DESI mass spectrum for sample G31, a glioblastoma with wild-type IDH1. FIG. 27b shows that the tandem mass spectrum of a low abundance ion detected at m/z 147 from sample G31 presents a fragmentation pattern that does not match that of standard 2-HG. FIG. 27c shows the negative ion mode DESI mass spectrum from m/z 100 to 1000 of sample G23, an oligodendroglioma with the IDH1 R132H mutant shows high abundance of an ion at m/z 147.2. (d) Tandem mass spectrum of m/z 147.2 detected from sample G23 presents a fragmentation pattern that exactly matches that of standard 2-HG. Tandem MS analysis was used for identification of the molecular species at m/z 147.2. Further characterization was performed by MS³. The standard compound, L-α-Hydroxyglutaric acid disodium salt was purchased from Sigma-Aldrich Inc., Milwaukee, Wis. and was subjected to tandem MS experiments under the same conditions. Confirmation experiments were performed using a high-resolution LTQ Orbitrap mass spectrometer (Thermo Fisher Scientific, San Jose, Calif., USA). Further analysis was then conducted with the amaZon speed ion trap. For this instrument, the 2-HG signal was located at m/z 146.9 and was assigned using a mouse brain that contained a large tumor with the 2-HG mutation. Tandem MS and MS³ experiments were conducted on this peak to confirm its identity and found to be identical to the fragmentation pattern obtained with the LTQ instrument. Imaging of another mouse brain that had another tumor without the 2-HG mutation did not show this peak.

In total, thirty-five human gliomas samples presented in Table 8 were analyzed including oligodendrogliomas, astrocytomas, and oligoastrocytomas of different grades and varying tumor cell concentrations using both ion trap mass spectrometers. Note that as tissue analysis by DESI MS is performed without sample preparation but directly on tissue section, standard quantification of 2-HG as commonly performed with time consuming HPLC-MS protocols is not possible. One means by which relative levels of a certain molecule can be calculated is by normalizing its signal to a reference signal or set of signals obtained from the sample. In this study, the total abundance of 2-HG signal at m/z 147 was normalized to the sum of total abundances of the most abundant lipid species detected from the glioma samples by DESI MS. The mass spectra were exported as nominal mass from Xcalibur software (Thermo Fisher Scientific, San Jose, Calif., USA), and the absolute intensities of the forty most abundant lipid species within m/z 700 to 1000, which had been previously identified by tandem MS, were summed. Noise or background peaks within that m/z range were not considered. Normalization was then accomplished by dividing the total intensity of 147 by the summed intensities of the lipid species. Note that as a small contribution of background signal at the same m/z 147 was present in DESI mass spectra, MS² was performed for all samples in order to confirm the presence of 2-HG. This was especially important in some IDH1 mutant samples with low tumor cell concentrations and therefore much lower abundances of 2-HG in DESI mass spectrum. If the MS² fragmentation pattern matched that of authentic 2-HG, the sample was determined to be IDH1 mutated. Discrepancies in the fragmentation pattern or absence of detectable levels of m/z 147 were interpreted as IDH wild-type by MS analysis. Results for DESI MS analysis were obtained using two solvent systems. Note that while the solvent system DMF:ACN (1:1) favored relative abundances of low m/z ions when compared to MeOH:H2O, similar trends in 2-HG were observed for both solvents. Interestingly, the ratio of m/z 147 to the sum of lipid species correlated with the tumor cell concentration determined for the sample by histopathological evaluation of serial tissue section, providing a direct measure of the 2-HG levels in tissue. Most samples that were negative for IDH1 mutation as determined by IHC did not present 2-HG in the DESI MS mass spectra, even if the sample presented high tumor cell concentration, as confirmed by tandem MS analysis (with the exception of two samples as noted in the text).

One of the challenges in the analysis was to determine IDH1 status by DESI MS detection of 2-HG in samples with low tumor cell concentration from full mass spectral data. For these samples, low detectable values of m/z 147 could be initially assumed as an indication of IDH negative mutation. Nevertheless, MS² and MS³ of m/z 147 enabled IDH mutation status confirmation for these samples, despite the low tumor cell concentration (as low as approximately 5% of tumor). DESI MS imaging was performed for a few of the samples analyzed to evaluate the distribution of 2-HG and other diagnostic lipid species compared to tumor cell distribution in tissue.

Genetic Analysis

Archival surgical specimens were reviewed by a pathologist (S. Santagata) to select the most appropriate tumor-enriched area for analysis. Total nucleic acid was extracted from FFPE tumor tissue obtained by manual macro-dissection, followed by extraction using a modified FormaPure System (Agencourt Bioscience Corporation, Beverly, Mass.). SNaPshot mutational analysis of a panel of cancer genes that included IDH1 and IDH2, was performed as previously described.

The primers listed below were used for targeted mutation analysis at codon R132 in IDH1 (nucleotide positions c.394 and c.395) and at codons R140 and R172 in IDH2 (nucleotide positions c.418, c.419, c.514 and c.515). PCR primers: IDH1 exon 4,5′-ACGTTGGATGGGCTTGTGAGTGGATGGGTA-3′ (forward) and 5′-ACGTTGGATGGCAAAATCACATTATTGCCAAC-3′ (reverse), IDH2 exon 4a (to probe codon R140), 5′-ACGTTGGATGGCTGCAGTGGGACCACTATT-3′ (forward), and 5′-ACGTTGGATGTGGGATGTTTTTGCAGATGA-3′ (reverse), and IDH2 exon 4b (to probe codon R172), 5′-ACGTTGGATGAACATCCCACGCCTAGTCC-3′ (forward), and 5′-ACGTTGGATGCAGTGGATCCCCTCTCCAC-3′ (reverse).

Extension primers: IDH1.394 extR 5′-GACTGACTGGACTGACTGACTGACTGACTGGACTGACTGACTGAGATCCCCATAAGC ATG AC-3′, IDH1.395 extR 5′-TGATCCCCATAAGCATGA-3′, IDH2.418 extR 5′-GACTGACTGACTGACTGACTGACTGACTGACTGACTGGACTGACTGACTGACTGCCC CCA GGATGTTCC-3′, IDH2.419 extF 5′-GACTGACTGGACTGACTGACTGACTGAGTCCCAATGGAACTATCC-3′, IDH2.514 extF 5′-GACTGACTGACTGACTGACTGACTGACTGGACTGACTGACTGACTGACTGGACTGAC TGA CCCATCACCATTGGC-3′ and IDH2.515 extR 5′-GACTGACTGACTGACTGACTGACTGACTGACTGACTGGACTGACTGACTGACTGACT GGA CTGACTGAGCCATGGGCGTGC-3′.

Section V

Despite significant advances in image-guided therapy, surgeons are still too often left with uncertainty when deciding to remove tissue. This binary decision between removing and leaving tissue during surgery implies that the surgeon should be able to distinguish tumor from healthy tissue. In neurosurgery, current image-guidance approaches such as magnetic resonance imaging (MRI) combined with neuro-navigation offer a map as to where the tumor should be, but the only definitive method to characterize the tissue at stake is histopathology. While extremely valuable information is derived from this gold standard approach, it is limited to very few samples during surgery and is not practically used for the delineation of tumor margins. The development and implementation of faster, comprehensive and complementary approaches for tissue characterization are required to support surgical decision-making—an incremental and iterative process with tumor removed in multiple and often minute biopsies. The development of atmospheric pressure ionization sources makes it possible to analyze tissue specimens with little to no sample preparation.

Here, the value of desorption electrospray ionization (DESI) is highlighted as one of many available approaches for the analysis of surgical tissue. Twelve surgical samples resected from a patient during surgery were analyzed and diagnosed as glioblastoma (GBM) tumor or necrotic tissue by standard histopathology, and mass spectrometry results were further correlated to histopathology for critical validation of the approach. The use of a robust statistical approach reiterated results from the qualitative detection of potential biomarkers of these tissue types. The correlation of the MS and histopathology results to magnetic resonance images brings significant insight into tumor presentation that could not only serve to guide tumor resection, but that is worthy of more detailed studies on our understanding of tumor presentation on MRI.

Introduction:

Surgery is typically the first step for the treatment of brain tumors. To minimize the removal of functional healthy tissue, brain mapping techniques are often used prior to and during surgery. During the procedure, surgeons can use intraoperative ultrasound and MRI in centers where the technology is available, but these tools still provide limited temporal resolution (MRI) and discriminative capability (ultrasound). In addition, neither ultrasound nor MRI directly sample the tumor to determine the molecular characteristics of the tissue, thereby providing only an indirect assessment of the tumor.

Over several decades, various methods have been proposed to provide tissue discrimination including infrared or Raman spectroscopy, flow-cytometry, in vivo labeling techniques coupled with spectroscopy, and scintillation counting for the characterization of tissues in an operating room. Due to issues of complexity, limited sensitivity for properly discriminating tissues, or limited compatibility with the surgical environment none of these techniques has yet gained widespread use.

A wealth of reports have been published over the past decade on the ability of mass spectometry to discern and characterize biological tissues with increasing sensitivity and specificity. It therefore becomes very natural to return mass spectrometers back into the operating room where they were routinely used in the 1980s to sample airway gases from anesthetized patients. Now, however, they would permit the precise molecular characterization of tissue and serve as an analytical tool in image-guided therapy. Different mass spectrometry (MS) platforms will likely find themsleves interfacing with surgical decision-making at various points in the clinical workflow. MS has already proven to be useful for the characterization of intact biological tissues. For over a decade, matrixassisted laser desorption/ionization (MALDI) mass spectrometers have successfully been used for the profiling of peptides and proteins from tissues and cells in the research setting and has recently been increasingly employed for the analysis of small molecules such as lipids, drugs and their metabolites. MALDI mass spectrometry imaging (MSI) analyses of tissue have become an extremely promising tool to support decision-making in histopathology evaluation of tissue. With its ability to capture essentially a complete mass range of biomolecules that include accepted biomarkers such as proteins, MALDI MSI should assist in diagnosis providing enhanced discriminating power over visual inspection of tissue. A higher level and certainty of diagnosis provided during frozen section analysis would certainly benefit surgical decision-making in better understanding the disease faced by the surgeon. Typically, one or two samples are sent for frozen section analysis during a surgical case, and MALDI MSI could find a way to fit within comparable timelines to standard analysis. For the delineation of tumor margins though, multiple minute specimens would need to be analyzed, and the analysis should result in real-time feedback. Currently, the sample preparation steps required for MALDI MSI would not be compatible with such a workflow.

With the development of ambient ionization methods such as DESI, it is possible to perform MS analysis with essentially no sample preparation, hence making such methods compatible with the time restrictions required for intraoperative tumor diagnosis and margin delineation. In DESI, a pneumatically assisted electrospray produces charged droplets that are directed to collide with the surface of a sample. As the charged droplets collide with the sample surface they create a thin liquid film into which analytes are extracted; the impact of subsequent primary droplets releases secondary microdroplets in a process termed droplet pick-up. Following this pick-up mechanism, the standard electrospray solvent evaporation processes occur, followed by the production of dry ions of analyte either by the field desorption or charge residue process.

DESI is one of multiple atmospheric pressure ionization sources. Aimed at ease of implementation and execution, these enabling technologies produce instantaneous results from solids, aerosols, vapors and liquids situated externally to the MS, in their native environment. Examples include methods in which the energetic beam is metastable gasphase atoms and reagent ions (i.e. DART, DAPCI, FAPA, LTP), energetic droplets (i.e. DESI, EASI, JeDI), and combinations of laser radiation and ESI (i.e. ELDI, MALDESI, LAESI). Ambient methods have many applications including imaging biological tissue, and thin layer chromatography plates, as well as the direct analysis of pharmaceutical tablets and inks on banknotes and many other surfaces. DESI is readily implemented on existing commercial instruments that have a direct interface with the atmosphere and on small, field portable MS systems. Since sampling occurs outside the vacuum system of the instrument, a broad range of samples and sample forms can be presented to the mass spectrometer.

Another critical feature of DESI is that it allows MSI of sections of tissue. MSI enables to record spatially-defined biochemical information in two- and three-dimensions. DESI-MSI analysis is commonly performed by rastering the sample surface with respect to the stationary continuous flux of spray-charged droplets through an array of predefined coordinates while collecting a mass spectrum at each position containing mass-to-charge (m/z) and relative abundance information. The resulting data are concatenated into an array and selected m/z values are plotted to assess spatial distribution of intensity at specific m/z values. DESI coupled with MSI is particularly valuable in the field of tissue diagnosis for comparison with standard clinical diagnosis performed on hematoxylin and eosin (H&E) stained histological tissue sections. In contrast to extractive techniques such as liquid chromatography MS, tissue sections that have been imaged with DESI-MSI are relatively well preserved and can still be stained after the MS sampling, therefore allowing MSI data to be correlated to the exact area of tissue that was analyzed.

DESI has successfully been employed for the study of small molecules including the investigation of lipid distributions in a variety of healthy and diseased animal and human tissues exemplifying the utility of the method for determining diagnostically relevant information by MS with no sample preparation. In comparison to existing MS and optical imaging modalities, the ambient ionization methods show only modest spatial resolution. Despite this limitation, these methods have considerable benefits: they facilitate measurements outside the vacuum of the instrument, require no contrast agents or chemical-tags, and do not require further sample treatment. While very high spatial resolution is desirable for research and development, for example the nanometer range resolution achieved by technologies such as secondary ion mass spectrometry, the modest spatial resolution and fast analysis time provided by ambient MS technologies is ideal for applications in the clinical setting, especially during surgery. The miniaturization of mass spectrometers could also eventually facilitate clinical implementation.

General Workflow:

Surgery remains the most important and usually the first treatment modality for devastating brain tumors such as gliomas as well as other primary and metastatic tumors. While maximal surgical excision with the goal of gross total tumor resection is desirable, in practice, delineation of resection margins is very difficult because tumors can closely resemble normal tissue and frequently infiltrate into surrounding normal brain structures. In addition, tumors often abut or directly involve critical brain regions—too large a resection margin may increase the risk for postoperative neurologic deficits. Preoperative localization by MRI of brain tumors is used to plan the surgical intervention and to minimize postoperative deficits. But the shift in the position of brain structures that occurs following a craniotomy can lead to spatial inaccuracies.

Molecular images obtained rapidly during a surgical procedure could provide surgeons with a powerful tool for performing real-time, image-guided surgery. A variety of mapping techniques (i.e. Raman imaging, Fourier transform infrared spectroscopy imaging, diffusion tensor imaging, positron emission tomographic/single-photon emission computed tomography, electrocortical stimulation and functional magnetic resonance imaging) have been developed to provide surgeons with such understanding of the relationship of the tumor to surrounding key cortical areas for neurosurgery. Intraoperative MRI (iMRI) developed at Brigham and Women's Hospital (BWH) has provided unprecedented intraoperative visualization.

Histopathological evaluation of frozen sections from tumor biopsies is currently the only method available to provide surgeons with information about tumor type and grade. While customarily used, evaluating tumors with frozen sections has a number of significant limitations that are disruptive to the surgical workflow—in particular, the analysis of each sample requires 20 minutes or more, and typically no more than a few samples are practical to analyze during any one surgical procedure. Moreover, visual review of stained tissue sections does not provide any direct molecular information about a tumor. The use of DESI MS could help with some of these problems, by allowing continuous sampling of multiple areas within the surgical field, by providing specific information about tumor type, grade and possibly prognosis rapidly (within seconds) and by offering very specific molecular information about a sample including levels of biomarkers or therapeutic compounds.

Results highlighting the use of MS as a powerful tool in characterizing tissue for surgical-decision making are described. More specifically, DESI MS was used to distinguish necrotic tumor tissue from viable GBM tumor. Correlation between histopathological staining and DESI MS was first established to distinguish viable from non-viable tumor tissue, and built a classification model representative of the histological evaluation. A robust statistical method was then used to validate the detection of potential biomarkers. Direct correlation of mass spectrometry and histopathology results offers a level of validation that cannot be bypassed for achieving the goals of introducing this promising analytical tool in the surgical decision-making workflow and of gaining widespread acceptance by medical teams. In this approach to implement mass spectrometry into the operating suite, this validation was pushed further by correlating mass spectrometry and histopathology results to pre and intra-operative MRI. In doing so, it not only ensured the validity of the information acquired from our MS experiment and its data analysis, but also enabled clinicopathologic correlations as presented below. The case presented here addresses the discrimination between necrosis and viable tumor which challenges pre-existing knowledge of the characteristics of such tissue on MRI. This work demonstrates that mass spectrometry could play a significant role in the near- and real-time diagnosis of tumors, assist in tumor delineation, and complement MRI.

Experimental Section:

Sample Collection:

Research subjects were recruited from surgical candidates at the neurosurgery clinic of the BWH, and gave written informed consent to the Partners Healthcare Institutional Review Board (IRB) protocols. Samples were obtained in cooperation with the BWH Neurooncology Program Biorepository collection, and analyzed under Institutional Review Board-approved research protocol.

Image-Guided Neurosurgery:

All surgeries were performed with auxiliary image guidance of the BrainLab Cranial 2.1 neuronavigation system (BrainLab). Preoperative MRI-imaging sequences included full T2 (1×1×2 mm, 100×100 slice matrix) and post-contrast T1 (1×1×1 mm, 256×256 slice matrix, 176 slices), processed in the BrainLab iPlanNet 3.0 software. Standard clinical protocols were observed to obtain primary diagnosis from stained frozen sections.

Stereotactic Sample Acquisition:

After clinical frozen-section diagnosis was confirmed, additional samples were acquired during the course of clinical resection. Each sample site was localized by the neurosurgeon using the neuronavigation system pointer, and the locations were transferred for offline visualization using the OpenIGTLink protocol (client: open-source 3D Slicer software on www.Slicer.org; server: BrainLab Cranial 2.1 with OpenIGTLink license option).

Hematoxylin and Eosin Staining:

The following protocol for H&E staining was performed: 1) fix in MeOH (2 minutes), 2) rinse in water (10 dips), 3) stain in Harris modified hematoxylin solution (1.5 minutes), 4) rinse in water (10 dips), 5) blue in 0.1% ammonia (a quick dip), 6) rinse in water (10 dips), 7) counterstain in Eosin Y (8 seconds), 8) rinse and dehydrate in 100% EtOH (10 dips), 9) rinse and dehydrate again in 100% EtOH (10 dips), 10) dip in xylene (6 dips), and 11) dip in xylene again (6 dips). Sections were dried at room temperature in hood and covered with histological mounting medium (Permount®, Fisher Chemicals, Fair Lawn, N.J.) and a glass cover slide.

DESI Mass Spectrometry Imaging:

DESI-MSI was performed using an amaZon Speed™ ion trap mass spectrometer (Bruker Daltonics) equipped with a commercial DESI ion source from Prosolia, Inc. DESI-MSI was performed in a line-by-line fashion with a lateral spatial resolution of 200 μm. MS instrumental parameters used were 200° C. heated capillary temperature, 5 kV spray voltage and 4 L·min-1 dry gaz. Target mass was set to m/z 600. Seventeen microscans were averaged for each pixel in the images. The spray solvent was 1:1 acetonitrile:dimethylformamide and the solvent flow rate was 3 μL·min-1.

Statistical Analysis:

Classification models for glioma subtype, grade, and tumor cell concentration of gliomas had been previously developed using Support Vector Machine analysis in Bruker ClinProTools 3.0. New SVM classification models were calculated to classify spectra for each surgical sample (glioblastoma multiforme ‘GBM’ Vs. necrosis). Principal component analysis (PCA) and probabilistic latent semantic analysis (pLSA) were also carried out using ClinProTools 3.0 software (Bruker Daltonics). PCA is a mathematical technique designed to extract, display and rank the variance within a data set. With PCA, important information that is present in the data is retained while the dimensionality of the data set is reduced. For DESI-MSI, each mass spectrum presents a series of m/z values with specific intensities. With PCA, the set of spectra were factorized such that the constituent principal component vectors are ranked in the order of variance. In MSI, the first three PCs generally differentiate the most the samples. PCA also provides loading values (comprised between −1 and 1), originating from the calculation of the PCs, that make it easy to select the contributing peaks of each PC for further analysis. pLSA has been introduced in the MS literature as a technique to divulge latent tissue-type specific molecular signatures. For each tissue, a distinct distribution can be considered and mass spectra acquired from this tissue are analyzed as a specific combination of m/z values. In contrast to PCA, pLSA allows to directly visualize the discriminating peaks for a specific tissue type within a mass spectrum.

DESI-MSI data was converted for import to ClinProTools 3.0 using in-house software. Extracted DESI mass spectra were internally recalibrated on common spectra alignment peaks within ClinProTools 3.0. An average mass spectrum created from all single spectra was used for peak selection using the ClinProTools 3.0 internal method (based on vector quantization). Individual peak intensities were standardized across the data set. For statistical analyses, mass spectra were selected from the tissue from representative areas (GBM Vs. necrosis). Extracted DESI MS spectra acquired from D43 surgical sample were imported into ClinProTools 3.0 software. Normalization, baseline subtraction, peak peaking and spectra recalibration were automatically performed using the software.

Visualization of MRI and MS Data:

MRI data obtained were plotted in 3D Slicer (www.Slicer.org) (version 4.1). The results of MS data subjected to the described classification system were overlaid as stereotactic points rendered in grey scales representing the different tissue types.

Results and Discussion:

Mass Spectrometric Evaluation of a Glioblastoma Resection:

Twelve surgical samples (D32 to D43) were taken from a brain tumor. After a full pathologic evaluation, a final report was issued that diagnosed the tumor as a glioblastoma. This report was issued nine days following the operation. Stereotactic information was registered for ten of the biopsies (D32 to D41). Frozen sections from these surgical samples were analyzed by DESI-MSI and subsequently stained with H&E. Review of the H&E stained sections by light microscopy revealed some of these surgical samples were entirely composed of viable tumor while others were entirely composed of nonviable tumor tissue (i.e. necrotic GBM tissue) (Table 1). Because GBM tumors are composed of rapidly proliferating cells, these tumors will frequently display regions of necrosis, either focally or in large regions (termed geographic necrosis).

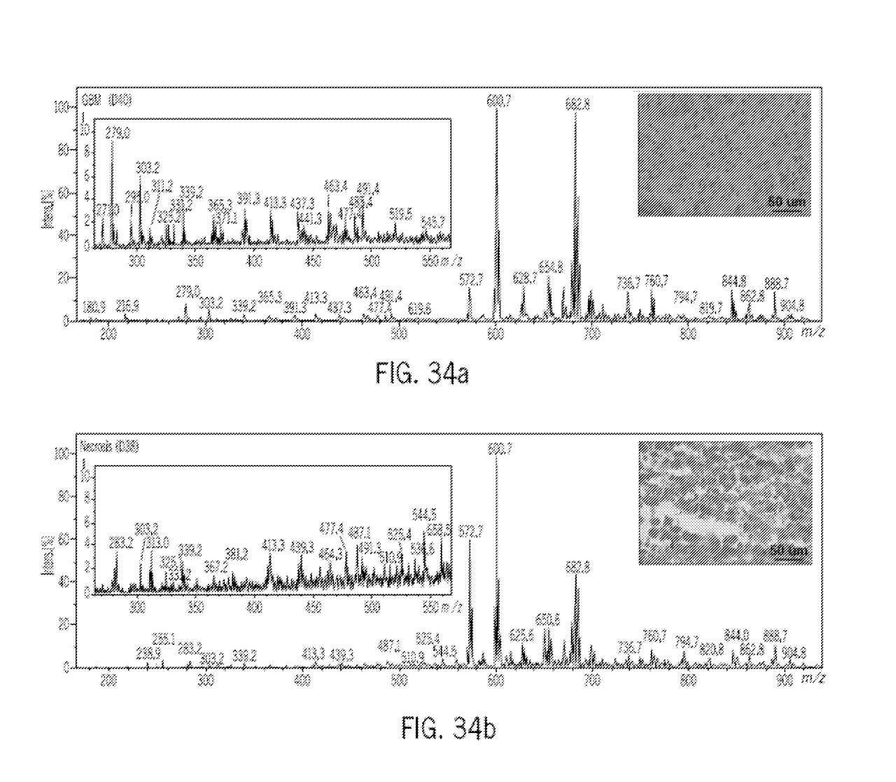

H&E stained tissue sections of surgical sample D40 showed typical histological features of GBM with a high concentration of viable tumor cells (inset of FIG. 34a) while sample D38 was entirely composed of necrotic tissue (inset of FIG. 34b). In negative-ion mode, mass spectra acquired from D40 and D38 frozen tissue sections demonstrated distinct profiles (FIG. 34) with certain ions exclusively observed in viable GBM (e.g. m/z 279.0 and m/z 391.3 from D40, FIG. 34a) and others in the necrosis region (m/z 544.5, m/z 626.6 and m/z 650.6 for D38, FIG. 34b). It was also noted that some ions were present with a higher relative abundance in one of the two surgical samples (e.g. m/z 437.3 and m/z 491.3 for D40, FIG. 34a and m/z 572.7 for D38, FIG. 34b). Corresponding ion images indicate that these ions are present throughout the tissue sections of D40 (m/z 279.0, m/z 391.3, m/z 437.3 and m/z 491.4 ions, FIG. 39a) and D38 (m/z 544.5, m/z 626.6, m/z 650.6 and m/z 572.7 ions, FIG. 39b).

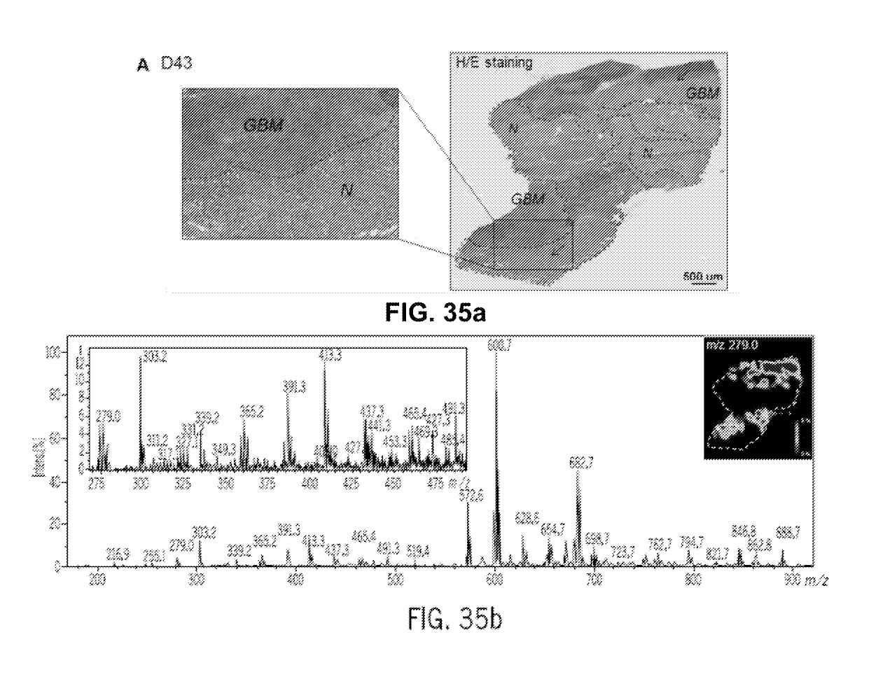

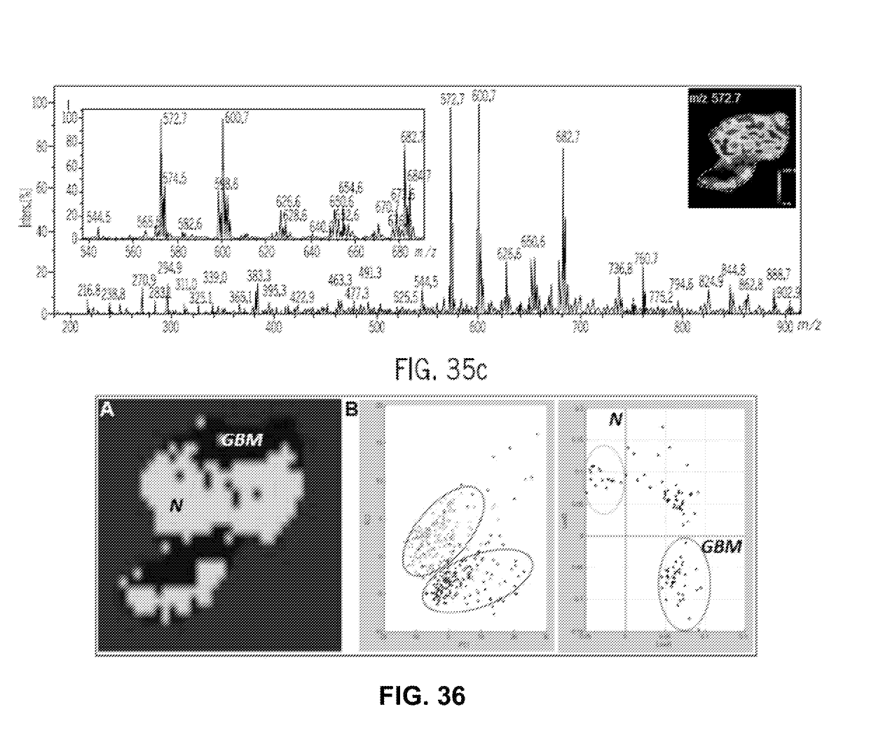

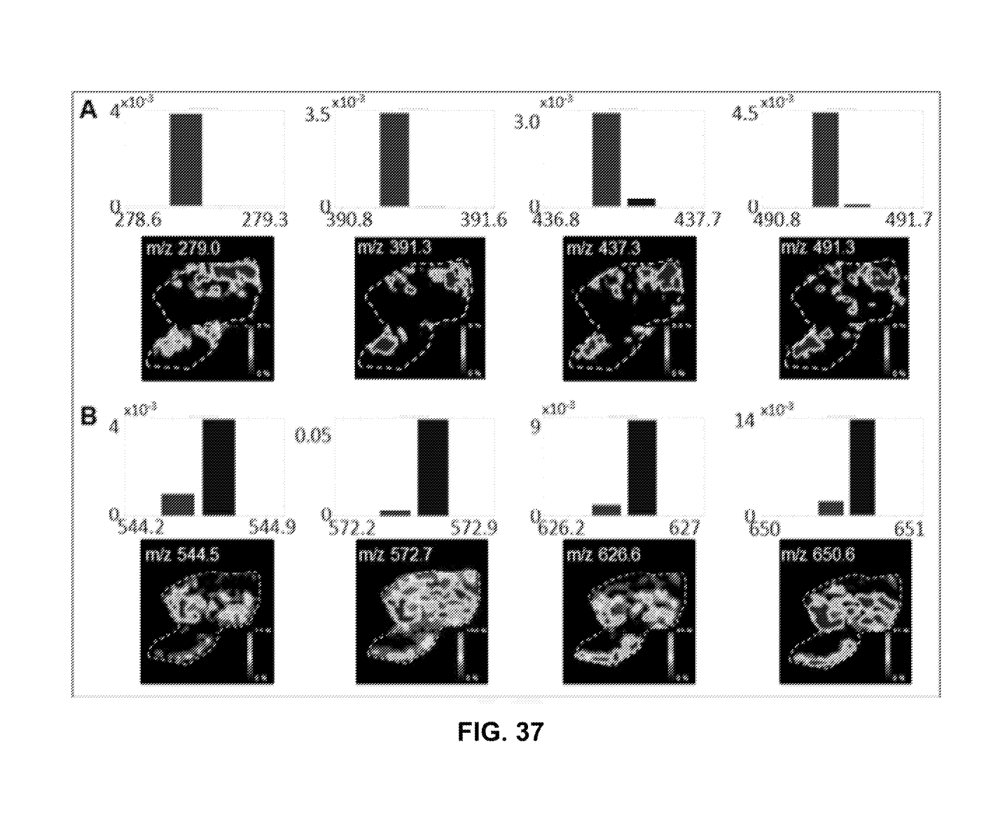

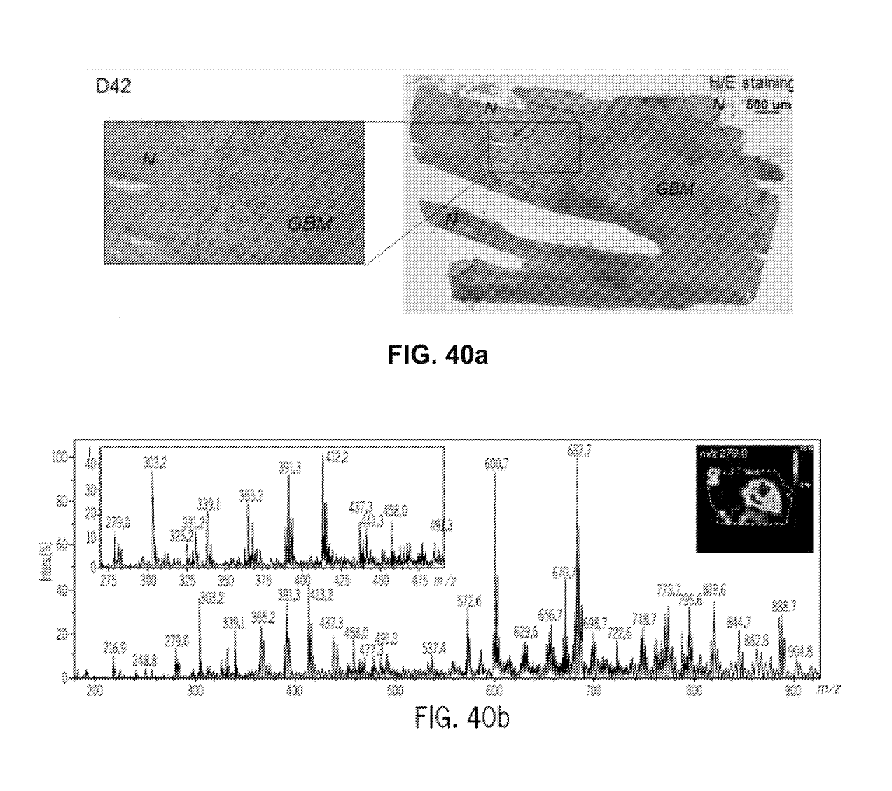

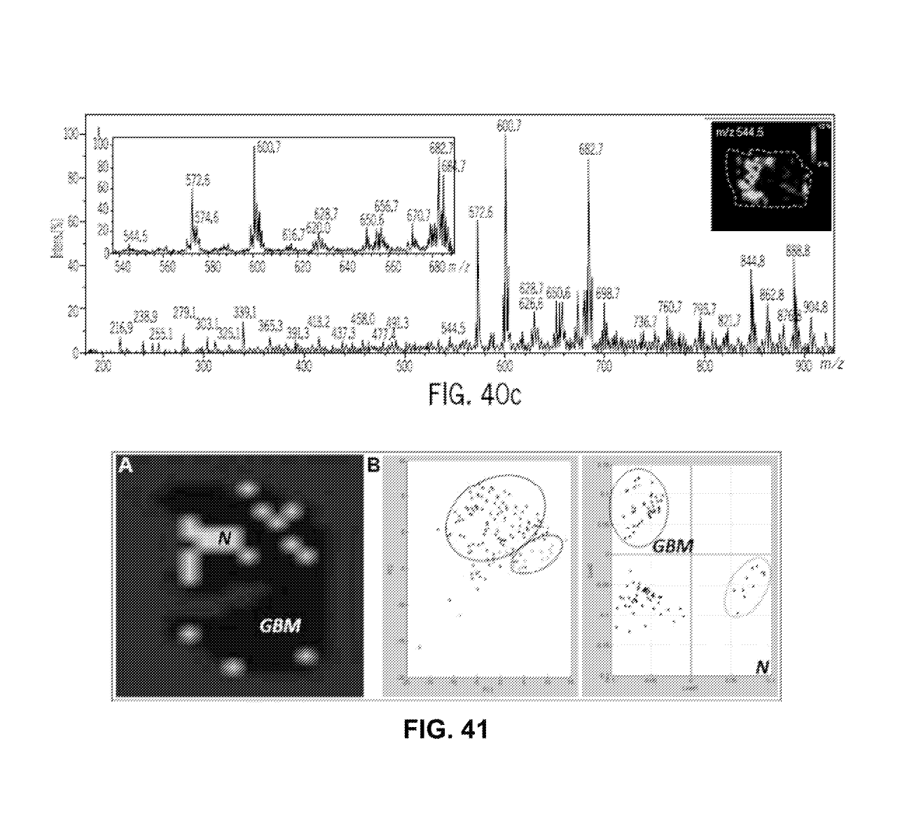

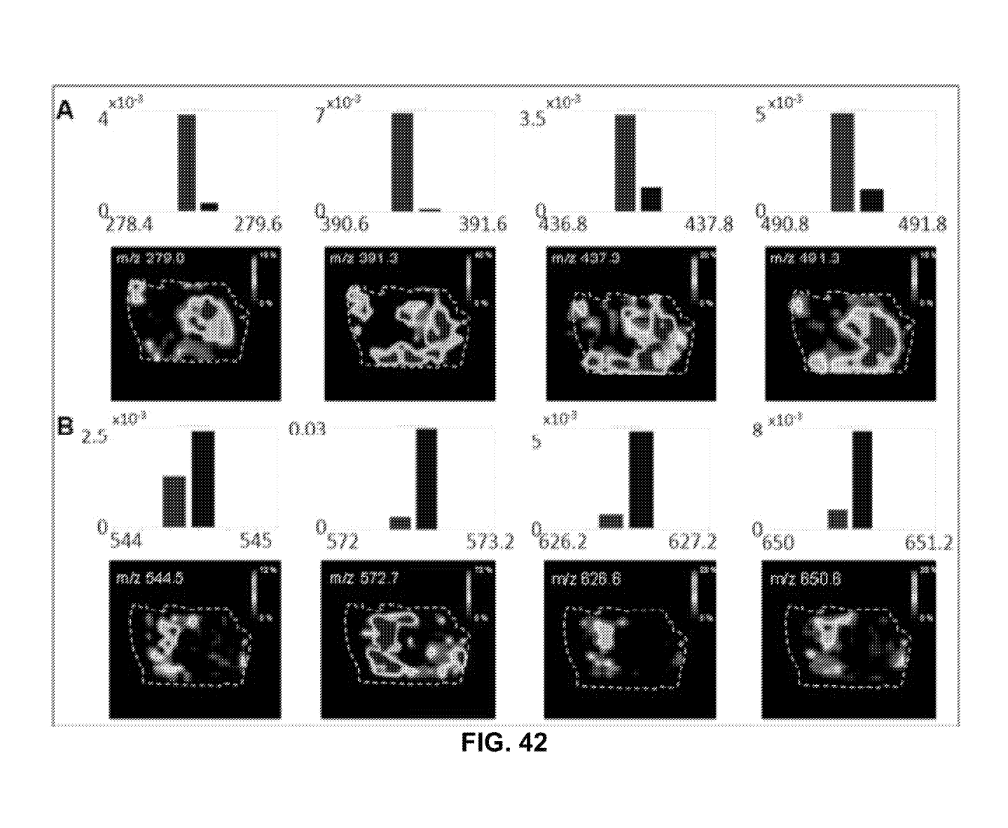

It has previously been shown that tissue specimens can be discriminated based upon the presence of specific lipid patterns. To validate the ability to distinguish viable from necrotic GBM by DESI MS molecular profiling, surgical specimens were analyzed from this GBM resection that contain within the same tissue section both viable and necrotic tumor tissue. As shown in FIGS. 35 and 40, H&E staining revealed distinct boundaries between viable GBM and necrotic tumor (N) in both surgical samples D43 (FIG. 36a) and D42 (FIG. 40a). The DESI MS data revealed that both of the lipid patterns that had been observed in sample D40 and D38 (FIG. 34) were now present in the same sample (FIGS. 35b, 35c, 40b and 40c) and were located in the appropriate histologic regions—the ion images in the insets of FIGS. 35 and 40 highlight both the areas of viable GBM (ion at m/z 279.0 FIGS. 35b and 40b) and the necrotic GBM (ions at m/z 572.7 and m/z 544.5 FIGS. 35b and 40b, respectively). Similar results were observed for other ions that we had previously identified as discriminating viable and necrotic tumor (m/z 391.3, m/z 437.3, m/z 491.3 for GBM and m/z 626.6, m/z 650.6 for necrosis; ion images of FIG. 37 for D43 and FIG. 42 for D42).

FIG. 37a shows excerpts of the m/z range showing pLSA results for peaks at m/z values 279.0, 391.3, 437.3 and 491.3. Left and right bar plots correspond to the analysis of two components, with the left bars corresponding to lipid species localized in viable GBM areas. At these m/z values, the left and right bar plots have unequal intensity for the two component spectra, indicative of a discriminatory power from the m/z values. Ion images obtained by DESI-MSI for each of these m/z values are presented below each corresponding plot. FIG. 37b shows excerpts of the m/z range of the DESI data set showing bar plots for the first two components obtained with pLSA for peaks at m/z values 544.5, 572.7, 626.6 and 650.6. The right bars here correspond to lipid species localized in areas of necrosis. Corresponding ion images to plotted m/z values are shown below each plot.

FIG. 42a shows excerpts of the m/z range showing pLSA results for peaks at m/z values 279.0, 391.3, 437.3 and 491.3. Left and right bar plots correspond to the analysis of two components, with the left bars corresponding to lipid species localized in viable GBM areas. At these m/z values, the left and right bar plots have unequal intensity for the two component spectra, indicative of a discriminatory power from the m/z values. Ion images obtained by DESI-MSI for each of these m/z values are presented below each corresponding plot. FIG. 42b shows excerpts of the m/z range of the DESI data set showing bar plots for the first two components obtained with pLSA for peaks at m/z values 544.5, 572.7, 626.6 and 650.6. The right bars here correspond to lipid species localized in areas of necrosis. Corresponding ion images to plotted m/z values are shown below each plot.

DESI-MSI for Real-Time Molecular Diagnostic:

DESI-MSI has been developed as a platform for intraoperative diagnostics. The ability to discriminate tumors of the central nervous system has been shown. This was possible not only for tumors that are highly distinct from one another (e.g. glioma from meningioma) but also for tumors that are histologically similar (e.g. discriminating low grade gliomas such as oligodendroglioma from low grade astrocytoma).

Here, it has been further demonstrated that a robust classification method can be built for discriminating viable from non-viable tumor tissue. This was readily achieved by building a classification model based on machine learning and then determining the rate of cross validation and recognition capability between GBM and necrotic tissues in other samples. The cross-validation and recognition capability demonstrated here is extremely high—in the twelve surgical samples these were 97.99% and 100%, respectively (Table 9). For D43 and D42 surgical samples, each mass spectra contributing to classify tissues as GBM or necrosis were mapped on binary images in FIGS. 36a and 41a.

PCA (FIGS. 35 and 40) and pLSA (FIGS. 36 and 41) are two statistical tools that were used in addition to the machine learning approaches to further identify discriminating peaks between tissue types.

FIG. 35a shows optical images of a D43 section H&E stained after DESI-MSI analysis. Dotted lines on the section delineate areas of necrosis “N” and viable glioblastoma “GBM” tumor. FIG. 35b shows a negative ion mode mass spectrum acquired from the viable GBM area during DESI-MSI analysis (selected mass spectrum is indicated by an arrow in FIG. 35a). In the spectrum, m/z values are detected corresponding to lipids species exclusively or preferentially detected in the GBM areas. The inset corresponds to a DESI-MSI ion image representing the repartition of an ion at m/z value 279.0. FIG. 35c shows a negative ion mode mass spectrum acquired from the necrotic area during DESI-MSI analysis (selected mass spectrum is indicated by an arrow in FIG. 35a). In the spectrum, m/z values are detected corresponding to lipids species exclusively or preferentially detected in areas of necrosis. The inset corresponds to DESI-MSI ion image representing the repartition of ion at m/z value 572.7.

FIG. 36a shows a binary image indicating spectral classification using the SVM based classifier. Mass spectra corresponding to dark grey pixels were classified as viable GBM, while light grey pixels were classified as necrosis. The left panel of FIG. 36b represents the separation of mass spectra corresponding to viable GBM (dark grey dots) and necrosis (light grey dots) according to the first two principal components (PC1, contribution of 19% and PC2, contribution of 5%). The right panel of FIG. 36b shows the loading plot generated from PCA analysis (Load 1 and Load 2). Dots correspond to m/z values. Results define three groups from these data. Each m/z value highlighted in dark grey in FIG. 36b belongs to the group circled in dark grey (GBM) whereas each m/z value highlighted in light grey in FIG. 36b belongs to the group circled in light grey (necrosis). Additional m/z values are present in these two groups and imply that additional species could be specifically detected in GBM or necrosis tissue by DESI MS.

FIG. 40a shows optical images of a D42 section H&E stained after DESI-MSI analysis. Dotted lines on the section delineate areas of necrosis “N” and viable glioblastoma “GBM” tumor. FIG. 40b shows a negative ion mode mass spectrum acquired from the viable GBM area during DESI-MSI analysis (selected mass spectrum is indicated by an arrow in FIG. 40a). In the spectrum, m/z values were detected corresponding to lipids species exclusively or preferentially detected in the GBM areas. The inset corresponds to a DESI-MSI ion image representing the repartition of an ion at m/z value 279.0. FIG. 40c shows a negative ion mode mass spectrum acquired from the necrotic area during DESI-MSI analysis (selected mass spectrum is indicated by an arrow in FIG. 40a). In the spectrum, m/z values were detected corresponding to lipids species exclusively or preferentially detected in areas of necrosis. The inset corresponds to DESI-MSI ion image representing the repartition of ion at m/z value 544.5.

FIG. 41a shows a binary image indicating spectral classification using the SVM based classifier. Mass spectra corresponding to dark grey pixels were classified as viable GBM, while light grey pixels were classified as necrosis. The left panel of FIG. 41b represents the separation of mass spectra corresponding to viable GBM (dark grey dots) and necrosis (light grey dots) according to the first two principal components (PC1, contribution of 20% and PC2, contribution of 7%). The right panel of FIG. 41b shows the loading plot generated from PCA analysis (Load 1 and Load 2). Dots correspond to m/z values. Results define three groups from these data. Each m/z value highlighted in dark grey in FIG. 41b belongs to the group circled in dark grey (GBM) whereas each m/z value highlighted in light grey in FIG. 41 belongs to the group circled in light grey (necrosis).

According to the two first principal components, PCA results show that mass spectra acquired in each region belong to the same tissue type delimited in FIG. 36a (left panel of FIG. 36b). Moreover, the loading model of the FIG. 36b (right panel) and the statistical data of Table 10 clearly indicate that m/z values presented in FIGS. 35b and 35c are specific of each tissue type. Finally, pLSA data confirm the relevance of these m/z values to discriminate the two tissue types (FIGS. 36a and 41a). Regarding the statistical study of DESI MS data of surgical case 9, it can be assumed that potential markers of GBM and necrosis could have been defined and further studies should be undertaken to specifically identify the nature of these biomolecules and assigned targeted peaks as previously described.

DESI-MSI and MRI: The Whole is Greater than the Sum of its Parts.

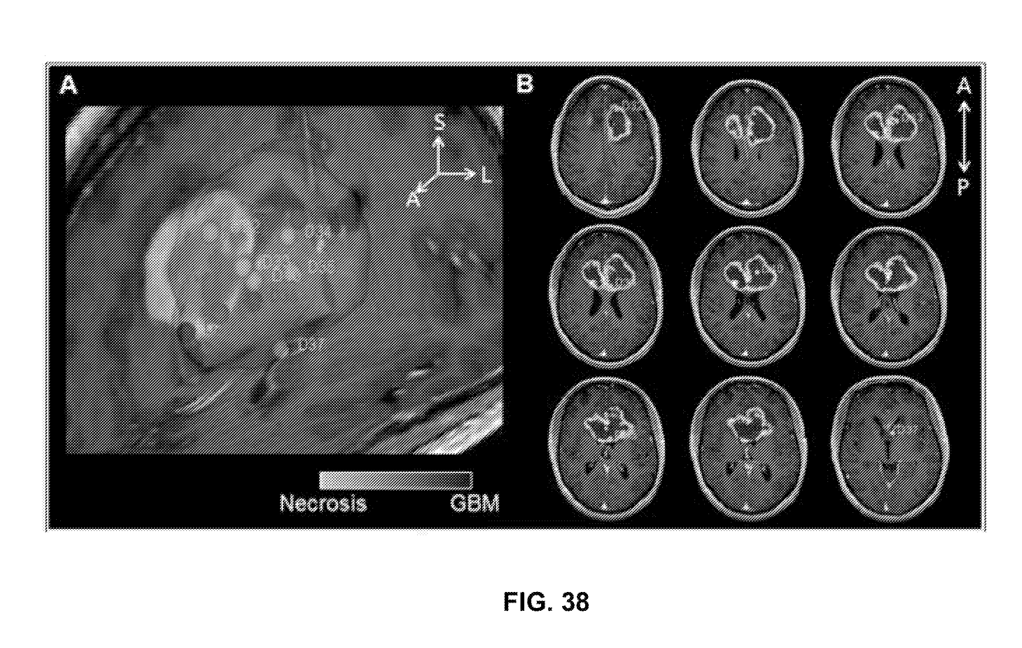

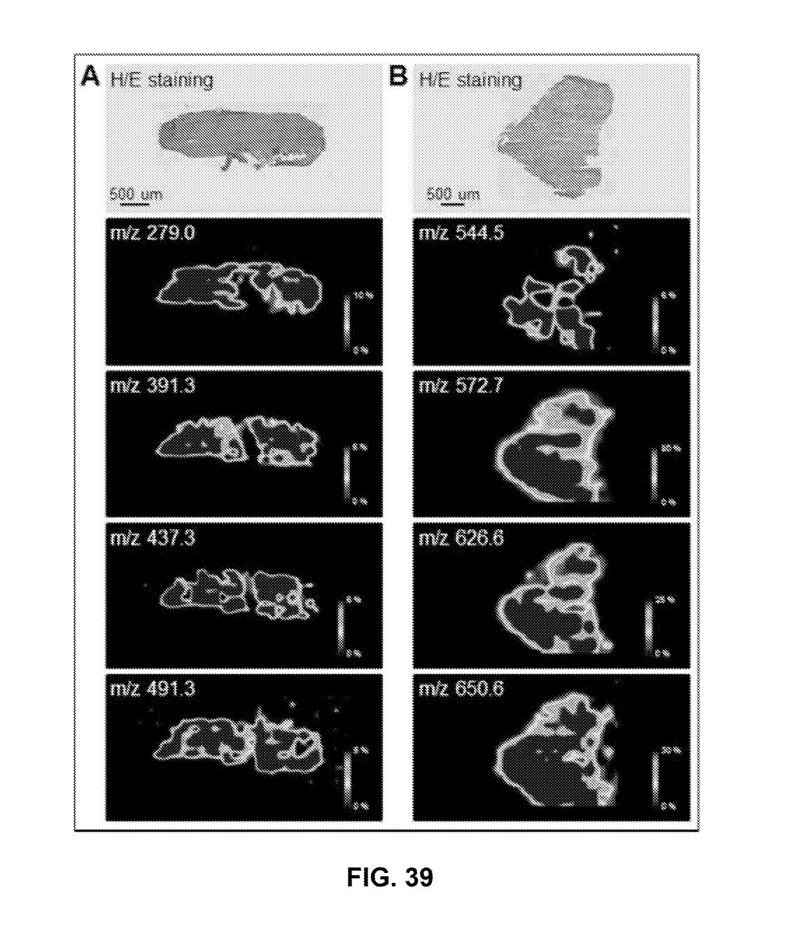

Samples from surgical case 9 were classified as GBM or necrotic tissue based on mass spectral information and the results were validated by histopathology evaluation of each specimen. Although lipid profiling provides highly specific data to discriminate tissues and define boundaries between tumor and healthy brain tissue, DESI-MSI is still an invasive technique requiring direct contact with the tissue of interest. Conversely, MRI is a non-invasive technique that may supply a mm-scale localization of the tumor, but with limited information on the tumor's chemistry. As shown in FIG. 38, 3D MR structural scans can delineate the tumor volume (FIG. 38a) and axial gadolinium-enhanced T1-weighted MR images demonstrate the spreading of this bilateral GBM across the hemisphere boundary (FIG. 38b). The majority of images in FIG. 38b show a hypodense central core, commonly associated with necrosis. This core is circled by a thick irregular ring with a shaggy inner margin typical of GBM. GBM has prominent neovascularity with abnormal blood-brain barrier, and breakdown of this barrier is thought to cause leakage of the contrast agent (i.e. gadolinium) into tissues and to be responsible for a ring-enhanced signal on enhanced T1-weighted MR images. The highest neovascularity and therefore viable tumor concentration is typically associated with the enhancing tumor ring.

Using stereotactic data about the location of the biopsies from surgical case 9, information derived from the classifiers (GBM or necrotic tissue) were mapped onto the MR images (FIG. 38). FIG. 38a shows a 3D visualization of DESI-MSI results over MRI segmented tumor volume for surgical case 9. The MRI was acquired preoperatively, and the tumor segmented and modeled using Slicer 4.0. The overall tumor volume is represented by the outlined portion. The position of each stereotactic sample was digitally registered to the pre-operative MRI using BrainLab iplan cranial 3.0, and the corresponding 3 dimensional coordinates used to render the distribution of the DESI-MSI analyses in the 3D tumor volume. The grey scale from light grey to dark grey represents the classification results from each sample between viable GBM tumor and necrosis. FIG. 38b shows classification results which are further visualized on axial sections of post-contrast T1 MR images. This view allows the correlation of viable GBM and necrosis areas, with areas of contrast enhancement. S, superior, A, anterior, L, lateral, P, posterior.

The 3D MR rendering of the segmented tumor in FIG. 38a shows the relative distribution of surgical samples as they relate to tumor presentation, while individual axial MR images more specifically correlate tissue characteristics with the uptake of contrast (FIG. 38b). As shown in FIG. 38b, DESI MS data mapping indicates that the tumor presents necrotic components both in the central and peripheral portions of the tumor. Some studies have reported that necrosis is present in 85% of cases diagnosed as GBM, but it is mainly associated with the central region of the tumor. Previous studies have also reported the propensity of radiation-induced necrosis that is the result of inflammatory cascades activated by radiation injury and exacerbated by the chronic hypoxia from endothelial remodeling. In GBM, this radiation-induced necrosis is generally observed in the periphery of the tumor, however, the patient (case 9) had not received prior radiotherapy.

Conclusion:

Surgery is the primary treatment for most brain tumors. Surgical decision-making could be improved with tools that rapidly provide molecular information about multiple biopsies or continuous sampling at the time of surgery. Ambient mass spectrometry techniques that can provide near-real time molecular information from tissue samples hold great potential in this area. With DESI MS, the ability to classify tumors, define tumor subtypes, and identify tumor grade has been shown. Here it is shown that in surgical resection specimens, necrotic tumor tissue, an indicator of a high-grade malignancy, can be readily identified and necrotic tumor tissue can be distinguished from viable tumor regions. As DESI MS is applied to a broad range of human malignancies the molecular correlates of a range of histologic features, many of which have become diagnostic hallmarks of cancer (such as necrosis in the diagnosis of GBM), will be able to be defined. Many of these insights will rely on the use of powerful machine learning and statistical tools to assist in turning the vast data sets acquired by mass spectrometry into usable tumor classifiers that are ultimately useful for real-time applications. As more and more is done, DESI MS could have a significant role for a broad range of diagnostic applications including defining the boundaries between tumor and normal tissue, diagnosing image-guided needle biopsies and determining prognostic and predictive information for guiding patient care. The siting of a mass spectrometer into the AMIGO at BWH provides with invaluable opportunities to validate mass spectrometry findings for a variety of surgical diseases tackled by the growing field of mass spectrometry imaging and to continue technology development with the hope of improving patient care.

The invention has been described in connection with what are presently considered to be the most practical and preferred embodiments. However, the present invention has been presented by way of illustration and is not intended to be limited to the disclosed embodiments. Specifically, the above specific methods used are exemplary of the inventive concept and may be altered while still falling within the scope and spirit of the invention. Accordingly, those skilled in the art will realize that the invention is intended to encompass all modifications and alternative arrangements within the spirit and scope of the invention as set forth in the appended claims.

1. A system for determining a presence of cancer in a tissue sample, the system comprising:
a sampling probe;
a mass spectrometry apparatus in communication with the sampling probe and configured to receive the tissue sample and analyze the tissue sample using a mass spectrometry process to generate mass spectrometry data;
a computer system including a computer processor having access to a non-transitory, computer-readable storage medium having stored thereon instructions that cause the computer processor to:
receive the mass spectrometry data from the mass spectrometry apparatus;
analyze the mass spectrometry data to determine a presence of at least one potential biomarker indicating the presence of cancer in the tissue sample;
access a database of at least one of biomarker information and biomarker analysis algorithms;
analyze the at least one potential biomarker using the at least one of the biomarker information and biomarker analysis algorithms to determine a presence of the at least one potential biomarker in the tissue sample; and
determine, from the presence of the at least one potential biomarker in the tissue sample, a likelihood of cancer in the tissue sample; and
a report generator configured to deliver a report indicating the likelihood of cancer in the tissue sample.

2. The system of claim 1, wherein the sampling probe includes an aspiration pathway in communication with a tip and the mass spectrometry apparatus is in communication with the sampling probe via the aspiration pathway, the tip of the sampling probe configured to vibrate in response to ultra-sonic energy to remove the tissue sample.

3. (canceled)

4. The system of claim 1, wherein the at least one potential biomarker includes a lipid.

5. The system of claim 1, wherein the at least one potential biomarker includes one of m/z 89.1, m/z 281.3, m/z 282.24, m/z 303.3, m/z 304.24, m/z 365.4, m/z 366.35, m/z 391.4, m/z 392.37, m/z 413.4, m/z 445.4, m/z 572.6, m/z 626.8, m/z 656.8, and m/z 682.8.

6. The system of claim 1, wherein the computer processor is further caused to determine a relative abundance of the at least one potential biomarker and wherein the report generator is configured to indicate a higher relative abundance of the at least one potential biomarker as compared to healthy tissue as indicating cancer in the tissue sample.

7. The system of claim 1, wherein the report includes a chart of a relative abundance of all detected ions.

8. The system of claim 1, wherein the mass spectrometry apparatus includes a desorption electrospray ionization apparatus.

9. The system of claim 1, wherein the report generator is configured to be mounted within a procedure room.

10. (canceled)

11. The system of claim 1, wherein the report generator is configured to generate a mass spectrometry report.

12-13. (canceled)

14. A method for determining a presence of cancerous cells within a subject during a surgical procedure to remove the cancerous cells, the method comprising:
harvesting the cancerous cells;
positioning a sampling probe proximate to an analysis site;
acquiring a tissue sample from the analysis site using the sampling probe; providing the tissue sample from the sampling probe to a mass spectrometry system;
conducting a mass spectrometry procedure on the tissue sample to produce a spectrographic report;
analyzing the spectrographic report to determine a presence of a biomarker indicating a presence of cancer in the tissue sample from the subject; and
generating a report indicating a likelihood of cancer existing in the analysis site.

15. The method of claim 14, wherein the biomarker includes a lipid.

16. The method of claim 14, wherein the biomarker includes one of m/z 89.1, m/z 281.3, m/z 282.24, m/z 303.3, m/z 304.24, m/z 365.4, m/z 366.35, m/z 391.4, m/z 392.37, m/z 413.4, m/z 445.4, m/z 572.6, m/z 626.8, m/z 656.8, and m/z 682.8.

17. The method of claim 14, wherein the step of analyzing includes determining a relative abundance of the biomarker and wherein the relative abundance of the biomarker is higher in a cancerous tissue sample than a healthy tissue sample.

18. The method of claim 14, wherein the report includes a chart of a relative abundance of all detected ions.

19. The method of claim 14, wherein the mass spectrometry procedure includes a desorption electrospray ionization.

20. (canceled)

21. The method of claim 14, the method further comprising performing histological staining of the tissue sample.

22-24. (canceled)

25. The method of claim 14, the method further comprising indicating a boundary between cancerous cells and non-cancerous cells using the report.

26. The method of claim 14, the method further comprising conducting an imaging procedure.

27. The method of claim 26, the method further comprising stereotactically tracking a location of a tip of the sampling probe.

28. The method of claim 27, the method further comprising correlating the report to the tracked location of the tip within an image produced by the imaging procedure.
